(19) United States
(12) Patent Application Publication
(10) Pub. No.: US 2016/0185745 A1
(43) Pub. Date: Jun. 30, 2016

(54) ANALOGS OF VINAXANTHONE AND XANTHOFULVIN, METHODS OF SYNTHESIS, AND METHODS OF TREATMENTS THEREOF

(71) Applicant: BOARD OF REGENTS, THE UNIVERSITY OF TEXAS SYSTEM, Austin, TX (US)

(72) Inventors: Dionicio SIEGEL, Austin, TX (US); Matthew CHIN, Austin, TX (US); Anders ELIASEN, Austin, TX (US); Abram AXELROD, Hurley, NY (US)

(73) Assignee: BOARD OF REGENTS, THE UNIVERSITY OF TEXAS SYSTEM, AUSTIN, TX (US)

(21) Appl. No.: 14/910,752

(22) PCT Filed: Aug. 7, 2014
(86) PCT No.: PCT/US2014/050050

§ 371 Date: Feb. 8, 2016

Related U.S. Application Data
(60) Provisional application No. 61/863,237, filed on Aug. 7, 2013.
(60) Provisional application No. 61/866,430, filed on Aug. 15, 2013.

(51) Int. Cl. C07D 311/86; A61K 31/194; A61K 31/353

(52) U.S. Cl. CPC C07D 311/86; A61K 31/353; A61K 31/194

(57) ABSTRACT

The present invention relates generally to methods of synthesis of vinaxanthone and xanthofulvin, novel analogs, and methods of use thereof.

This application claims the benefit of U.S. Provisional Application Ser. No. 61/863,237 filed Aug. 7, 2013, and U.S. Provisional Application Ser. No. 61/866,430, filed Aug. 15, 2013 the entire contents of which are hereby incorporated by reference.

This invention was made with government support under Grant No. CHE-1151708 awarded by the National Science Federation (NSF). The government has certain rights in the invention.

### BACKGROUND OF THE INVENTION

I. Field of the Invention

The present invention relates to chemistry, chemical synthesis, diabetes, and neural regeneration. The present invention relates generally to novel method of preparing vinaxathone and xanthofulvin, methods of treatment, and novel analogs, thereof.

II. Description of Related Art

The Penicillium sp. SPF-3059 derivatives, xanthofulvin and vinaxanthone, are pharmaceutical leads in axonal regeneration. Kaneko et al. (2006) illustrated the regenerative properties of xanthofulvin following complete spinal cord transection in adult rats, and Omoto et al. (2012) showed the neuroregenerative properties of vinaxanthone in corneal transplantation experiments in mice models. However, genetic knockdown of Sema3A does not garner the pronounced regenerative effects characteristic of xanthofulvin or vinaxanthone, suggesting that the mode of action for xanthofulvin and vinaxanthone is unclear. Given the therapeutic efficacy of these compounds and the potential for analogs to be even more efficacious, methods of producing these compounds and analogs are of industrial importance.

The first synthesis of vinaxanthone was performed by Tatsuta et al. through the intermolecular Diels-Alder (IMDA) reaction of two molecules of a protected vinyl ketone precursor made in 14 steps. This synthesis provided the first biomimetic pathway for vinaxanthone synthesis but produced a mix of products in the final IMDA reaction (Tatsuta, et al., 2007). Unfortunately, the synthesis does not produce a modular nature to obtain analogs of vinaxanthone and thus new methods of synthesis are needed.

### SUMMARY OF THE INVENTION

In some aspects of the present disclosure, the present disclosure provides a method of preparing a compound of the formula:

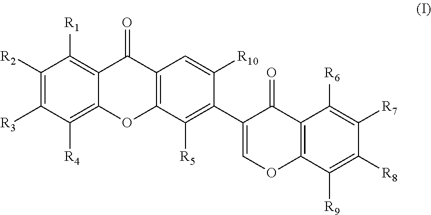

wherein: R₁, R₂, R₃, R₄, R₆, R₇, R₈, and R₉ are each independently hydrogen, amino, carboxy, halo, hydroxy, mercapto, or alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅, wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and R₅ and R₁₀ are each independently hydrogen, acyl_{(C≦12)}, or substituted acyl_{(C≦12)}; or a salt thereof; comprising reacting in a reaction mixture a compound of the formula:

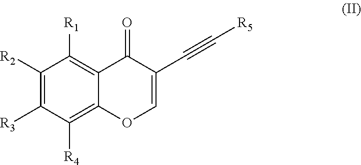

wherein: R₁, R₂, R₃, and R₄ are each independently hydrogen, amino, carboxy, halo, hydroxy, mercapto, or alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅, wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and R₅ is hydrogen, acyl_{(C≦12)}, or substituted acyl_{(C≦12)}; with water in a first solvent. In some embodiments, the compound of formula I is further defined as:

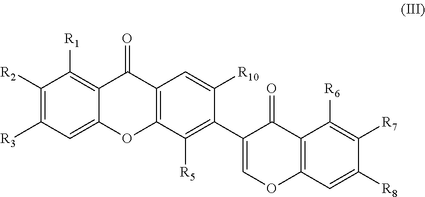

wherein: R₁, R₂, R₃, R₆, R₇, and R₈ are each independently hydrogen, amino, carboxy, halo, hydroxy, mercapto, or alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅, wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and R₅ and R₁₀ are each independently hydrogen, acyl_{(C≦12)}, or substituted acyl_{(C≦12)}; or a salt thereof. In some embodiments, the compound of formula I is further defined as:

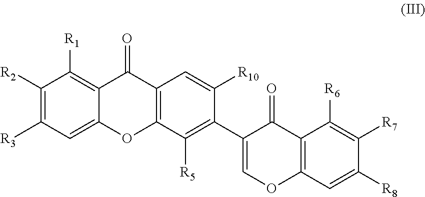

wherein: R₁, R₂, R₃, R₆, R₇, and R₈ are each independently hydrogen, carboxy, hydroxy, or alkoxy_{(C≦12)}, acyl_{(C≦12)}, substituted alkoxy_{(C≦12)}, substituted acyl_{(C≦12)}, —OX₁, or —C(O)OX₅, wherein: X₁ is a hydroxy protecting group and X₅ is a carboxy protecting group; and R₅ and R₁₀ are each independently hydrogen, acyl_{(C≦12)}, or substituted acyl_{(C≦12)}; or a salt thereof. In some embodiments, the compound of formula I is further defined as:

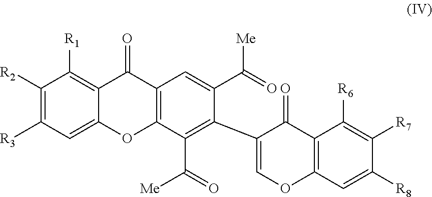

wherein: R₁, R₂, R₃, R₆, R₇, and R₈ are each independently hydrogen, carboxy, hydroxy, or alkoxy_{(C≦12)}, acyl_{(C≦12)}, substituted alkoxy_{(C≦12)}, substituted acyl_{(C≦12)}, —OX₁, or —C(O)OX₅, wherein: X₁ is a hydroxy protecting group and X₅ is a carboxy protecting group; or a salt thereof. In some embodiments, R₁ is hydrogen. In other embodiments, R₁ is carboxy. In other embodiments, R₁ is hydroxy. In some embodiments, R₁ is —OX₁. In some embodiments, X₁ is pivaloyl or methoxymethyl. In other embodiments, R₁ is —C(O)OX₅. In some embodiments, X₅ is t-butyl. In some embodiments, R₂ is hydrogen. In other embodiments, R₂ is carboxy. In other embodiments, R₂ is hydroxy. In other embodiments, R₂ is —OX₁. In some embodiments, X₁ is pivaloyl or methoxymethyl. In other embodiments, R₂ is —C(O)OX₅. In some embodiments, X₅ is t-butyl. In some embodiments, R₃ is hydrogen. In other embodiments, R₃ is carboxy. In other embodiments, R₃ is hydroxy. In other embodiments, R₃ is —OX₁. In some embodiments, X₁ is pivaloyl or methoxymethyl. In other embodiments, R₃ is —C(O)OX₅. In some embodiments, X₅ is t-butyl. In some embodiments, R₆ is hydrogen. In other embodiments, R₆ is carboxy. In other embodiments, R₆ is hydroxy. In other embodiments, R₆ is —OX₁. In some embodiments, X₁ is pivaloyl or methoxymethyl. In other embodiments, R₆ is —C(O)OX₅. In some embodiments, X₅ is t-butyl. In some embodiments, R₇ is hydrogen. In other embodiments, R₇ is carboxy. In other embodiments, R₇ is hydroxy. In other embodiments, R₇ is —OX₁. In some embodiments, X₁ is pivaloyl or methoxymethyl. In other embodiments, R₇ is —C(O)OX₅. In some embodiments, X₅ is t-butyl. In some embodiments, R₈ is hydrogen. In other embodiments, R₈ is carboxy. In other embodiments, R₈ is hydroxy. In other embodiments, R₈ is —OX₁. In some embodiments, X₁ is pivaloyl or methoxymethyl. In other embodiments, R₈ is —C(O)OX₅. In some embodiments, X₅ is methyl or t-butyl. In some embodiments, R₄ is hydrogen. In some embodiments, R₉ is hydrogen. In some embodiments, R₅ is acyl_{(C≦12)} or substituted acyl_{(C≦12)}. In some embodiments, R₅ is —C(O)Me. In some embodiments, R₁₀ is acyl_{(C≦12)} or substituted acyl_{(C≦12)}. In some embodiments, R₁₀ is —C(O)Me or —C(O)OMe. In some embodiments, R₁₁ is acyl_{(C≦12)} or substituted acyl_{(C≦12)}. In some embodiments, R₁₁ is —C(O)Me or —C(O)OMe. In some embodiments, R₁₂ is acyl_{(C≦12)} or substituted acyl_{(C≦12)}. In some embodiments, R₁₂ is —C(O)Me or —C(O)OMe. In some embodiments, the compound of formula I is further defined as:

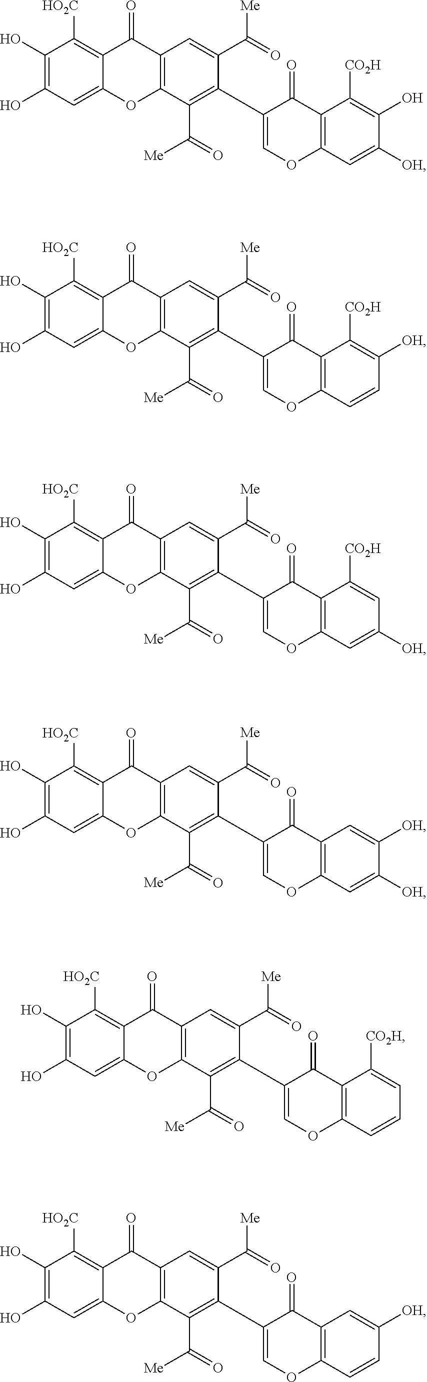 [SMILES: CC(=O)c1cc2c(=O)c3c(C(=O)O)c(O)c(O)cc3oc2c(C(C)=O)c1-c1coc2cc(O)c(O)c(C(=O)O)c2c1=O ; CC(=O)c1cc2c(=O)c3c(C(=O)O)c(O)c(O)cc3oc2c(C(C)=O)c1-c1coc2ccc(O)c(C(=O)O)c2c1=O ; CC(=O)c1cc2c(=O)c3c(C(=O)O)c(O)c(O)cc3oc2c(C(C)=O)c1-c1coc2cc(O)cc(C(=O)O)c2c1=O ; CC(=O)c1cc2c(=O)c3c(C(=O)O)c(O)c(O)cc3oc2c(C(C)=O)c1-c1coc2cc(O)c(O)cc2c1=O ; CC(=O)c1cc2c(=O)c3c(C(=O)O)c(O)c(O)cc3oc2c(C(C)=O)c1-c1coc2cccc(C(=O)O)c2c1=O ; CC(=O)c1cc2c(=O)c3c(C(=O)O)c(O)c(O)cc3oc2c(C(C)=O)c1-c1coc2ccc(O)cc2c1=O]

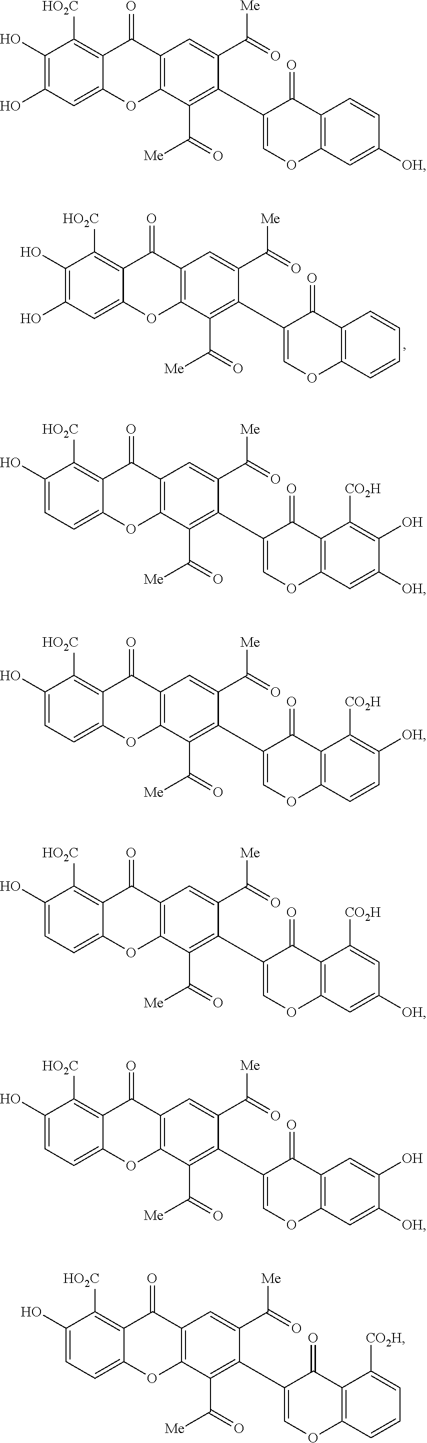

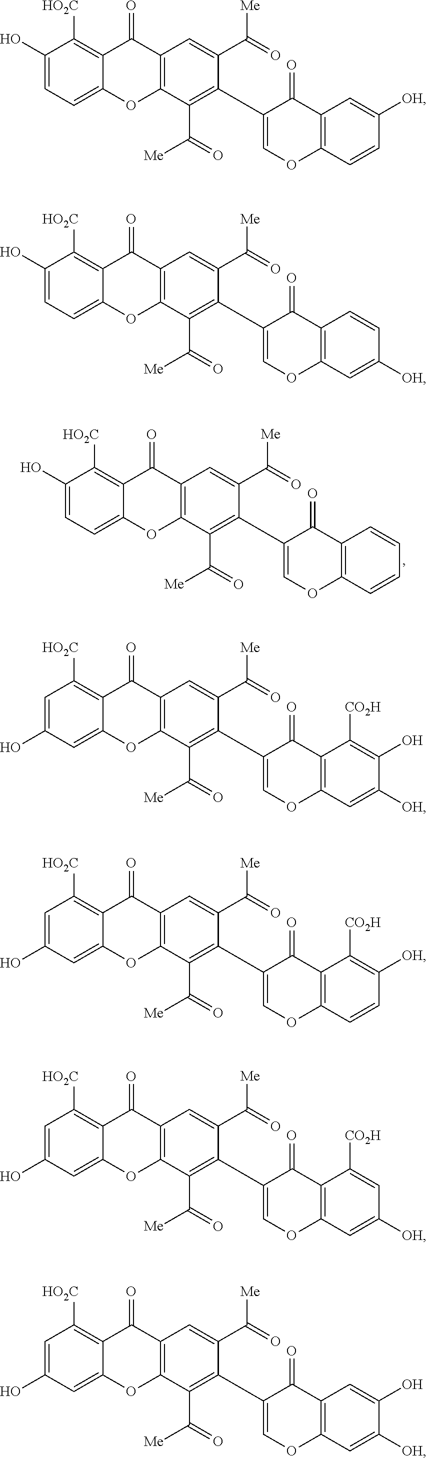

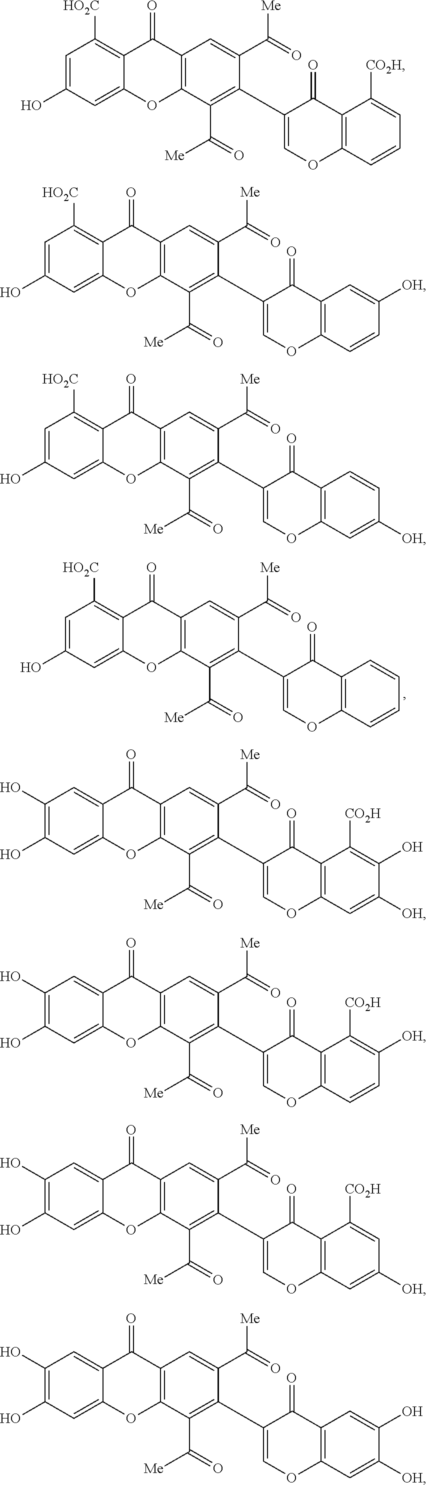

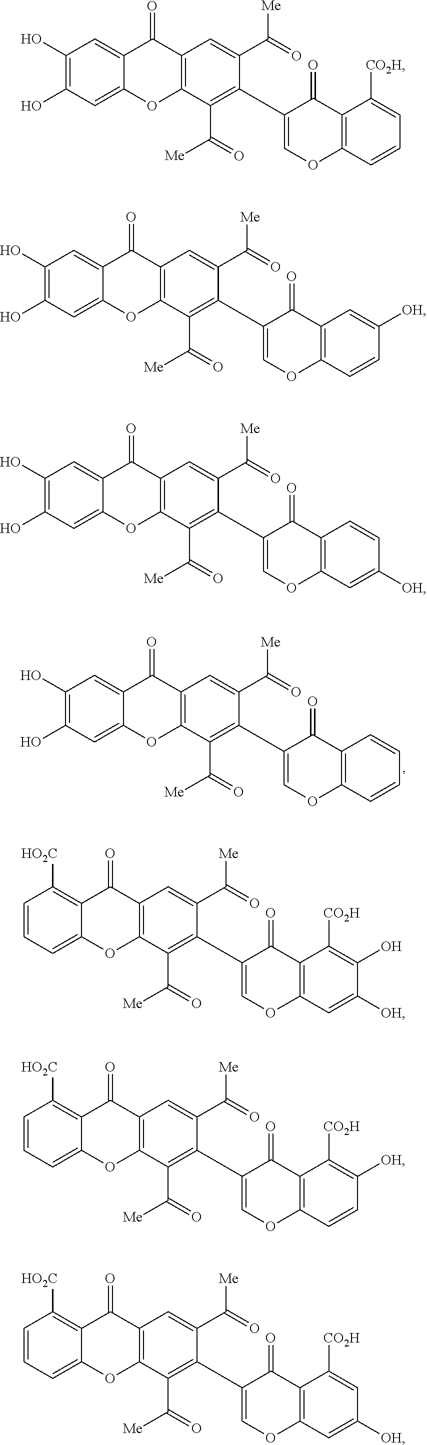

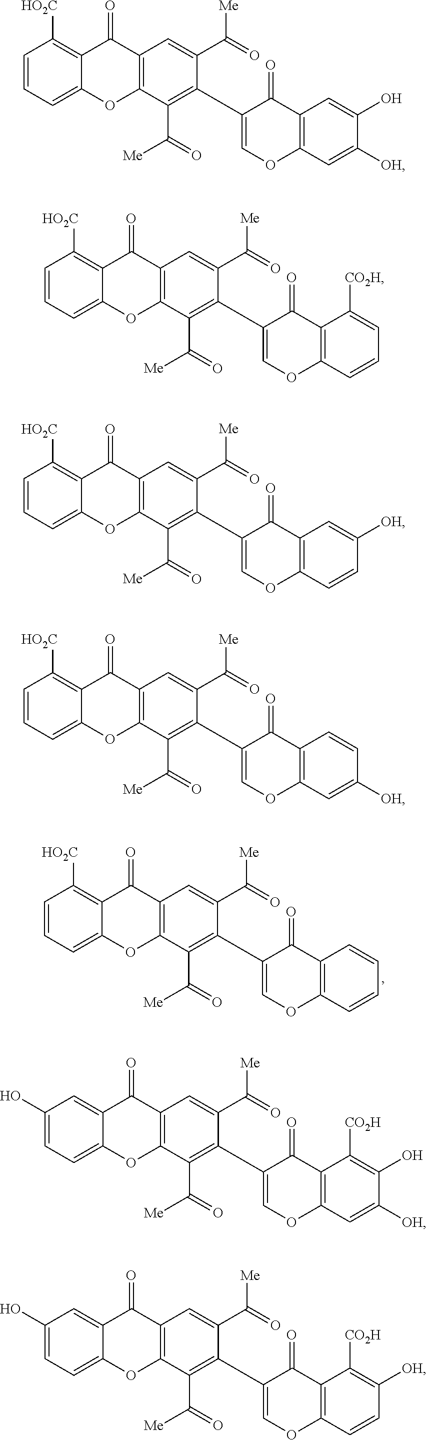

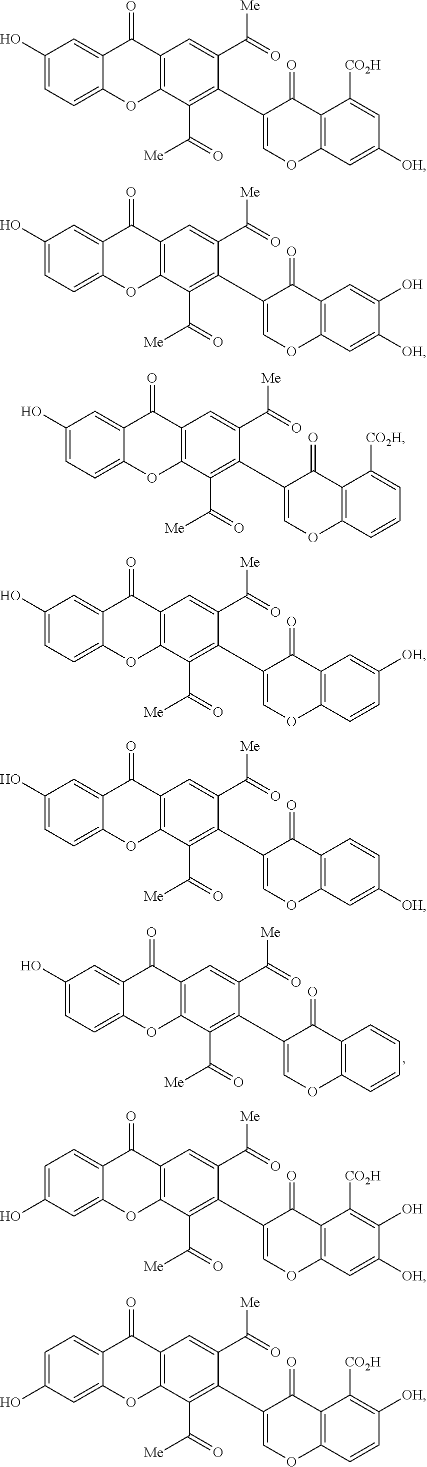

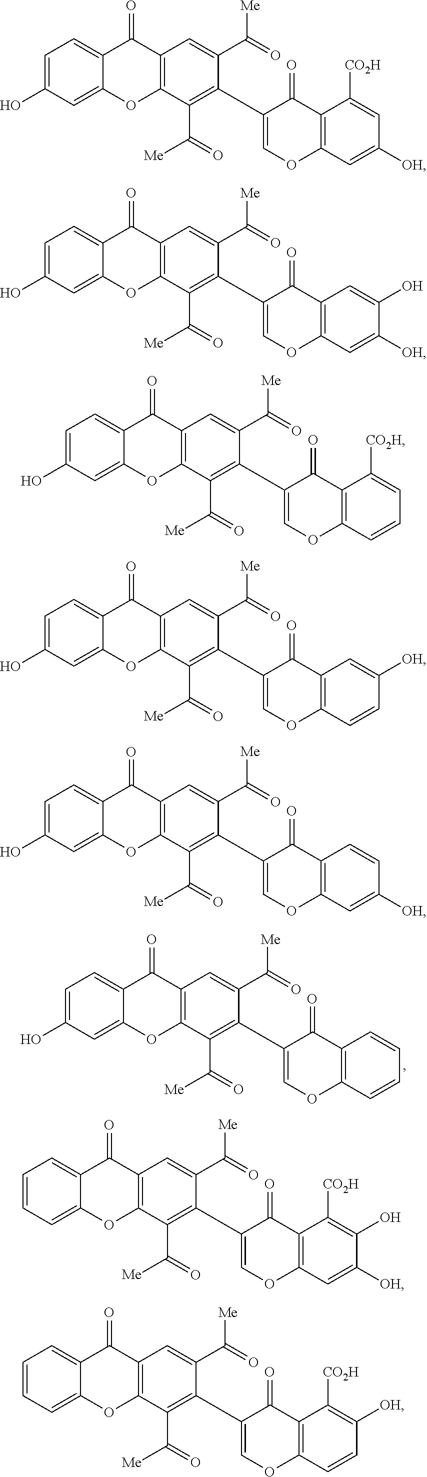

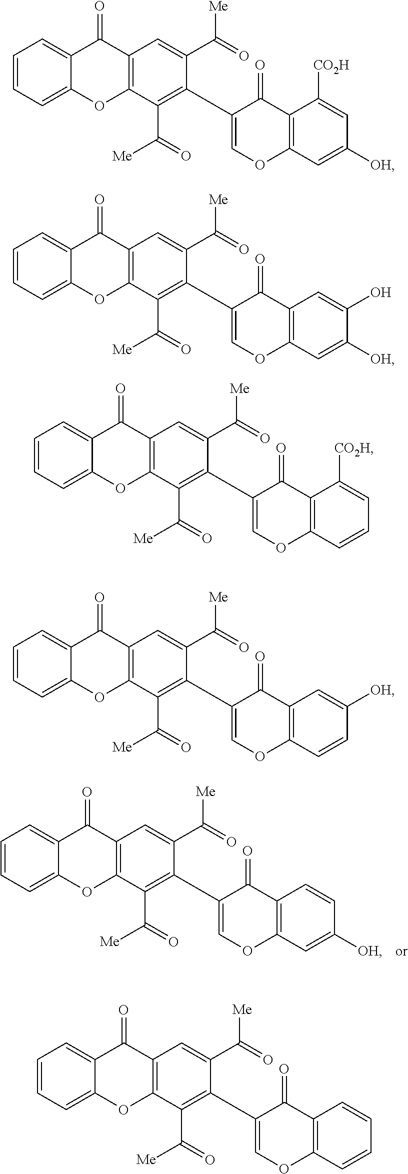

or a salt or tautomer thereof. In some embodiments, the reaction further comprises a first base. In some embodiments, the first base is a nitrogenous base. In some embodiments, the first base is an tertiary amine_{(C≦18)}. In some embodiments, the first base is a trialkylamine_{(C≦18)}. In some embodiments, the first base is triethylamine. In some embodiments, the first solvent is an organic solvent. In some embodiments, the first solvent is a substituted alkane_{(C≦8)} or amide_{(C≦8)}. In some embodiments, the first solvent is acetonitrile. In some embodiments, the reaction comprises adding from about 0.01 equivalents to about 5.0 equivalents of water relative to the compound of formula II. In some embodiments, the reaction comprises adding from about 0.1 equivalents to about 3.0 equivalents of water. In some embodiments, the reaction comprises adding about 0.5 equivalents of water. In some embodiments, the reaction comprises adding from about 1 equivalent to about 20.0 equivalents of the first base relative to the compound of formula II. In some embodiments, the reaction comprises adding from about 5.0 equivalents to about 15.0 equivalents of the first base. In some embodiments, the reaction comprises adding about 10 equivalents of the first base. In some embodiments, the reaction comprises performing the reaction at a first temperature from about 0° C. to about 80° C. In some embodiments, the first temperature is from about 0° C. to about 40° C. In some embodiments, the first temperature is about 23° C. In some embodiments, the first temperature is about room temperature. In some embodiments, the reaction comprises performing the reaction for a first time period from about 10 minutes to about 36 hours. In some embodiments, the first time period is about 10 hours to about 24 hours. In some embodiments, the first time period is about 16 hours. In some embodiments, the reaction further comprises mixing the reaction mixture. In other embodiments, the reaction comprises adding from about 100 equivalents to about 2500 equivalents of water relative to the compound of formula II. In other embodiments, the reaction comprises adding from about 500 equivalents to about 1500 equivalents of water. In other embodiments, the reaction comprises adding about 1000 equivalents of water. In other embodiments, the reaction comprises performing the reaction for a first time period from about 10 minutes to about 6 hours. In other embodiments, the first time period is about 30 minutes to about 4 hours. In other embodiments, the first time period is about 1 hour. In other embodiments, the method further comprises removing the solvent in vacao. In other embodiments, wherein the method further comprises drying the reaction using sodium sulfate. In other embodiments, the method further comprises adding after a first time period a compound of the formula:

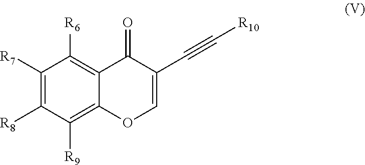

wherein: R₆, R₇, R₈, and R₉ are each independently hydrogen, amino, carboxy, halo, hydroxy, mercapto, or alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅, wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and R₁₀ is hydrogen, acyl_{(C≦12)}, or substituted acyl_{(C≦12)}; to a second solvent and reacting for a second time period. In some embodiments, the method further comprises adding a second base. In some embodiments, the base is a nitrogenous base. In some embodiments, the base is an tertiary amine_{(C≦18)}. In some embodiments, the base is a trialkylamine_{(C≦18)}. In some embodiments, the base is triethylamine. In some embodiments, the reaction comprises adding from about 0.1 equivalents to about 3.0 equivalents of the compound of formula V relative to the compound of formula II. In some embodiments, the reaction comprises adding from about 0.5 equivalents to about 2.0 equivalents of the compound of formula V. In some embodiments, the reaction comprises adding about 1.0 equivalents of the compound of formula V. In some embodiments, the reaction comprises adding from about 0.1 equivalents to about 3.0 equivalents of the second base relative to the compound of formula II. In some embodiments, the reaction comprises adding from about 0.5 equivalents to about 2.0 equivalents of the second base. In some embodiments, the reaction comprises adding about 1.0 equivalents of the second base. In some embodiments, the second solvent is an organic solvent. In some embodiments, the second solvent is a substituted alkane_{(C≦8)} or amide_{(C≦8)}. In some embodiments, the second solvent is acetonitrile. In some embodiments, the reaction comprises performing the reaction at a second temperature from about 0° C. to about 80° C. In some embodiments, the second temperature is from about 0° C. to about 40° C. In some embodiments, second temperature is about 23° C. In some embodiments, the second temperature is about room temperature. In some embodiments, the reaction comprises performing the reaction for a second time period from about 10 minutes to about 36 hours. In some embodiments, the second time period is about 10 hours to about 24 hours. In some embodiments, the second time period is about 16 hours. In some embodiments, the reaction further comprises mixing the compound of formula II, the compound of formula V, and the second base in the second solvent. In some embodiments, the reaction has a yield of greater than 25%. In some embodiments, the yield is greater than 50%. In some embodiments, the yield is greater than 70%.

In another aspect, the present disclosure provides a method of preparing a compound of the formula:

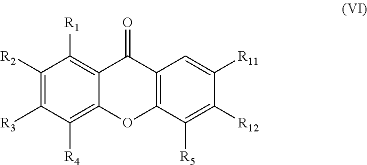

wherein: R₁, R₂, R₃, and R₄ are each independently hydrogen, amino, carboxy, halo, hydroxy, mercapto, or alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅, wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; R₅ is hydrogen, acyl_{(C≦12)}, or substituted acyl_{(C≦12)}; and R₁₁ and R₁₂ are each independently alkyl_{(C≦12)}, aryl_{(C≦12)}, acyl_{(C≦12)}, or a substituted version of any of these groups; or a salt thereof; comprising reacting a compound of the formula:

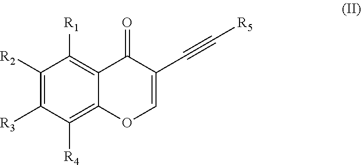

wherein: R₁, R₂, R₃, and R₄ are each independently hydrogen, amino, carboxy, halo, hydroxy, mercapto, or alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅, wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and R₅ is hydrogen, acyl_{(C≦12)}, or substituted acyl_{(C≦12)}; with a compound of the formula:

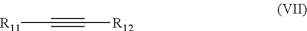

wherein: R₁₁ and R₁₂ are each independently alkyl_{(C≦12)}, aryl_{(C≦12)}, acyl_{(C≦12)}, or a substituted version of any of these groups; in the presence of a base and water in a solvent.

In yet another aspect, the present disclosure provides a method of preparing a compound of the formula:

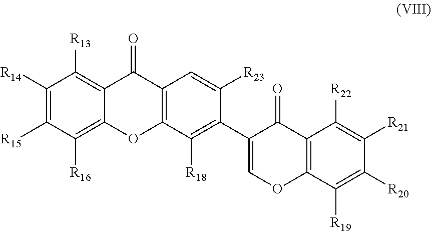

wherein: R₁₃, R₁₄, R₁₅, R₁₆, R₁₉, R₂₀, R₂₁, and R₂₂ are each independently hydrogen, amino, carboxy, halo, hydroxy, mercapto, or alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅, wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and R₁₈ and R₂₃ are each independently acyl_{(C≦18)} or substituted acyl_{(C≦18)}; or a salt thereof; comprising
A) reacting a compound of the formula:

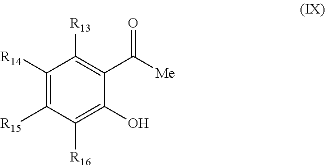

wherein: R₁₃, R₁₄, R₁₅, and R₁₆ are as defined above; with Me₂NCH(OMe)₂ in the presence of a solvent to form a compound of the formula:

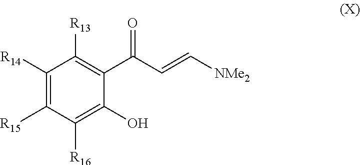

wherein: R₁₃, R₁₄, R₁₅, and R₁₆ are as defined above;
B) reacting the compound of formula X with iodide in a solvent to form a compound of the formula:

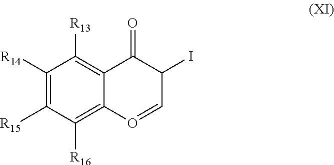

wherein: R₁₃, R₁₄, R₁₅, and R₁₆ are as defined above;
C) reacting the compound of formula X with a compound of the formula:

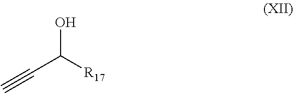

wherein: R₁₇ is hydrogen, alkyl_{(C≦17)}, cycloalkyl_{(C≦17)}, alkenyl_{(C≦17)}, alkynyl_{(C≦17)}, aryl_{(C≦17)}, aralkyl_{(C≦17)}, heteroaryl_{(C≦17)}, heteroaralkyl_{(C≦17)}, heterocycloalkyl_{(C≦17)}, or a substituted version of any of these groups; in the presence of a transition metal catalyst and a base in a solvent to form a compound of the formula:

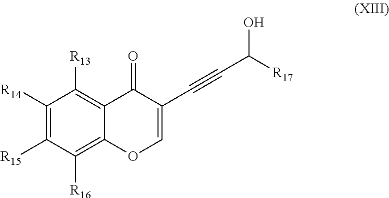

wherein: R₁₃, R₁₄, R₁₅, R₁₆, and R₁₇ are as defined above;
D) reacting the compound of formula XIII with an oxidizing agent in a solvent to form a compound of the formula:

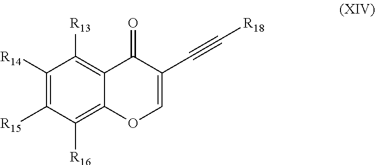

wherein: R₁₃, R₁₄, R₁₅, and R₁₆ are as defined above; and R₁₈ is acyl_{(C≦18)} or substituted acyl_{(C≦18)}; and
E) reacting the compound of formula XIV with a base and water in a solvent to form the compound of formula VIII wherein: R₁₃ and R₁₉, R₁₄ and R₂₀, R₁₅ and R₂₁, R₁₆ and R₂₂, and R₁₈ and R₂₃ are the same and as defined above; or
F) reacting the compound of formula XIV with a compound of the formula:

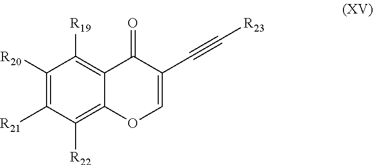

wherein: R₁₉, R₂₀, R₂₁, R₂₂, and R₂₃ are as defined above; in the presence of a base and water in a solvent to form the compound of formula VIII. In some embodiments, R₁₃ is hydrogen, carboxy, hydroxy, or alkyl_{(C≦12)}, acyl_{(C≦12)}, or a substituted version of any of these groups, or —OX₁ or —C(O)OX₅, wherein: X₁ is a hydroxy protecting group and X₅ is a carboxy protecting group. In some embodiments, R₁₄ is hydrogen, carboxy, hydroxy, or alkyl_{(C≦12)}, acyl_{(C≦12)}, or a substituted version of any of these groups, or —OX₁ or —C(O)OX₅, wherein: X₁ is a hydroxy protecting group and X₅ is a carboxy protecting group. In some embodiments, R₁₅ is hydrogen, carboxy, hydroxy, or alkyl_{(C≦12)}, acyl_{(C≦12)}, or a substituted version of any of these groups, or —OX₁ or —C(O)OX₅, wherein: X₁ is a hydroxy protecting group and X₅ is a carboxy protecting group. In some embodiments, R₁₆ is hydrogen, carboxy, hydroxy, or alkyl_{(C≦12)}, acyl_{(C≦12)}, or a substituted version of any of these groups, or —OX₁ or —C(O)OX₅, wherein: X₁ is a hydroxy protecting group and X₅ is a carboxy protecting group. In some embodiments, R₁₉ is hydrogen, carboxy, hydroxy, or alkyl_{(C≦12)}, acyl_{(C≦12)}, or a substituted version of any of these groups, or —OX₁ or —C(O)OX₅, wherein: X₁ is a hydroxy protecting group and X₅ is a carboxy protecting group. In some embodiments, R₂₀ is hydrogen, carboxy, hydroxy, or alkyl_{(C≦12)}, acyl_{(C≦12)}, or a substituted version of any of these groups, or —OX₁ or —C(O)OX₅, wherein: X₁ is a hydroxy protecting group and X₅ is a carboxy protecting group. In some embodiments, R₂₁ is hydrogen, carboxy, hydroxy, or alkyl_{(C≦12)}, acyl_{(C≦12)}, or a substituted version of any of these groups, or —OX₁ or —C(O)OX₅, wherein: X₁ is a hydroxy protecting group and X₅ is a carboxy protecting group. In some embodiments, R₂₂ is hydrogen, carboxy, hydroxy, alkyl_{(C≦12)}, acyl_{(C≦12)}, substituted alkyl_{(C≦12)}, substituted acyl_{(C≦12)}, or —OX₁ or —C(O)OX₅, wherein: X₁ is a hydroxy protecting group and X₅ is a carboxy protecting group. In some embodiments, R₁₇ is hydrogen, alkyl_{(C≦17)}, cycloalkyl_{(C≦17)}, aryl_{(C≦17)}, aralkyl_{(C≦17)}, heteroaryl_{(C≦17)}, heteroaralkyl_{(C≦17)}, or a substituted version of any of these groups. In some embodiments, R₁₇ is hydrogen, alkyl_{(C≦17)}, cycloalkyl_{(C≦17)}, aryl_{(C≦17)}, substituted alkyl_{(C≦17)}, substituted cycloalkyl_{(C≦17)}, or substituted aryl_{(C≦17)}. In some embodiments, the reaction of step A) comprises adding from about 1.0 equivalents to about 10.0 equivalents of Me₂NCH(OMe)₂ relative to the compound of formula IX. In some embodiments, the reaction of step A) comprises adding from about 2.0 equivalents to about 8.0 equivalents of Me₂NCH(OMe)₂. In some embodiments, the reaction of step A) comprises adding about 5.0 equivalents of Me₂NCH(OMe)₂. In some embodiments, the solvent of step A) is a substituted alkane_{(C≦8)}. In some embodiments, the solvent of step A) is dimethoxyethane. In some embodiments, the reaction of step A) comprises performing the reaction at a temperature from about 50° C. to about 120° C. In some embodiments, the temperature is from about 60° C. to about 100° C. In some embodiments, the temperature is about 85° C. In some embodiments, the reaction of step A) comprises performing the reaction for a time period from about 1 hour to about 12 hours. In some embodiments, the time period is about 2 hours to about 6 hours. In some embodiments, the time period is about 4 hours. In some embodiments, the reaction of step A) further comprises mixing the compound of formula IX and Me₂NCH(OMe)₂ in the solvent. In some embodiments, the reaction of step B) comprises adding from about 0.5 equivalents to about 5.0 equivalents of I₂ relative to the compound of formula X. In some embodiments, the reaction of step B) comprises adding from about 1.0 equivalent to about 3.0 equivalents of I₂. In some embodiments, the reaction of step B) comprises adding about 2.0 equivalents of I₂. In some embodiments, the solvent of step B) is a substituted alkane_{(C≦8)}. In some embodiments, the solvent of step B) is chloroform. In some embodiments, the reaction of step B) comprises performing the reaction at a temperature from about 0° C. to about 50° C. In some embodiments, the temperature is from about 15° C. to about 30° C. In some embodiments, the temperature is about 23° C. In some embodiments, the temperature is room temperature. In some embodiments, the reaction of step B) comprises performing the reaction for a time period from about 15 minutes to about 4 hours. In some embodiments, the time period is about 30 minutes to about 2 hours. In some embodiments, the time period is about 1 hour. In some embodiments, the reaction of step B) further comprises mixing the compound of formula X and I₂ in the solvent. In some embodiments, the transition metal catalyst of step C) is a palladium catalyst. In some embodiments, the transition metal catalyst is a palladium(II) catalyst. In some embodiments, the transition metal catalyst is bis(triphenylphosphine) palladium(II) dichloride. In some embodiments, the reaction of step C) comprises adding from about 0.001 equivalents to about 1.0 equivalent of the transition metal catalyst relative to the compound of formula XI. In some embodiments, the reaction of step C) comprises adding from about 0.01 equivalent to about 0.5 equivalents of the transition metal catalyst. In some embodiments, the reaction of step C) comprises adding about 0.02 equivalents of the transition metal catalyst. In some embodiments, the transition metal catalyst of step C) further comprises a second metal salt. In some embodiments, the second metal salt is a copper salt. In some embodiments, the second metal salt is a copper(I) salt. In some embodiments, the second metal salt is copper(I) iodide. In some embodiments, the reaction of step C) comprises adding from about 0.001 equivalents to about 2.0 equivalents of the second metal salt relative to the compound of formula XI. In some embodiments, the reaction of step C) comprises adding from about 0.01 equivalent to about 0.5 equivalents of the second metal salt. In some embodiments, the reaction of step C) comprises adding about 0.1 equivalents of the second metal salt. In some embodiments, the base of step C) is a nitrogenous base. In some embodiments, the base is a trialkylamine_{(C≦18)}. In some embodiments, the base is diisopropylamine. In some embodiments, the reaction of step C) comprises adding from about 1.0 equivalent to about 10.0 equivalents of the base relative to the compound of formula XI. In some embodiments, the reaction of step C) comprises adding from about 2.0 equivalents to about 5.0 equivalents of the base. In some embodiments, the reaction of step C) comprises adding about 3.0 equivalents of the base. In some embodiments, the reaction of step C) comprises adding from about 1.0 equivalent to about 10.0 equivalents of the compound of formula XII relative to the compound of formula XI. In some embodiments, the reaction of step C) comprises adding from about 2.0 equivalents to about 6.0 equivalents of the compound of formula XII. In some embodiments, the reaction of step C) comprises adding about 4.0 equivalents of the compound of formula XII. In some embodiments, the solvent of step C) is an ether_{(C≦8)} or substituted ether_{(C≦8)}. In some embodiments, the solvent of step C) is tetrahydrofuran. In some embodiments, the reaction of step C) comprises performing the reaction at a temperature from about 0° C. to about 50° C. In some embodiments, the temperature is from about 15° C. to about 30° C. In some embodiments, the temperature is about 23° C. In some embodiments, the temperature is room temperature. In some embodiments, the reaction of step C) comprises performing the reaction for a time period from about 15 minutes to about 4 hours. In some embodiments, the time period is about 30 minutes to about 2 hours. In some embodiments, the time period is about 1 hour. In some embodiments, the reaction further comprises mixing the compound of formula XI, the compound of formula XII, the base, the transition metal catalyst, and the second metal salt in the solvent. In some embodiments, the oxidizing agent of step D) is a chromic compound. In some embodiments, the oxidizing agent is pyridinium dichromate. In some embodiments, the reaction of step D) comprises adding from about 1.0 equivalent to about 10.0 equivalents of the oxidizing agent relative to the compound of formula X. In some embodiments, the reaction of step D) comprises adding from about 2.0 equivalents to about 8.0 equivalents of the oxidizing agent. In some embodiments, the reaction of step D) comprises adding about 5.0 equivalents of the oxidizing agent. In some embodiments, the solvent of step D) is a substituted alkane_{(C≦8)}. In some embodiments, the solvent of step D) is dichloromethane. In some embodiments, the reaction of step D) comprises performing the reaction at a temperature from about 0° C. to about 50° C. In some embodiments, the temperature is from about 15° C. to about 30° C. In some embodiments, the temperature is about 23° C. In some embodiments, the temperature is room temperature. In some embodiments, the reaction of step D) comprises performing the reaction for a time period from about 1 hour to about 10 hours. In some embodiments, the time period is about 2 hours to about 8 hours. In some embodiments, the time period is about 5 hour. In some embodiments, the reaction of step D) further comprises adding 4.0 Å molecular sieves. In some embodiments, the reaction of step D) further comprises mixing the compound of formula XIII, the oxidizing agent, and the molecular sieves in the solvent. In some embodiments, one or more steps of the reaction further comprises a deprotection step to remove one or more protecting groups. In some embodiments, one or more steps of the reaction further comprises a purification step. In some embodiments, the purification step comprises purifying the reaction such that the desired compound comprises greater than 90% of the total mass. In some embodiments, the purification step comprises purifying the reaction such that the compound comprises greater than 95% of the total mass. In some embodiments, the purification step comprises purifying the reaction via extraction or chromatography. In some embodiments, the chromatography is column chromatography. In some embodiments, the column chromatography is silica gel or alumina column chromatography.

In still another aspect, the present disclosure provides a method for treating a disease or disorder comprising modulating the activity of a G-coupled protein receptor comprising administering to a patient in need thereof a therapeutically effective amount of a compound of the formula:

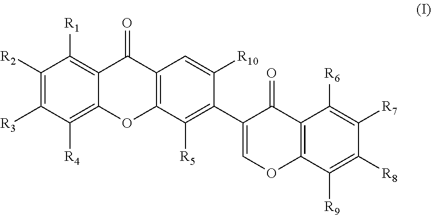

wherein: R₁, R₂, R₃, R₄, R₆, R₇, R₈, and R₉ are each independently hydrogen, amino, carboxy, halo, hydroxy, mercapto, or alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅, wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and R₅ and R₁₀ are each independently hydrogen, acyl_{(C≦12)}, or substituted acyl_{(C≦12)}; or a compound of the formula:

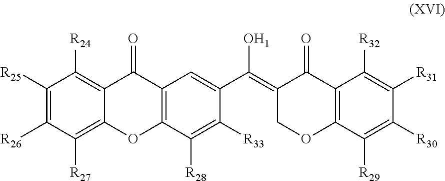

wherein: R₂₄, R₂₅, R₂₆, R₂₇, R₂₉, R₃₀, R₃₁, and R₃₂ are each independently hydrogen, amino, carboxy, halo, hydroxy, mercapto, or alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅, wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; R₂₈ is hydrogen, acyl_{(C≦12)}, or substituted acyl_{(C≦12)}; and R₃₃ is hydrogen, alkyl_{(C≦12)}, substituted alkyl_{(C≦12)}, acyl_{(C≦12)}, or substituted acyl_{(C≦12)}; or a pharmaceutically acceptable salt or tautomer thereof. In some embodiments, the G-coupled protein receptor is a succinate receptor. In some embodiments, the succinate receptor is G-coupled protein receptor succinate receptor 1. In some embodiments, the disease or disorder is excessive angiogenesis of the retina or cornea. In some embodiments, the disease or disorder is retinopathy. In some embodiments, the retinopathy is caused by excessive angiogenesis of the retina and cornea. In some embodiments, the disease or disorder is an infection. In some embodiments, treating the infection comprises activating a dendritic cell. In some embodiments, the disease or disorder is cancer. In some embodiments, the cancer is a carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In some embodiments, the compound is administered orally, intravenously, topically, intraocularly, or locally. In some embodiments, the method further comprises a second therapeutic agent. In some embodiments, the second therapeutic agent is succinic acid or a salt thereof, a chemotherapeutic, surgery, an immunotherapy, a genetic therapy, an antibiotic, or an anti-viral agent.

In another aspect, the present disclosure provides a method of treating a disease or disorder associate with inflammation or vascular proliferation comprising administering a patient in need thereof a therapeutically effective amount of a compound of the formula:

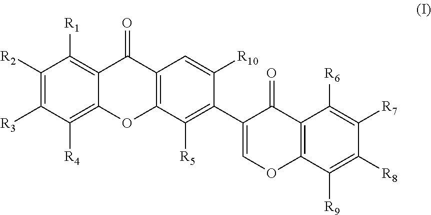

wherein: R₁, R₂, R₃, R₄, R₆, R₇, R₈, and R₉ are each independently hydrogen, amino, carboxy, halo, hydroxy, mercapto, or alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅, wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and R₅ and R₁₀ are each independently hydrogen, acyl_{(C≦12)}, or substituted acyl_{(C≦12)}; or a compound of the formula:

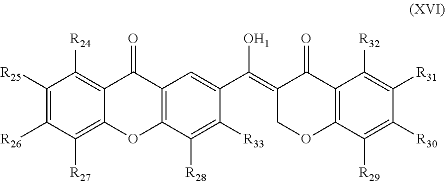

wherein: R₂₄, R₂₅, R₂₆, R₂₇, R₂₉, R₃₀, R₃₁, and R₃₂ are each independently hydrogen, amino, carboxy, halo, hydroxy, mercapto, or alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅, wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; R₂₈ is hydrogen, acyl_{(C≦12)}, or substituted acyl_{(C≦12)}; and R₃₃ is hydrogen, alkyl_{(C≦12)}, substituted alkyl_{(C≦12)}, acyl_{(C≦12)}, or substituted acyl_{(C≦12)}; or a pharmaceutically acceptable salt or tautomer thereof. In some embodiments, the disease or disorder is a cardiovascular disease or disorder, a dermatological disease or disorder, a metabolic disease or disorder, cancer, a gastrointestinal or liver disease or disorder, a hematological disease or disorder, a reproductive disease or disorder, an endocrinal disease or disorder, an inflammatory disease or disorder, a muscle-skeleton disease or disorder, a neurological disease or disorder, a urological disease or disorder, a respiratory disease or disorder, and an ophthalmological disease or disorder. In some embodiments, the disease or disorder is cancer, diabetic retinopathy, or an infection. In some embodiments, the disease or disorder is associated with dysregulation of a G-coupled protein receptor. In some embodiments, the G-coupled protein receptor is a succinate receptor. In some embodiments, the G-coupled protein receptor is G-coupled protein receptor succinate receptor 1. In some embodiments, the compound acts as an agonist of G-coupled protein receptor succinate receptor 1. In some embodiments, the compound acts as an antagonist of G-coupled protein receptor succinate receptor 1. In some embodiments, the method further comprises a second therapeutic agent. In some embodiments, the second therapeutic agent is succinic acid or a salt thereof, a chemotherapeutic, surgery, an immunotherapy, a genetic therapy, an antibiotic, or an anti-viral agent.

In another aspect, the present disclosure provides a method of promoting nerve regeneration comprising administering to a patient in need thereof a therapeutically effective amount of succinic acid or a salt thereof and a compound of the formula:

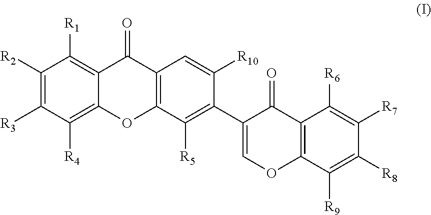

wherein: R₁, R₂, R₃, R₄, R₆, R₇, R₈, and R₉ are each independently hydrogen, amino, carboxy, halo, hydroxy, mercapto, or alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅, wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and R₅ and R₁₀ are each independently hydrogen, acyl_{(C≦12)}, or substituted acyl_{(C≦12)}; or a compound of the formula:

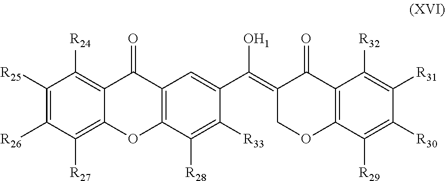

wherein: R₂₄, R₂₅, R₂₆, R₂₇, R₂₉, R₃₀, R₃₁, and R₃₂ are each independently hydrogen, amino, carboxy, halo, hydroxy, mercapto, or alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅, wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; R₂₈ is hydrogen, acyl_{(C≦12)}, or substituted acyl_{(C≦12)}; and R₃₃ is hydrogen, alkyl_{(C≦12)}, substituted alkyl_{(C≦12)}, acyl_{(C≦12)}, or substituted acyl_{(C≦12)}; or a pharmaceutically acceptable salt or tautomer thereof. In some embodiments, the method comprised contacting a nerve of the central nervous system, the peripheral nervous system or both with the compound. In some embodiments, the succinate salt is sodium succinate. In some embodiments, the method leads to axonal regeneration. In some embodiments, the method leads to axonal myelination. In some embodiments, the method promotes angiogenesis. In some embodiments, the method promotes cellular survival. In some embodiments, the method comprises modulating the activity of G-coupled protein receptor succinate receptor 1. In some embodiments, the composition promotes neural regeneration modulates the effects of a disease or disorder. In some embodiments, the neural regeneration mitigates the effects of a spinal cord injury. In some embodiments, the neural regeneration mitigates the effects of a disease or disorder. In some embodiments, the disease or disorder is a neurological disease or disorder. In some embodiments, the neurological disease or disorder is Alzheimer's disease or Parkinson's disease. In some embodiments, the method further comprises a second therapeutic agent.

In yet another aspect, the present disclosure provides a compound of the formula:

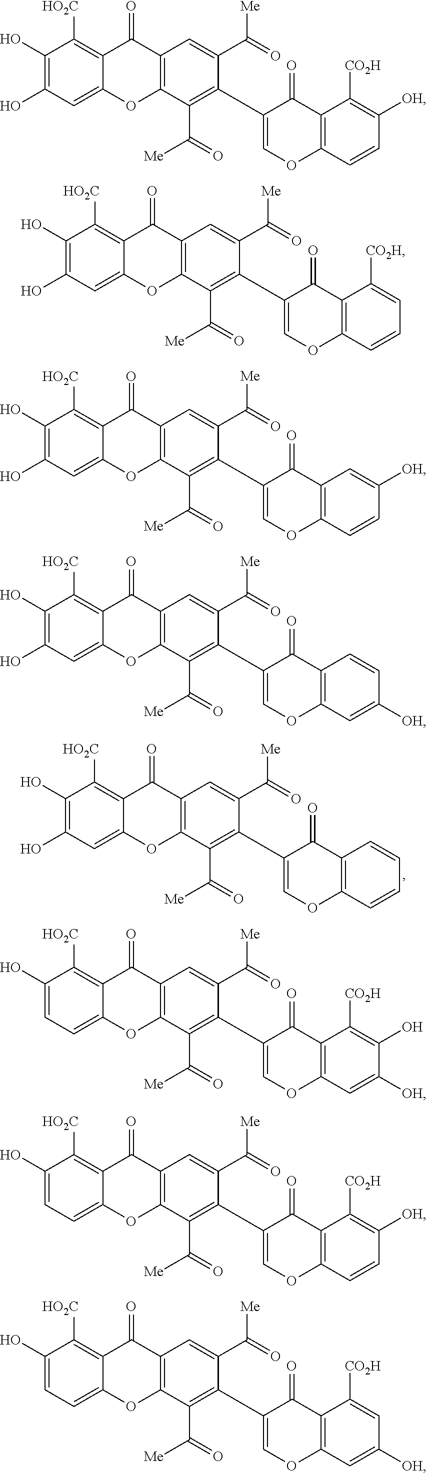

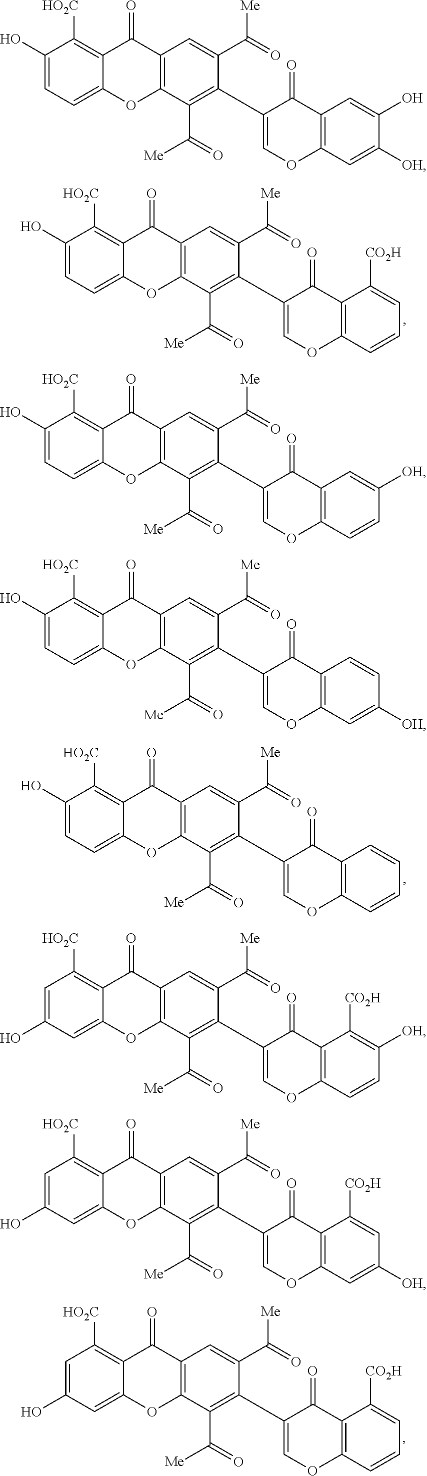

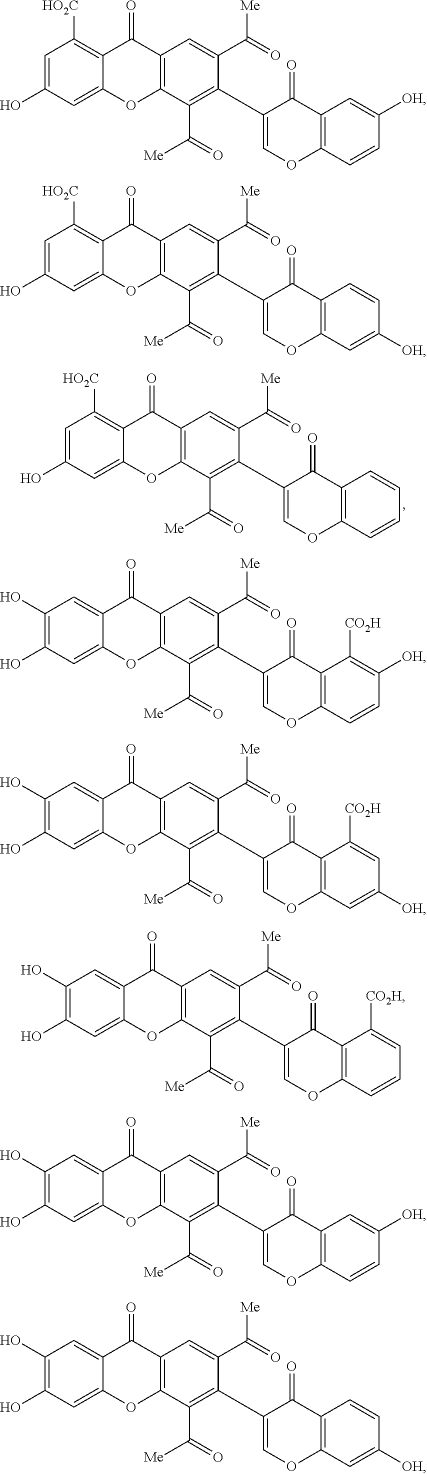

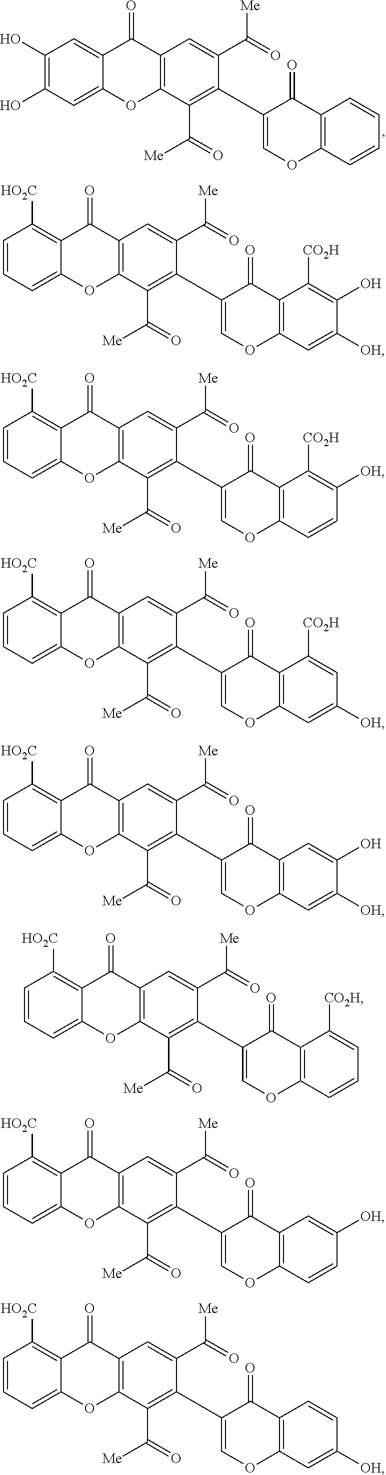

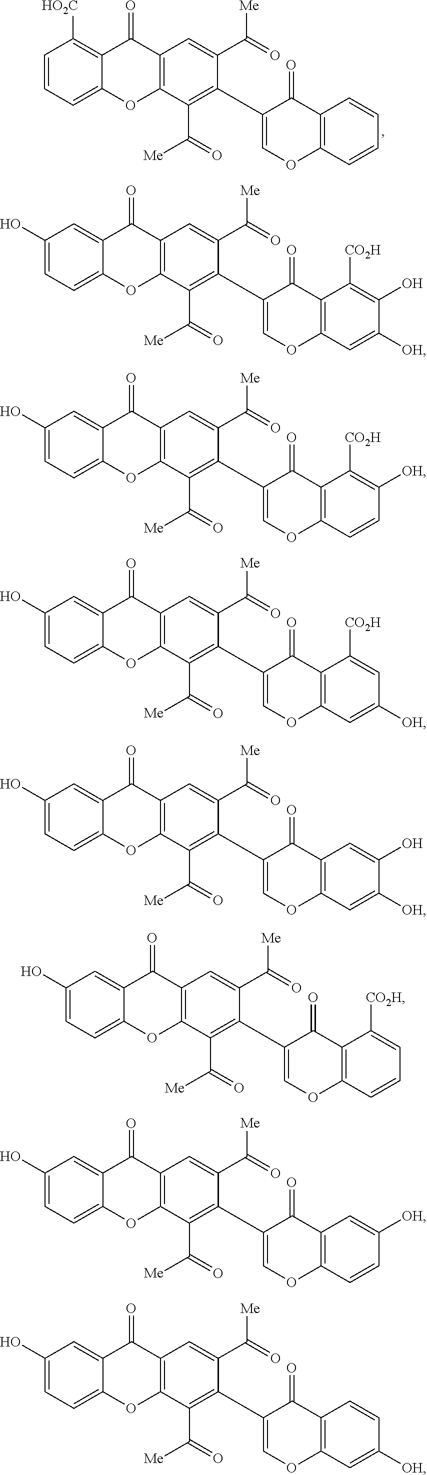

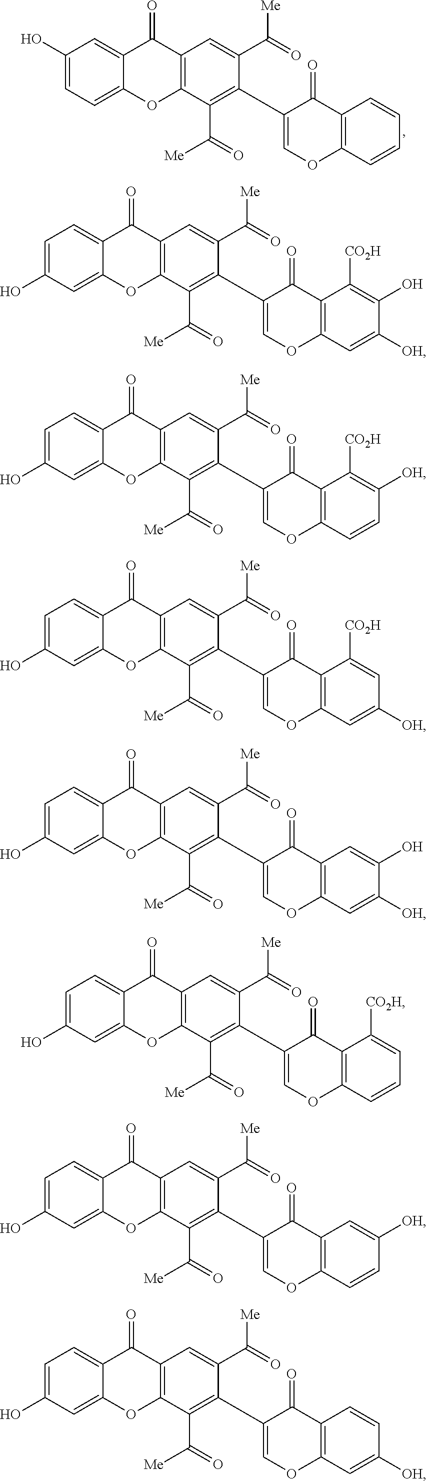

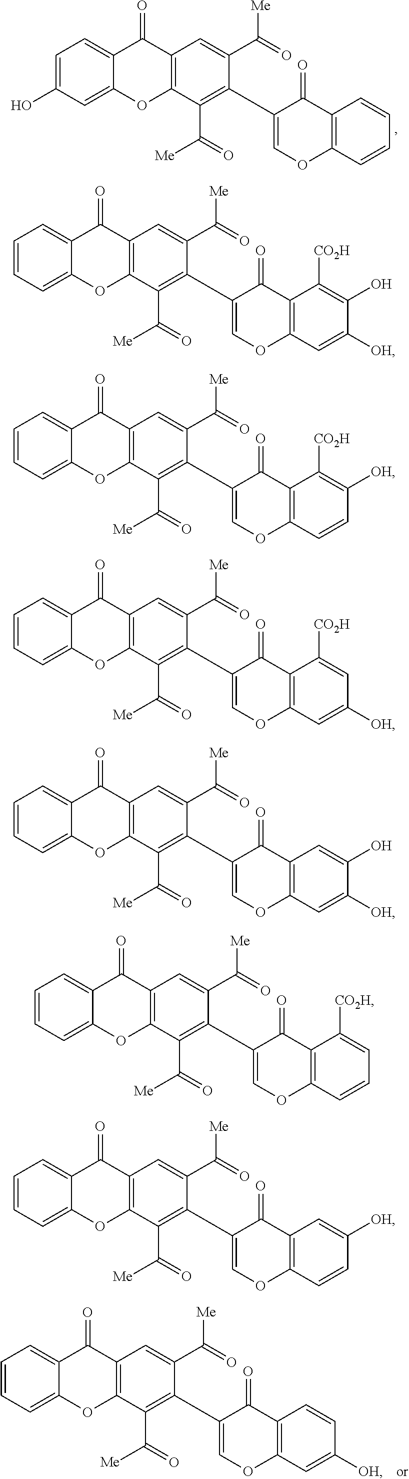

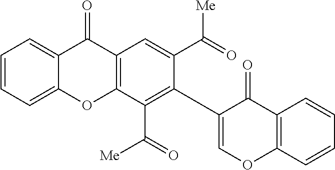 [SMILES: CC(=O)c1cc2c(=O)c3ccccc3oc2c(C(C)=O)c1-c1coc2ccccc2c1=O]

or a pharmaceutically acceptable salt or tautomer thereof.

In another aspect, the present disclosure provides a pharmaceutical composition comprising a compound of the present disclosure and a pharmaceutically acceptable excipient. In some embodiments, the composition is formulated for administration locally, orally, systemically, intravenously, topically, or intraocularly. In some embodiments, the composition is formulated in a fixed dose form.

In still another aspect, the present disclosure provides a composition for use in treating a disease or disorder comprising modulating the activity of a G-coupled protein receptor, a composition for use in treating a disease or disorder associate with inflammation or vascular proliferation, or a composition for use in promoting nerve regeneration comprising succinic acid or a salt thereof. In some embodiments, the composition further comprises a compound of the formula:

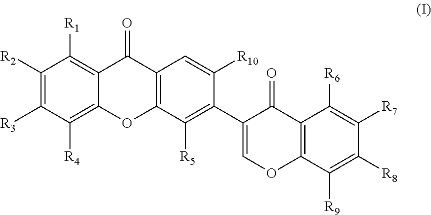

wherein: R₁, R₂, R₃, R₄, R₆, R₇, R₈, and R₉ are each independently hydrogen, amino, carboxy, halo, hydroxy, mercapto, or alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅, wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and R₅ and R₁₀ are each independently hydrogen, acyl_{(C≦12)}, or substituted acyl_{(C≦12)}; or a compound of the formula:

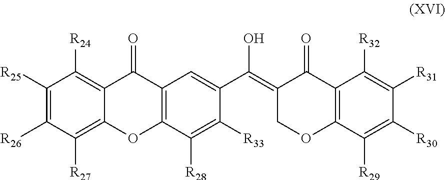

wherein: R₂₄, R₂₅, R₂₆, R₂₇, R₂₉, R₃₀, R₃₁, and R₃₂ are each independently hydrogen, amino, carboxy, halo, hydroxy, mercapto, or alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅, wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; R₂₈ is hydrogen, acyl_{(C≦12)}, or substituted acyl_{(C≦12)}; and R₃₃ is hydrogen, alkyl_{(C≦12)}, substituted alkyl_{(C≦12)}, acyl_{(C≦12)}, or substituted acyl_{(C≦12)}; or a pharmaceutically acceptable salt or tautomer thereof.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula does not mean that it cannot also belong to another generic formula.

### BRIEF DESCRIPTION OF THE DRAWINGS

The following drawings form part of the present specification and are included to further demonstrate certain aspects of the present disclosure. The invention may be better understood by reference to one of these drawings in combination with the detailed description of specific embodiments presented herein.

FIG. 1: shows a bar graph of the outgrowth activity of vinaxanthone and xanthofulvin compared to dibutyryl cAMP in vivo.

FIG. 2: shows the modulation of different vinaxanthone and xanthofulvin analogs of SUCNR1. The values are compared to 100% activation by sodium succinate.

FIG. 3: shows the increased efficacy of the compounds when administered with sodium succinate versus the addition of succinate.

### DESCRIPTION OF ILLUSTRATIVE EMBODIMENTS

In some aspects of the present invention, analogs of vinaxanthone and xanthofulvin are presented. The analogs of the present disclosure may be used as either an antagonist or agonist of the G-coupled protein receptors succinate receptor 1. The present disclosure provides modular synthesis methods for prepare vinaxanthone and analogs thereof. The present disclosure also provides a method of treating a disease or disorder using the compounds described herein. Additionally, the present disclosure provides methods of treatment using the compounds of the present disclosure and succinic acid or a salt thereof for a variety of disease including spinal cord injury or neural regeneration.

### I. DEFINITIONS

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “=═” means a double bond, and “≡” means triple bond. The symbol “----” represents an optional bond, which if present is either single or double. The symbol “ [symbol] ” represents a single bond or a double bond. Thus, for example, the formula

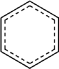

includes

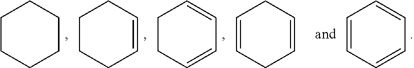

And it is understood that no one such ring atom forms part of more than one double bond. Furthermore, it is noted that the covalent bond symbol “—”, when connecting one or two stereogenic atoms, does not indicate any preferred stereochemistry. Instead, it cover all stereoisomers as well as mixtures thereof. The symbol “ [symbol] ”, when drawn perpendicularly across a bond (e.g.,

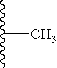

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

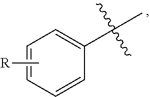

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

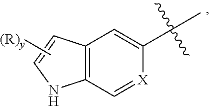

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the groups and classes below, the number of carbon atoms in the group is as indicated as follows: “Cn” defines the exact number (n) of carbon atoms in the group/class. “C≦n” defines the maximum number (n) of carbon atoms that can be in the group/class, with the minimum number as small as possible for the group in question, e.g., it is understood that the minimum number of carbon atoms in the group “alkenyl_{(C≦8)}” or the class “alkene_{(C≦8)}” is two. Compare with “alkoxy_{(C=10)}”, which designates alkoxy groups having from 1 to 10 carbon atoms. Also compare “phosphine_{(C=10)}”, which designates phosphine groups having from 0 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. Typically the carbon number indicator follows the group it modifies, is enclosed with parentheses, and is written entirely in subscript; however, the indicator may also precede the group, or be written without parentheses, without signifying any change in meaning. Thus, the terms “C5 olefin”, “C5-olefin”, “olefin_{(C5)}”, and “olefin_{C5}” are all synonymous.

The term “saturated” as used herein means the compound or group so modified has no carbon-carbon double and no carbon-carbon triple bonds, except as noted below. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded.

The term “aliphatic” when used without the “substituted” modifier signifies that the compound/group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl).

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the compound H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups.

The term “cycloalkyl” when used without the “substituted” modifier refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). The term “cycloalkanediyl” when used without the “substituted” modifier refers to a divalent saturated aliphatic group with two carbon atoms as points of attachment, no carbon-carbon double or triple bonds, and no atoms other than carbon and hydrogen. The group

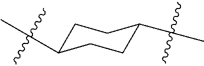 [SMILES: *C1CCC(*)CC1]

is a non-limiting example of cycloalkanediyl group. A “cycloalkane” refers to the compound H—R, wherein R is cycloalkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The term “alkenyl” when used without the “substituted” modifier refers to an monovalent unsaturated aliphatic group with a carbon atom as the point of attachment, a linear or branched acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” or “olefin” are synonymous and refer to a compound having the formula H—R, wherein R is alkenyl as this term is defined above. A “terminal alkene” refers to an alkene having just one carbon-carbon double bond, wherein that bond forms a vinyl group at one end of the molecule. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The groups —CH═CHF, —CH═CHCl and —CH═CHBr are non-limiting examples of substituted alkenyl groups.

The term “alkynyl” when used without the “substituted” modifier refers to an monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the compound H—R, wherein R is alkynyl. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

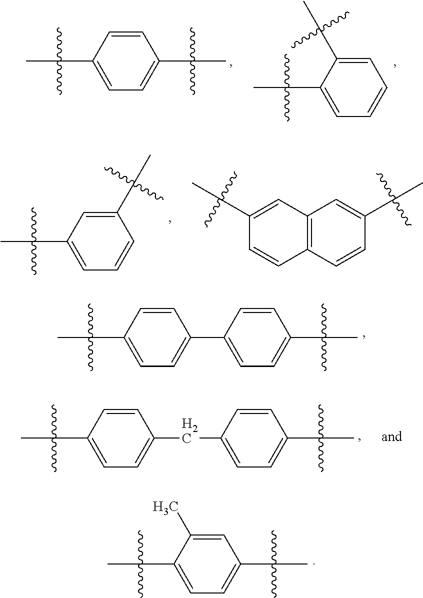

An “arene” refers to the compound H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl, pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the compound H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The term “heterocycloalkyl” when used without the “substituted” modifier refers to a monovalent non-aromatic group with a carbon atom or nitrogen atom as the point of attachment, said carbon atom or nitrogen atom forming part of one or more non-aromatic ring structures wherein at least one of the ring atoms is nitrogen, oxygen or sulfur, and wherein the heterocycloalkyl group consists of no atoms other than carbon, hydrogen, nitrogen, oxygen and sulfur. If more than one ring is present, the rings may be fused or unfused. As used herein, the term does not preclude the presence of one or more alkyl groups (carbon number limitation permitting) attached to the ring or ring system. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, —S(O)₂NH₂, or —C(O)OC(CH₃)₃ (tert-butyloxycarbonyl, BOC).

The term “acyl” when used without the “substituted” modifier refers to the group —C(O)R, in which R is a hydrogen, alkyl, cycloalkyl, alkenyl, aryl, aralkyl or heteroaryl, as those terms are defined above. The groups, —CHO, —C(O)CH₃ (acetyl, Ac), —C(O)CH₂CH₃, —C(O)CH₂CH₂CH₃, —C(O)CH(CH₃)₂, —C(O)CH(CH₂)₂, —C(O)C₆H₅, —C(O)C₆H₄CH₃, —C(O)CH₂C₆H₅, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —SH, —OCH₃, —OCH₂CH₃, —NHCH₃, —NHCH₂CH₃, —N(CH₃)₂, —OC(O)CH₃, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂, —N(CH₃)(CH₂CH₃), and N-pyrrolidinyl. The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, or —S(O)₂NH₂. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.” Throughout this application, the term “about” is used to indicate that a value includes the inherent variation of error for the device, the method being employed to determine the value, or the variation that exists among the study subjects.

A “base” in the context of this application is a compound which has a lone pair of electron that can accept a proton. Non-limiting examples of a base can include triethylamine, a metal hydroxide, a metal alkoxide, a metal hydride, or a metal alkane. An alkyllithium or organolithium is a compound of the formula alkyl_{(C≦12)}-Li. A nitrogenous base is an alkylamine, dialkylamine, trialkylamine, nitrogen containing heterocycloalkane or heteroarene wherein the base can accept a proton to form a positively charged species. For example, but not limited to, a nitrogenous base could be 4,4-dimethylpyridine, pyridine, 1,8-diazabicyclo[5.4.0]undec-7-ene, diisopropylethylamine, or triethylamine. A metal alkoxide is an alkoxy group wherein rather than the oxygen atom which was the point of connectivity has an extra electron and thus a negative charge which is charged balanced by the metal ion. For example, a metal alkoxide could be a sodium tert-butoxide or potassium methoxide.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which causes 50% inhibition of a given process. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

A “metal” in the context of this application is a transition metal or a metal of groups I or II. In some embodiments, a metal is lithium, sodium, or potassium. In other embodiments, a metal is calcium or magnesium.

An “oxidizing agent” in the context of this application is a compound which causes the oxidation of a compound by accepting an electron. Some non-limiting examples of oxidizing agent are oxygen gas, peroxides, chlorite, hypochlorite, or a chromium compound such as pyridinium chlorochromate or hydrochromic acid.

An “amine protecting group” is well understood in the art. An amine protecting group is a group which prevents the reactivity of the amine group during a reaction which modifies some other portion of the molecule and can be easily removed to generate the desired amine. Amine protecting groups can be found at least in Greene and Wuts, 1999, which is incorporated herein by reference. Some non-limiting examples of amino protecting groups include formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Additionally, the “amine protecting group” can be a divalent protecting group such that both hydrogen atoms on a primary amine are replaced with a single protecting group. In such a situation the amine protecting group can be phthalimide (phth) or a substituted derivative thereof wherein the term “substituted” is as defined above.

A “carboxy protecting group” is well understood in the art. A carboxy protecting group is a group which prevents the reactivity of the carboxy group during a reaction which modifies some other portion of the molecule and can be easily removed to generate the desired hydroxyl. Carboxy protecting groups can be found at least in Greene and Wuts, 1999, which is incorporated herein by reference. Some non-limiting examples or carboxy protecting groups include alkyl groups such as methyl, ethyl, or tert-butyl, aralkyl groups such as benzyl or 4-methoxybenzyl, silyl ester such as trimethylsilyl, or oxazoline groups or a substituted version of any of these groups.

A “hydroxyl protecting group” is well understood in the art. A hydroxyl protecting group is a group which prevents the reactivity of the hydroxyl group during a reaction which modifies some other portion of the molecule and can be easily removed to generate the desired hydroxyl. Hydroxyl protecting groups can be found at least in Greene and Wuts, 1999, which is incorporated herein by reference. Some non-limiting examples of hydroxyl protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like.

A “thiol protecting group” is well understood in the art. A thiol protecting group is a group which prevents the reactivity of the mercapto group during a reaction which modifies some other portion of the molecule and can be easily removed to generate the desired mercapto group. Thiol protecting groups can be found at least in Greene and Wuts, 1999, which is incorporated herein by reference. Some non-limiting examples of thiol protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; acyloxy groups such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like.

As used herein, the term “patient” or “subject” refers to a living mammalian organism, such as a human, monkey, cow, horse, sheep, goat, dog, cat, mouse, rat, guinea pig, or transgenic species thereof. In certain embodiments, the patient or subject is a primate. Non-limiting examples of human subjects are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

The term “pharmaceutically acceptable carrier,” as used herein means a pharmaceutically-acceptable material, composition or vehicle, such as a liquid or solid filler, diluent, excipient, solvent or encapsulating material, involved in carrying or transporting a chemical agent.

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an inhibitor according to the present invention. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≦15%, more preferably ≦10%, even more preferably ≦5%, or most preferably ≦1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### II. COMPOUNDS AND SYNTHETIC METHODS OF THE PRESENT DISCLOSURE THEREOF

In the present invention, synthetic methods to prepare novel analogs of vinaxanthone and xanthofulvin are described. The novel analogs of vinaxanthone and xanthofulvin described in this disclosure can be prepared according to the methods described in the Examples section below. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

The novel analogs of vinaxanthone and xanthofulvin described in this disclosure may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. The analogs of vinaxanthone and xanthofulvin may occur as racemates and racemic mixtures, single enantiomers, diastereomeric mixtures and individual diastereomers. In some embodiments, a single diastereomer is obtained. The chiral centers of the present invention can have the S or the R configuration.

In addition, atoms making up the analogs of vinaxanthone and xanthofulvin of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present invention may be replaced by a silicon atom(s). Furthermore, it is contemplated that one or more oxygen atom(s) of the novel analogs of vinaxanthone and xanthofulvin may be replaced by a sulfur or selenium atom(s).

The novel analogs of vinaxanthone and xanthofulvin may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical advantages over, compounds known in the prior art for use in the indications stated herein.

Compounds of the present invention may also exist in prodrug form. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs.

Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

### III. G-COUPLED PROTEIN RECEPTORS

G-protein-coupled receptors (GPCRs) make up more than 2% of the total genes in the human genome and the vast majority of cell surface proteins. To date, 50-60% of all current therapeutics target GPCRs. The GPCR Succinate Receptor (GPR91) has been linked to a variety of human diseases including spinal cord injury, local inflammatory, hypertension, and retinopathy. Succinate is most known as an intermediate in the Krebs cycle. However, studies have shown that upon injury, release of succinate causes inflammation and vascular proliferation.

Given the ubiquitous nature of G-coupled protein receptors in disease pathologies, the compositions of the present disclosure, which modulate G-coupled protein receptor activity, can be used to treat diseases associated with SUCNR1 including cardiovascular disorders, dermatological disorders, metabolic diseases, cancer disorders, gastrointestinal and liver diseases, hematological disorders, reproductive disorders, endocrinal diseases, inflammatory diseases, muscle-skeleton disorders, neurological disorders, urological disorders, respiratory diseases and ophthalmological diseases.

### IV. NEURAL REGENERATION AND SPINAL CORD INJURIES

Nerve cells have no mitotic potential in an adult and thus, once they are damaged, the damage can persist over a long period of time. Without being bound by theory, the lack of no regeneration potential especially in the central nervous system, i.e., the brain and spinal cord. Lack of the regeneration potential in the central nerves can be regarded as one of the reasons that there have been no established therapies for traumatic injuries such as spinal cord injury, nor for neurodegenerative diseases such as Alzheimer's disease and Parkinson's disease. On the other hand, peripheral nerves possess regeneration potential. Their axons can regenerate and their functions can be recovered even after having been severed. However, even the peripheral nerves having regeneration potential are entirely unable to outgrow in the brain or spinal cord leading to the fact that some substances in the central nervous system inhibits nerve outgrowth. Semaphorin was first isolated as a factor involved in nervous system formation and the protein has been identified as a factor which collapses nerve growth cone and suppresses axon outgrowth. Semaphorin 3A is the most studied and is known to induce growth cone collapse of the cultured nerve cells at as low as 10 pM concentration in a short period of time.

Semaphorins, which are short range inhibitory proteins that act as axonal growth cone guidance molecules, are synthesized by neurons during axon pathfinding. Repulsive guidance cue semaphorin-3A is a gene of the semaphorin family and is expressed by motorneurons to control motor axonal pathfinding. Axon pathfinding is the process by which neurons follow very precise paths, sends out axons, and react to specific chemical environments to reach the correct endpoint. During nervous system development, guidance cues, such as Semaphorin-3A induce the collapse and paralysis of neuronal growth cones. Semaphorins are endogenous proteins which are identified as a factor that can retract the nerve growth cone and suppress the axonal growth, e.g., semaphorin 3A. Certain groups of xanthone compounds have been shown to inhibiting the action of semaphorin 3A (i.e., semaphorin inhibitor) and the action for promoting neuroregeneration. As used herein the term “Sema3A” denotes Semaphorin-3A which is a secreted protein, or chemorepulser, secreted by surrounding tissues to guide migrating cells and axons in the developing nervous system of an organism which is critical for the precise formation of neurons and vasculature.

The growth cone collapse activity of semaphorin means an activity to make growth cones disappear. The compounds of the present disclosure may be used to promote action on central and/or peripheral nerve regeneration. In other embodiments, the present disclosure provides a compound having suppressing action on the growth cone collapse activity and/or the nerve outgrowth inhibitory activity in a collagen gel. In still other embodiments, the present disclosure provides a compound having suppressing action on both growth cone collapse activity of semaphorin and nerve outgrowth inhibitory activity in a collagen gel. In some embodiments and without being bound by theory, the compounds of the present disclosure exert their neural regenerative capacity by activating SUCNR1 which causes neurons to secrete growth factors including VEGF and angiopoietins. This GPCR has been previously characterized as a key response element to cellular stress and activation of SUCNR1 by succinate leads to the production of growth factors that in turn promote angiogenesis, neuronal growth, and cellular survival. Small molecule-mediated allosteric modulation of GPCRs represents a promising approach for drug development, in particular for CNS disorders.

The natural product vinaxanthone was first isolated by Yokose in 1991 from a broth of Penicillium vinaceum and subsequently in 2003 by Kumagai from Penicillium sp. SPF-3059. The isolation by Kumagai was guided by the ability of vinaxanthone to act as an inhibitor of semaphorin 3A (Sema3A), an extracellular matrix protein that contributes to the inhibition of axonal regeneration. Without being bound by theory, the protein Sema3A suppresses axonal regeneration by acting on microtubules and the actin cyctoskeleton causing growth cone collapse, preventing the extension of fledgling axons following injury. In some embodiments, vinaxanthone and analogs thereof possesses Sema3A inhibitory activity with an IC₅₀ value of 0.1 μg/mL with no observable cytotoxicity effects at concentrations >1000 times the effective dose.

### V. INFECTIONS

Hyper stimulation of the immune system promotes inflammation, autoimmunity and transplant rejection. In some embodiments, the present disclosure provides antagonists of SUCNR1 may reverse this pathophysiology. Without being bound by theory, SUCNR1 can also activates dendritic cells (DCs). Upon stimulation, DCs detect pathogens and injured tissue and are involved in the immune system's innate immunity.

A. Bacterial Infections

In some aspects of the present disclosure, the compounds disclosed herein may be used to treat a bacterial infection. While humans contain numerous different bacteria on and inside their bodies, an imbalance in bacterial levels or the introduction of pathogenic bacteria can cause a symptomatic bacterial infection. Pathogenic bacteria cause a variety of different diseases including but not limited to numerous foodborne illness, typhoid fever, tuberculosis, pneumonia, syphilis, and leprosy.

Additionally, different bacteria have a wide range of interactions with body and those interactions can modulate ability of the bacteria to cause an infection. For example, bacteria can be conditionally pathogenic such that they only cause an infection under specific conditions. For example, Staphylococcus and Streptococcus bacteria exist in the normal human bacterial biome, but these bacteria when they are allowed to colonize other parts of the body causing a skin infection, pneumonia, or sepsis. Other bacteria are known as opportunistic pathogens and only cause diseases in a patient with a weakened immune system or another disease or disorder.

Bacteria can also be intracellular pathogens which can grow and reproduce within the cells of the host organism. Such bacteria can be divided into two major categories as either obligate intracellular parasites or facultative intracellular parasites. Obligate intracellular parasites require the host cell in order to reproduce and include such bacteria as but are not limited to Chlamydophila, Rickettsia, and Ehrlichia which are known to cause pneumonia, urinary tract infections, typhus, and Rocky Mountain spotted fever. Facultative intracellular parasites can reproduce either intracellular or extracellular. Some non-limiting examples of facultative intracellular parasites include Salmonella, Listeria, Legionella, Mycobacterium, and Brucella which are known to cause food poisoning, typhoid fever, sepsis, meningitis, Legionnaire's disease, tuberculosis, leprosy, and brucellosis.

Finally, bacterial infections could be targeted to a specific location in or on the body. For example, bacteria could be harmless if only exposed to the specific organs, but when it comes in contact with a specific organ or tissue, the bacteria can begin replicating and cause a bacterial infection.

i. Gram Positive Bacteria

In some aspects of the present disclosure, the compounds disclosed herein may be used to treat a bacterial infection by a gram positive bacteria. Gram positive bacteria contain a thick peptidoglycan layer within the cell wall which prevents the bacteria from releasing the stain when dyed with crystal violet. Without being bound by theory, the gram positive bacteria are often more susceptible to antibiotics. Generally, gram positive bacteria, in addition to the thick peptidoglycan layer, also comprise a lipid monolayer and contain teichoic acids which react with lipids to form lipoteichoic acids that can act as a chelating agent. Additionally, in gram positive bacteria, the peptidoglycan layer is outer surface of the bacteria. Many gram positive bacteria have been known to cause disease including, but are not limited to, Streptococcus, Straphylococcus, Corynebacterium, Enterococcus, Listeria, Bacillus, Clostridium, Rathybacter, Leifsonia, and Clavibacter.

ii. Gram Negative Bacteria

In some aspects of the present disclosure, the compounds disclosed herein may be used to treat a bacterial infection by a gram negative bacteria. Gram negative bacteria do not retain the crystal violet stain after washing with alcohol. Gram negative bacteria, on the other hand, have a thin peptidoglycan layer with an outer membrane of lipopolysaccharides and phospholipids as well as a space between the peptidoglycan and the outer cell membrane called the periplasmic space. Gram negative bacterial generally do not have teichoic acids or lipoteichoic acids in their outer coating. Generally, gram negative bacteria also release some endotoxin and contain prions which act as molecular transport units for specific compounds. Most bacteria are gram negative. Some non-limiting examples of gram negative bacteria include Bordetella, Borrelia, Burcelia, Campylobacteria, Escherichia, Francisella, Haemophilus, Helicobacter, Legionella, Leptospira, Neisseria, Pseudomonas, Rickettsia, Salmonella, Shigella, Treponema, Vibrio, and Yersinia.

iii. Gram Indeterminate Bacteria

In some aspects of the present disclosure, the compounds disclosed herein may be used to treat a bacterial infection by a gram indeterminate bacteria. Gram indeterminate bacteria do not full stain or partially stain when exposed to crystal violet. Without being bound by theory, a gram indeterminate bacteria may exhibit some of the properties of the gram positive and gram negative bacteria. A non-limiting example of a gram indeterminate bacteria include mycobacterium tuberculosis or mycobacterium leprae.

B. Viral Infections

In some aspects of the present disclosure, the compounds disclosed herein may be used to treat a viral infection. Similarly, virus can also exist in pathogenic form which can lead to human diseases. Viral infections are typically not treated directly but rather symptomatically since virus often have a self-limiting life cycle. Viral infections can also be more difficult to diagnosis than a bacterial infection since viral infections often do result in the concombinent increase in white blood cell counts. Some non-limiting examples of pathogenic virus include influenza virus, smallpox, BK virus, JC virus, human papillomavirus, adenovirus, herpes simplex type 1, herpes simplex type 2, varicella-zoster virus, Epstein barr virus, human cytomegalovirus, human herpesvirus type 8, Norwalk virus, human bocavirus, rubella virus, hepatitis E virus, hepatitis B virus, human immunodeficiency virus (HIV), Ebola virus, rabies virus, rotavirus, and hepatitis D virus.

### VI. RETINOPATHY

Release of succinate (and subsequent binding to SUCNR1) causes inflammation and vascular proliferation. In some embodiments, a compound such as a compound of the present disclosure which can modulate the activity of SUCNR1 may be used to treat retinopathy. In some embodiments, the retinopathy is diabetic retinopathy. For diabetics, excessive angiogenesis of the retina and cornea leads to retinopathy, progressing to blindness, if left untreated. In some embodiments, the compounds of the present disclosure can be used to treat excessive angiogenesis of the retina and/or cornea. In some embodiments, the compounds of the present disclosure may be used to treat or prevent retinopathy.

### VII. HYPERPROLIFERATIVE DISEASES

While hyperproliferative diseases can be associated with any disease which causes a cell to begin to reproduce uncontrollably, the prototypical example is cancer. One of the key elements of cancer is that the cell's normal apoptotic cycle is interrupted and the cells divide uncontrollably. In some embodiments, the increased cellular division requires additional resources be provided to the cancer cells. The increased resources results in the growth of additional vasculature to the tumor to provide more blood flow and thus more nutrients and oxygen. In some aspects, the compounds of the present disclosure may be used to decrease the vasculature development of a tumor. In various aspects, it is anticipated that the compounds of the present disclosure may be used to treat virtually any malignancy.

Cancer cells that may be treated with the compounds according to the embodiments include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### VIII. PHARMACEUTICAL FORMULATIONS AND ROUTES OF ADMINISTRATION

For administration to a mammal in need of such treatment, the analogs of vinaxanthone and xanthofulvin in a therapeutically effective amount are ordinarily combined with one or more excipients appropriate to the indicated route of administration. The analogs of vinaxanthone and xanthofulvin may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol, and tableted or encapsulated for convenient administration. Alternatively, the analogs of vinaxanthone and xanthofulvin may be dissolved in water, polyethylene glycol, propylene glycol, ethanol, corn oil, cottonseed oil, peanut oil, sesame oil, benzyl alcohol, sodium chloride, and/or various buffers. Other excipients and modes of administration are well and widely known in the pharmaceutical art.

The pharmaceutical compositions useful in the present invention may be subjected to conventional pharmaceutical operations such as sterilization and/or may contain conventional pharmaceutical carriers and excipients such as preservatives, stabilizers, wetting agents, emulsifiers, buffers, etc.

The analogs of vinaxanthone and xanthofulvin may be used by administering the compound through a variety of methods, e.g., orally or by injection (e.g. subcutaneous, intravenous, intraperitoneal, etc.). Depending on the route of administration, the novel analogs of vinaxanthone and xanthofulvin may be coated in a material to protect the compound from the action of acids and other natural conditions which may inactivate the compound. They may also be administered by continuous perfusion/infusion of a disease or wound site.

To administer the therapeutic compound by other than parenteral administration, it may be necessary to coat the analogs of vinaxanthone and xanthofulvin with, or co-administer the novel analogs of vinaxanthone and xanthofulvin with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The analogs of vinaxanthone and xanthofulvin may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion are also envisioned. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the analogs of vinaxanthone and xanthofulvin in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The analogs of vinaxanthone and xanthofulvin can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the analogs of vinaxanthone and xanthofulvin may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the analogs of vinaxanthone and xanthofulvin in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of the novel analogs of vinaxanthone and xanthofulvin calculated to produce the desired therapeutic effect in association with the required pharmaceutical carrier. The specification for the dosage unit forms of the invention are dictated by and directly dependent on (a) the unique characteristics of the analogs of vinaxanthone and xanthofulvin described in this invention and the particular therapeutic effect to be achieved, and (b) the limitations inherent in the art of compounding such a therapeutic compound for the treatment of a selected condition in a patient.

The therapeutic compound may also be administered topically to the skin, eye, or mucosa. Alternatively, if local delivery to the lungs is desired the therapeutic compound may be administered by inhalation in a dry-powder or aerosol formulation. Furthermore, the analogs of vinaxanthone and xanthofulvin can be administered locally to the spinal cord or the central nervous system to encourage neural regeneration.

In some embodiments, the compounds of the present invention are administered using a hydrogel or other polymers for continuous drug delivery. In some aspects, the hydrogel or polymer for continuous drug delivery include but are not limited to those described by Madigan, et al., 2009; Baier Leach, 2003; Struve, et al., 2005; and Gros, et al., 2010.

The analogs of vinaxanthone and xanthofulvin describe in this disclosure are administered at a therapeutically effective dosage sufficient to treat a condition associated with a condition in a patient. For example, the efficacy of the analogs of vinaxanthone and xanthofulvin can be evaluated in an animal model system that may be predictive of efficacy in treating the disease in humans, such as the model systems shown in the examples and drawings.

The actual dosage amount of the analogs of vinaxanthone and xanthofulvin of the present disclosure or composition comprising the inhibitors of the present disclosure administered to a subject may be determined by physical and physiological factors such as age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

An effective amount typically will vary from about 1 mg/kg to about 50 mg/kg, in one or more dose administrations daily, for one or several days (depending of course of the mode of administration and the factors discussed above). In some particular embodiments, the amount is less than 5,000 mg per day with a range of 10 mg to 4500 mg per day.

The effective amount may be less than 10 mg/kg/day, less than 50 mg/kg/day, less than 100 mg/kg/day, less than 250 mg/kg/day. It may alternatively be in the range of 1 mg/kg/day to 250 mg/kg/day.

In other non-limiting examples, a dose may also comprise from about 0.1 mg/kg/body weight, about 1 mg/kg/body weight, about 10 g/kg/body weight, about 50 g/kg/body weight, or more per administration, and any range derivable therein. In non-limiting examples of a derivable range from the numbers listed herein, a range of about 1 mg/kg/body weight to about 50 mg/kg/body weight, about 5 g/kg/body weight to about 10 g/kg/body weight, etc., can be administered, based on the numbers described above.

In certain embodiments, a pharmaceutical composition of the present disclosure may comprise, for example, at least about 0.1% of an inhibitor described in the present disclosure. In other embodiments, the compound of the present disclosure may comprise between about 0.25% to about 75% of the weight of the unit, or between about 25% to about 60%, or between about 1% to about 10%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The analogs of vinaxanthone and xanthofulvin may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may taken orally and that the timing of which is or is not dependent upon food intake. Thus, for example, the agent can be taken every morning and/or every evening, regardless of when the subject has eaten or will eat.

### IX. COMBINATION THERAPY

In addition to being used as a monotherapy, the analogs of vinaxanthone and xanthofulvin described in the present invention may also find use in combination therapies. Effective combination therapy may be achieved with a single composition or pharmacological formulation that includes both agents, or with two distinct compositions or formulations, administered at the same time, wherein one composition includes an analogs of vinaxanthone and xanthofulvin, and the other includes the second agent(s). The other therapeutic modality may be administered before, concurrently with, or following administration of the analogs of vinaxanthone and xanthofulvin. The therapy using the analogs of vinaxanthone and xanthofulvin may precede or follow administration of the other agent(s) by intervals ranging from minutes to weeks. In embodiments where the other agent and the compounds of the present disclosure which act as the analogs of vinaxanthone and xanthofulvin are administered separately, one would generally ensure that a significant period of time did not expire between the time of each delivery, such that each agent would still be able to exert an advantageously combined effect. In such instances, it is contemplated that one would typically administer the analogs of vinaxanthone and xanthofulvin and the other therapeutic agent within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of a novel analogs of vinaxanthone and xanthofulvin, or the other agent will be desired. In this regard, various combinations may be employed. By way of illustration, where the novel analogs of vinaxanthone and xanthofulvin is “A” and the other agent is “B”, the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A
B/A/A/B B/B/B/A
A/A/A/B B/A/A/A A/B/A/A A/A/B/A A/B/B/B
B/A/B/B B/B/A/B

Other combinations are likewise contemplated. Non-limiting examples of pharmacological agents that may be used in the present invention include any pharmacological agent known to be of benefit in the treatment of a cancer or hyperproliferative disorder or disease. In some embodiments, combinations of the analogs of vinaxanthone and xanthofulvin with a cancer targeting immunotherapy, radiotherapy, chemotherapy, or surgery are contemplated. Also contemplated is a combination of the analogs of vinaxanthone and xanthofulvin with more than one of the above mentioned methods including more than one type of a specific therapy.

1. Agents which Promote Neural Regeneration

In some embodiments of the present disclosure, the compounds can be used in conjugation with one or more additional agents that promote neural regeneration. Some non-limiting examples of types of agents which may be used with the present invention include gene therapy, biologics, a small molecular neural regeneration promoter, or stem-cell based approach. Such agents are taught by Wilson and Danishefsky, 2006, which is incorporated herein by reference.

2. Antibiotics

The term “antibiotics” are drugs which may be used to treat a bacterial infection through either inhibiting the growth of bacteria or killing bacteria. In some embodiments of the present disclosure, the compounds may be used in conjunction with one or more antibiotics. Without being bound by theory, it is believed that antibiotics can be classified into two major classes: bactericidal agents that kill bacteria or bacteriostatic agents that slow down or prevent the growth of bacteria.

The first commercially available antibiotic was released in the 1930's. Since then, many different antibiotics have been developed and widely prescribed. In 2010, on average, 4 in 5 Americans are prescribed antibiotics annually. Given the prevalence of antibiotics, bacteria have started to develop resistance to specific antibiotics and antibiotic mechanisms. Without being bound by theory, the use of antibiotics in combination with another antibiotic may modulate resistance and enhance the efficacy of one or both agents.

In some embodiments, antibiotics can fall into a wide range of classes. In some embodiments, the compounds of the present disclosure may be used in conjunction with another antibiotic. In some embodiments, the compounds may be used in conjunction with a narrow spectrum antibiotic which targets a specific bacteria type. In some non-limiting examples of bactericidal antibiotics include penicillin, cephalosporin, polymyxin, rifamycin, lipiarmycin, quinolones, and sulfonamides. In some non-limiting examples of bacteriostatic antibiotics include macrolides, lincosamides, or tetracyclines. In some embodiments, the antibiotic is an aminoglycoside such as kanamycin and streptomycin, an ansamycin such as rifaximin and geldanamycin, a carbacephem such as loracarbef, a carbapenem such as ertapenem, imipenem, a cephalosporin such as cephalexin, cefixime, cefepime, and ceftobiprole, a glycopeptide such as vancomycin or teicoplanin, a lincosamide such as lincomycin and clindamycin, a lipopeptide such as daptomycin, a macrolide such as clarithromycin, spiramycin, azithromycin, and telithromycin, a monobactam such as aztreonam, a nitrofuran such as furazolidone and nitrofurantoin, an oxazolidonones such as linezolid, a penicillin such as amoxicillin, azlocillin, flucloxacillin, and penicillin G, an antibiotic polypeptide such as bacitracin, polymyxin B, and colistin, a quinolone such as ciprofloxacin, levofloxacin, and gatifloxacin, a sulfonamide such as silver sulfadiazine, mefenide, sulfadimethoxine, or sulfasalazine, or a tetracycline such as demeclocycline, doxycycline, minocycline, oxytetracycline, or tetracycline. In some embodiments, the compounds could be combined with a drug which acts against mycobacteria such as cycloserine, capreomycin, ethionamide, rifampicin, rifabutin, rifapentine, and streptomycin. Other antibiotics that are contemplated for combination therapies may include arsphenamine, chloramphenicol, fosfomycin, fusidic acid, metronidazole, mupirocin, platensimycin, quinupristin, dalfopristin, thiamphenicol, tigecycline, tinidazole, or trimethoprim.

3. Antivirals

The term “antiviral” or “antiviral agents” are drugs which may be used to treat a viral infection. In general, antiviral agents act via two major mechanisms: preventing viral entry into the cell and inhibiting viral synthesis. In some embodiments of the present disclosure, the compounds may be used in conjunction with one or more antiviral agents. Without being bound by theory, viral replication can be inhibited by using agents that mimic either the virus-associated proteins and thus block the cellular receptors or using agents that mimic the cellular receptors and thus block the virus-associated proteins. Furthermore, agents which cause an uncoating of the virus can also be used as antiviral agents.

The second mechanism of viral inhibition is preventing or interrupting viral synthesis. Such drugs can target different proteins associated with the replication of viral DNA including reverse transcriptase, integrase, transcription factors, or ribozymes. Additionally, the therapeutic agent interrupts translation by acting as an antisense DNA strain, inhibiting the formation of protein processing or assembly, or acting as virus protease inhibitors. Finally, an anti-viral agent could additionally inhibit the release of the virus after viral production in the cell.

Additionally, anti-viral agents could modulate the bodies own immune system to fight a viral infection. Without being bound by theory, the anti-viral agent which stimulates the immune system may be used with a wide variety of viral infections.

In some embodiments, the present disclosure provides methods of using the disclosed compounds in a combination therapy with an anti-viral agent as described above. In some non-limiting examples, the anti-viral agent is abacavir, aciclovir, acyclovir, adefovir, amantadine, amprenavir, ampligen, arbidol, atazanavir, atripla, balavir, boceprevirertet, cidofovir, combivir, dolutegravir, daruavir, delavirdine, didanosine, docosanol, edoxudine, efavirenz, emtricitabine, enfuvirtide, entecavir, ecoliever, famciclovir, fomivirsen, fosamprenavir, foscarnet, fosfonet, ganciclovir, ibacitabine, imunovir, idoxuridine, imiquimod, indinavir, inosine, interferon type I, type II, and type III, lamivudine, lopinavir, loviride, maraviroc, moroxydine, methisazone, nelfinavir, nevirapine, nexavir, oseltamivir, penciclovir, peramivir, pleconaril, podophyllotoxin, raltegravir, ribavirin, rimantadine, ritonavir, pyramidine, saquinavir, sofosbuvir, stavudine, telaprevir, tenofovir, tenofovir disoproxil, tipranavir, trifluridine, trizivir, tromantadine, truvada, traporved, valaciclovir, valganciclovir, vicriviroc, vidarabine, viramidine, zalcitabine, zanamivir, or zidovudine. In some embodiments, the anti-viral agents is an anti-retroviral, a fusion inhibitor, an integrase inhibitor, an interferon, a nucleoside analogues, a protease inhibitor, a reverse transcriptase inhibitor, a synergistic enhancer, or a natural product such as tea tree oil.

4. Chemotherapy

The term “chemotherapy” refers to the use of drugs to treat cancer. A “chemotherapeutic agent” is used to connote a compound or composition that is administered in the treatment of cancer. In some embodiments of the present disclosure, the compounds may be used in conjunction with one or more chemotherapeutic agents These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

5. Radiotherapy

In some embodiments of the present disclosure, the compounds may be used in conjunction with radiotherapy. Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

6. Immunotherapy

In the context of cancer treatment, immunotherapeutics, generally, rely on the use of immune effector cells and molecules to target and destroy cancer cells. In some embodiments of the present disclosure, the compounds may be used in conjunction with one or more immunotherapy. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance antitumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

7. Surgery

Approximately 60% of persons with cancer will undergo surgery of some type, which includes preventative, diagnostic or staging, curative, and palliative surgery. In some embodiments of the present disclosure, the compounds may be used in conjunction with surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some particular embodiments, after removal of the tumor, an adjuvant treatment with a compound of the present disclosure is believe to be particularly efficacious in reducing the reoccurance of the tumor. Additionally, the compounds of the present disclosure can also be used in a neoadjuvant setting.

It also should be pointed out that any of the foregoing therapies may prove useful by themselves in treating cancer.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Methods and Materials

All reactions were performed in flame dried round bottom or modified Schlenk (Kjedahl shape) flasks fitted with rubber septa under a positive pressure of argon, unless otherwise indicated. Air- and moisture-sensitive liquids and solutions were transferred via syringe or canula. Organic solutions were concentrated by rotary evaporation at 20 torr. Methylene chloride (CH₂Cl₂) and tetrahydrofuran (THF) were purified using a Pure-Solv MD-5 Solvent Purification System (Innovative Technology). Acetonitrile (MeCN) was purified using a Vac 103991 Solvent Purification System (Vacuum Atmospheres). Dimethoxyethane (DME) was purchased from Acros (99+%, stabilized with BHT), methanol (MeOH) was purchased from Sigma-Aldrich (99.8%, anhydrous), ethanol (EtOH) was purchased from Pharmco-Aaper (200 proof, absolute). All other reagents were used directly from the supplier without further purification unless noted. Analytical thin-layer chromatography (TLC) was carried out using 0.2 mm commercial silica gel plates (silica gel 60, F254, EMD chemical) and visualized using a UV lamp and/or aqueous ceric ammonium molybdate (CAM) or aqueous potassium permanganate (KMnO₄) stain. Infrared spectra were recorded on a Nicolet 380 FTIR using neat thin film or KBr pellet technique. High-resolution mass spectra (HRMS) were recorded on a Karatos MS9 and are reported as m/z (relative intensity). Accurate masses are reported for the molecular ion [M+Na]⁺, [M+H], [M⁺], or [M−H]. Nuclear magnetic resonance spectra (¹H NMR and ¹³C NMR) were recorded with a Varian Gemini [(400 MHz, ¹H at 400 MHz, ¹³C at 100 MHz), (500 MHz, ¹³C at 125 MHz), (600 MHz, ¹³C at 150 MHz)]. For CDCl₃ solutions the chemical shifts are reported as parts per million (ppm) referenced to residual hydrogen or carbon of the solvent; CHCl₃ δ H (7.26 ppm) and CDCl₃ δ C (77.0 ppm). For (CD₃)₂SO solutions the chemical shifts are reported as parts per million (ppm) referenced to residual hydrogen or carbon of the solvents; (CD₃)(CHD₂)SO δ H (2.50 ppm) or (CD₃)₂SO δ C (39.5 ppm). Coupling constants are reported in Hertz (Hz). Data for ¹H NMR spectra are reported as follows: chemical shift (ppm, referenced to protium; s=singlet, d=doublet, t=triplet, q=quartet, dd=doublet of doublets, td=triplet of doublets, ddd=doublet of doublet of doublets, m=multiplet, coupling constant (Hz), and integration). Melting points were measured on a MEL-TEMP device without corrections.

### Example 2

### Synthesis

The synthesis of vinaxanthone can be carried out through an aldol condensation of an ynone (19) and reactive aldehyde (20) (Scheme 2).

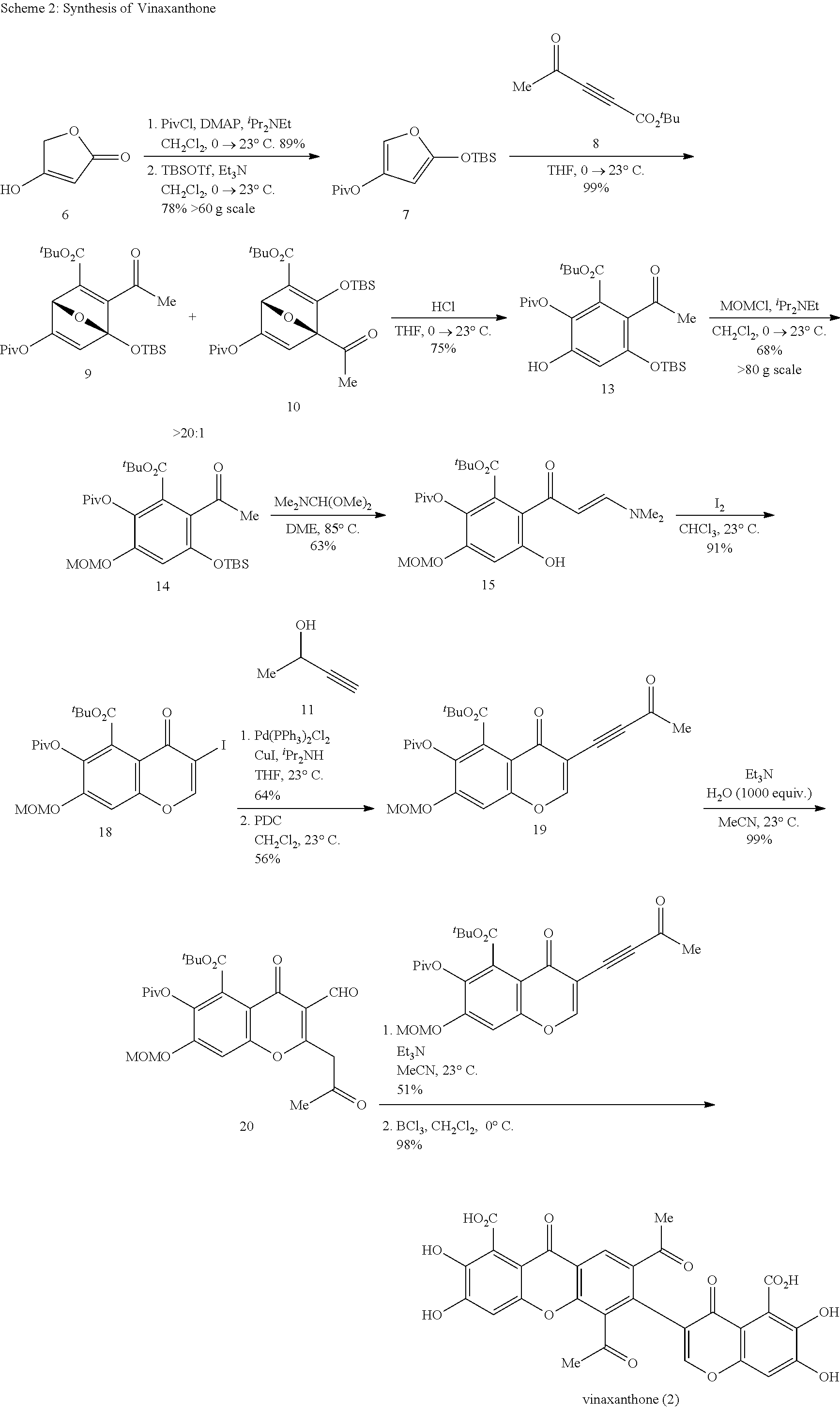

In the presence of wet acetonitrile and triethylamine, the ynone intermediate (19) used in the xanthofulvin synthesis dimerizes to form protected vinaxanthone. The proposed mechanism for the dimerization shown in Scheme 3.

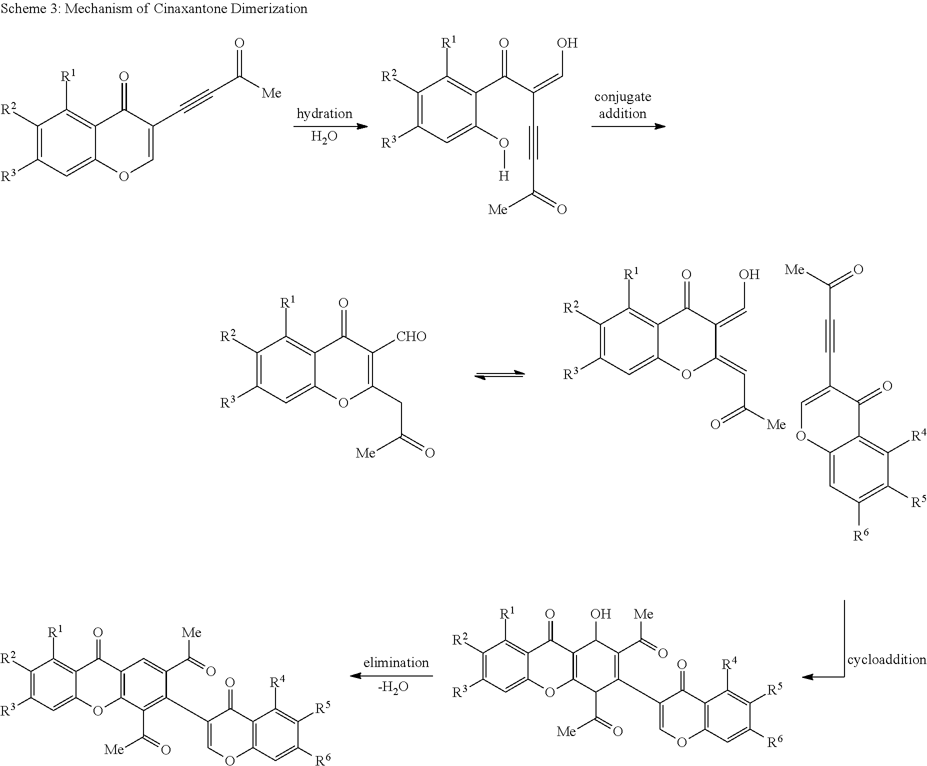

In order to produce the ynone intermediate, the enaminone species (15) with iodine in chloroform to generate the iodochromone species (18). The iodochromone (18) then undergoes a Sonagashira cross-coupling reaction with 3-butyn-2-ol (11) to generate a propargyl alcohol species. The alcohol is then oxidized to the ynone species (19) with pyridinium dichlorochromate (PDC). Placing the ynone (19) in acetonitrile and water (1000 equivalents of H₂O) with 0.5 eq. of triethylamine at room temperature generated the protected vinaxanthone product at 87% yield through formation of a highly reactive aldehyde species (20). The protected vinaxantone is then deprotected through the use of boron trichloride in dichloromethane at 0° C. to generate vinaxanthone (2).

In order to better understand the dimerization reaction, a study of the reaction conditions was carried out. The water mediated rearrangement of 3-ynone chromone 13 and cycloaddition/dehydration sequence was found to be quite efficient when conducted in a single reaction vessel with substoichiometric equivalents of water (Table 1).

| Entry | Base | H₂O (equiv.) | Solvent | Temp (° C.) | % Yield |
|---|---|---|---|---|---|
| 1 | Et₃N | 0 | MeCN | 23 | 0 |
| 2 | Et₃N | 0.1 | MeCN | 23 | 65 |
| 3 | Et₃N | 0.5 | MeCN | 23 | 87 |
| 4 | Et₃N | 1.0 | MeCN | 23 | 67 |
| 5 | Et₃N | 2.0 | MeCN | 23 | 59 |
| 6 | Et₃N | 3.0 | MeCN | 23 | 54 |
| 7 | i-Pr₂NEt | 3.0 | MeCN | 23 | 21 |
| 8 | DABCO | 3.0 | MeCN | 23 | 5 |
| 9 | Et₃N | 3.0 | CH₂Cl₂ | 23 | 9 |
| 10 | Et₃N | 3.0 | dioxane | 23 | 9 |
| 11 | Et₃N | 3.0 | DMF | 23 | 22 |
| 12 | Et₃N | 3.0 | MeCN | 0 | 33 |
| 13 | Et₃N | 3.0 | MeCN | 82 | 21 |

Limiting the amount of water employed demonstrated the reaction can proceed catalytically with respect to water (0.1 equiv.) in 65% yield (entry 2). It is also important to note that if excess water is present, greater than 50% of the 3-ynone chromone undergoes the rearrangement before the cycloaddition reaction occurs. In this scenario the remaining 3-ynone chromone becomes the limiting reagent, resulting in diminished yields.

### Example 3

### Biological Activity of the Compounds

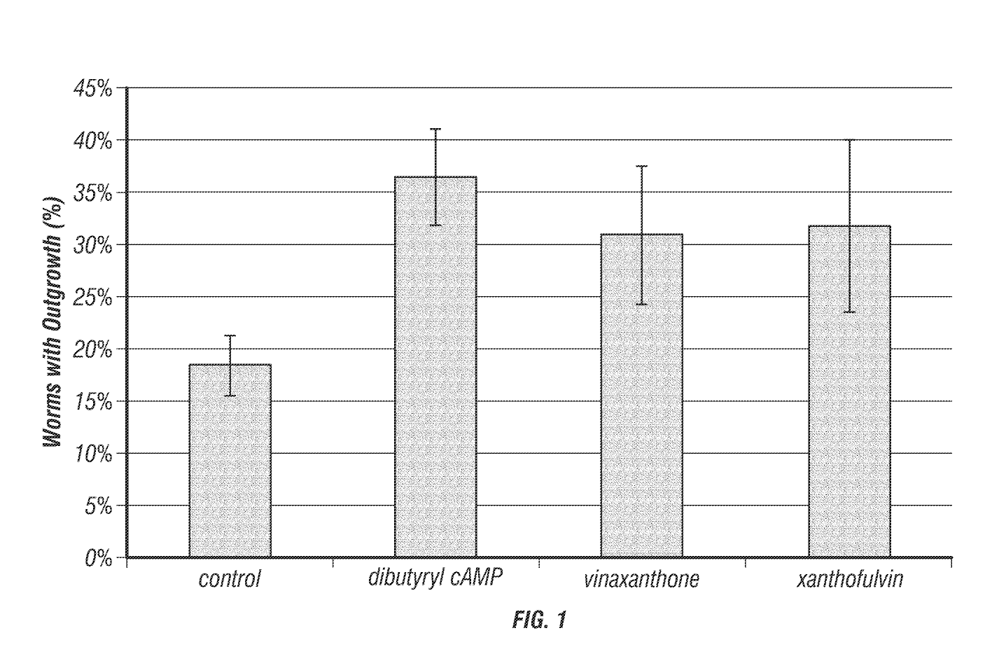

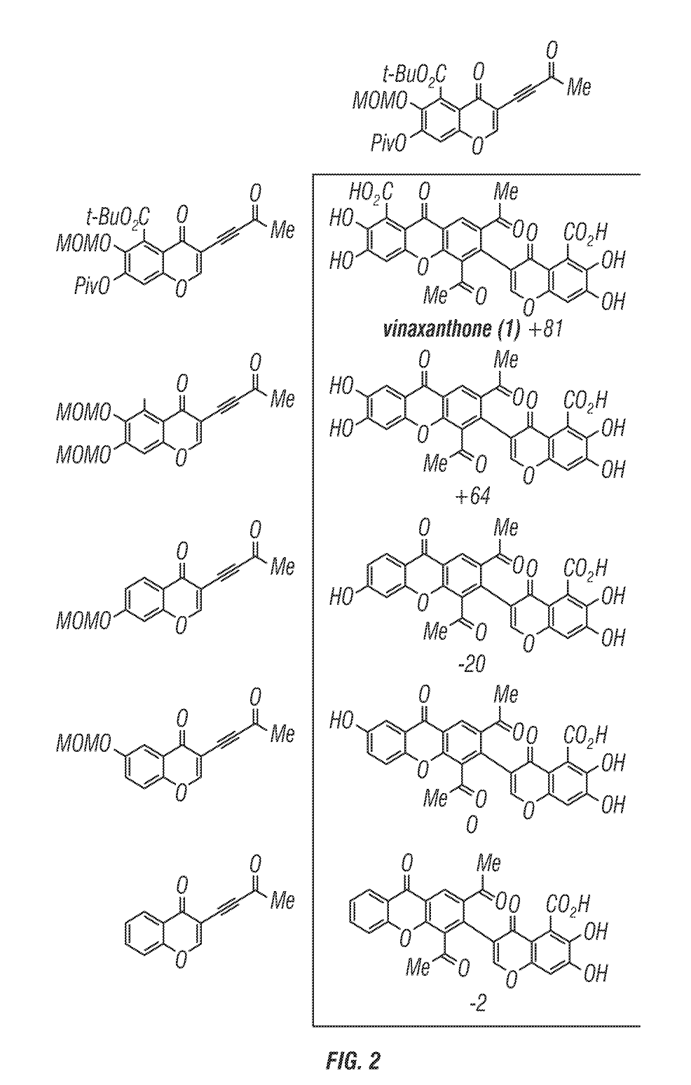

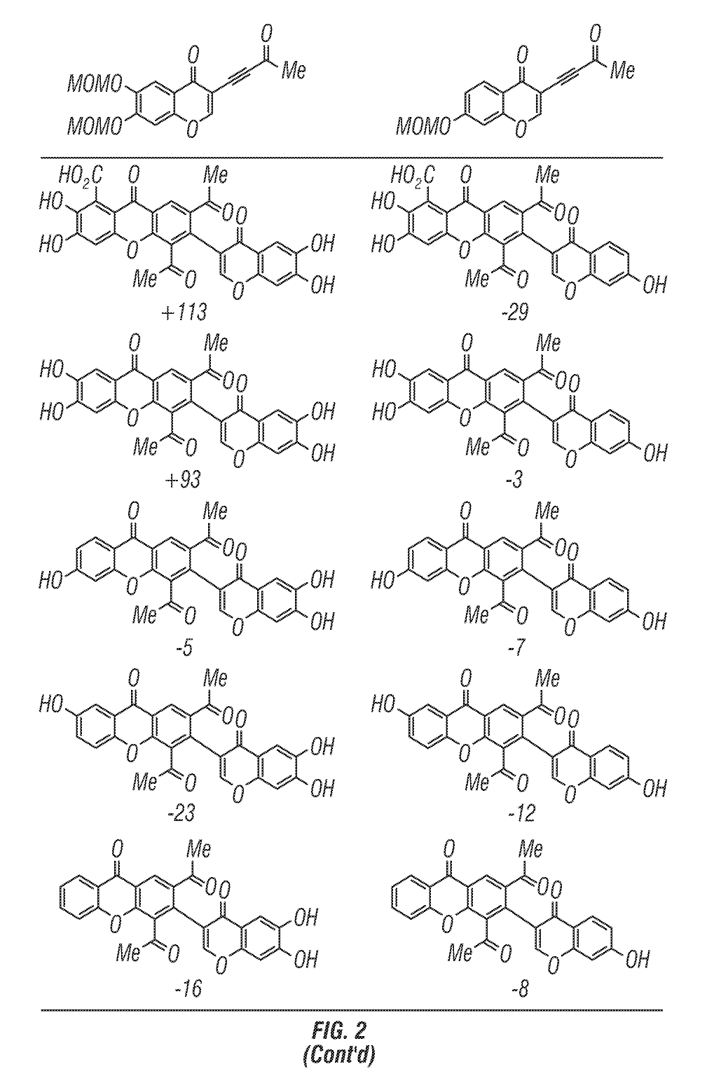

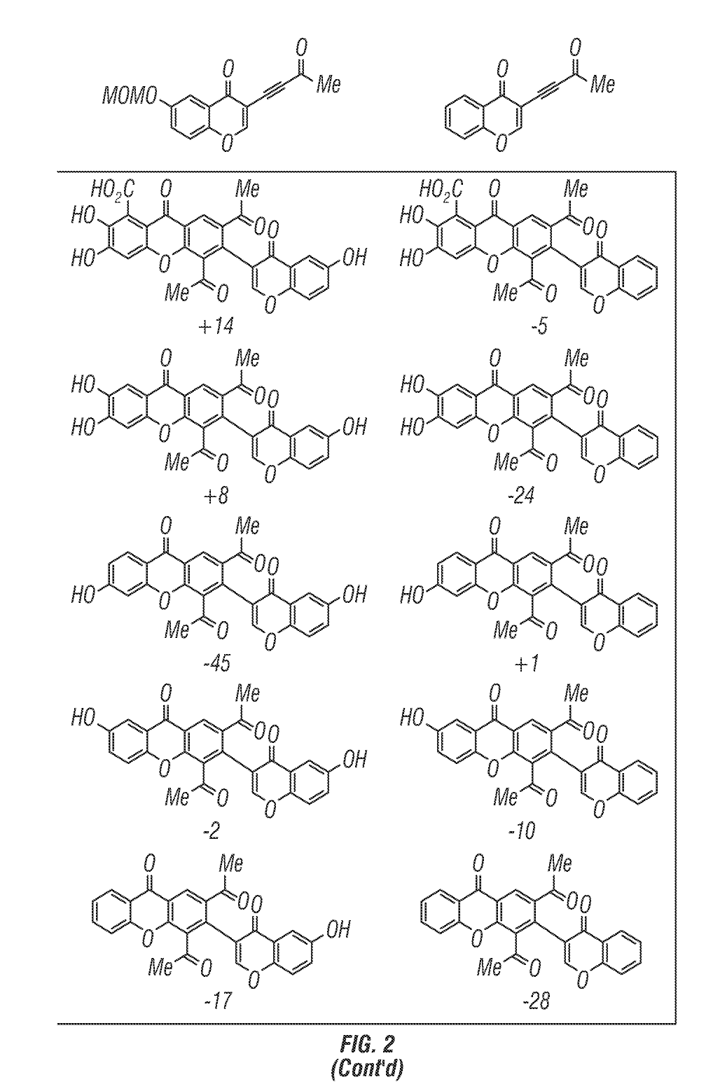

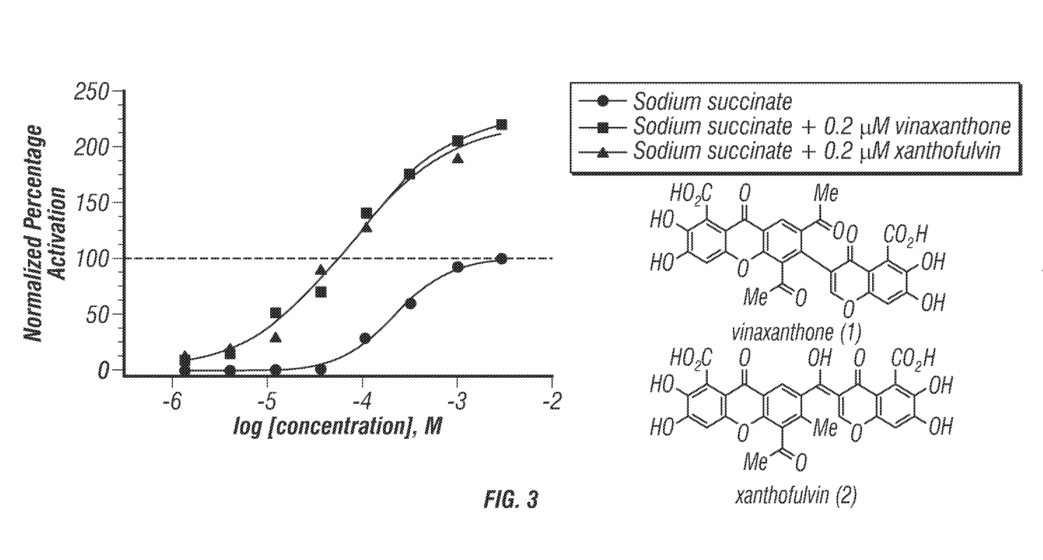

FIG. 1 shows the bar graph of the outgrowth of GFP-labeled cholinergic neurons in vivo in C. elegans after treatment with dibutyryl cAMP, xanthofulvin, and vinaxanthone. Control: 0.2% DMSO in M9 buffer. This activity is comparable to dibutyryl cAMP, which promotes branching in 36% of animals at 2 mm. Additionally, several analogs were prepared and their biological activity was measured. The modulation of the analogs activity on SUCNR1 is shown in FIG. 2. Furthermore, administration of the compounds of the present disclosure was found to particular efficacious when administered with succinate as can be seen in FIG. 3. Dose ratios of 0.33 and 0.32 with efficacy values of 230% and 222% were achieved using vinaxanthone and xanthofulvin, respectively, when compared to sodium succinate alone.

### Example 4

### Characterization of Compounds

A. Conversion and Formation of Starting Material and Intermediates

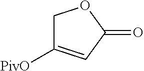

### 5-oxo-2,5-dihydrofuran-3yl pivalate

To a stirred solution of tetronic acid (6) (25.0 g, 250 mmol, 1.0 equiv.), 4-dimethylaminopyridine (1.53 g, 12.5 mmol, 0.05 equiv.) and N,N-diisopropylethylamine (45.8 mL, 262 mmol, 1.05 equiv.) in CH₂Cl₂ (500 mL, 0.5 M) at 0° C. was added neat pivaloyl chloride (25.9 mL, 262 mmol, 1.05 equiv.) dropwise over 40 minutes. Upon complete addition the reaction mixture was allowed to warm to 23° C. After 16 hours, the reaction mixture was concentrated in vacuo to give an amber oil. The oil was dissolved in Et₂O (500 mL) and washed with H₂O (500 mL). The aqueous layer was extracted with Et₂O (5×500 mL) and the combined organic layers were dried over MgSO₄ and concentrated in vacuo to give tetronate 6 (41.0 g, 223 mmol, 89%) as clear amber crystals (m.p. 46-47° C.).

R_{f}=0.60 (silica gel, 1:1 hexanes:EtOAc); ¹H NMR (400 MHz, CDCl₃): δ 6.00 (t, J=1.4 Hz, 1H), 4.91 (d, J=1.4 Hz, 2H), 1.32 (s, 9H); ¹³C NMR (100 MHz, CDCl₃): δ 173.2, 172.2, 169.1, 100.2, 68.2, 38.3, 26.4; IR (film, υ cm⁻¹): 1779, 1746, 1072.

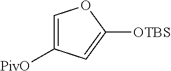

### 5-((tert-butyldimethylsilyl)oxy)furan-3-yl pivalate (7)

To a stirred solution of tetronate (30.0 g, 163 mmol, 1.0 equiv.) and triethylamine (29.8 mL, 212 mmol, 1.3 equiv.) in CH₂Cl₂ (226 mL, 0.72 M) at 0° C. was added neat tert-butyldimethylsilyl triflate (37.8 mL, 165 mmol, 1.01 equiv.) dropwise over 10 minutes. Upon complete addition the reaction mixture was allowed to warm to 23° C. After 1 hour, the reaction mixture was concentrated in vacuo to give an amber oil. The oil was suspended in pentane (200 mL) and stirred for 1 hour at 23° C. The organic layer was decanted and washed with sat. aq. NaHCO₃ (3×100 mL), H₂O (100 mL) and brine (100 mL). The organic layer was dried over Na₂SO₄ and concentrated in vacuo to give furan 7 (37.9 g, 127 mmol, 78%) as an amber oil.

R_{f}=0.55 (silica gel, 20:1 hexanes:EtOAc); ¹H NMR (300 MHz, CDCl₃): δ 7.10 (d, J=1.2 Hz, 1H), 5.15 (d, J=1.2 Hz, 1H), 1.29 (s, 9H), 0.96 (s, 9H), 0.24 (s, 6H); ¹³C NMR (100 MHz, CDCl₃): δ 175.3, 154.3, 139.4, 120.6, 80.1, 39.0, 27.1, 25.4, 18.0, −4.85; IR (film, υ cm⁻¹): 3202, 3141, 1753, 1627; HRMS (ESI) calc. for C₁₅H₂₇O₄Si [M+H]⁺: 299.20000. obs. 299.20000.

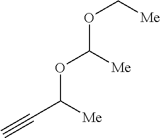

### 3-(1-ethoxyethoxy)but-1-yne

To a stirred solution of 3-butyn-2-ol (11) (100 g, 1.43 mol, 1.0 equiv.) and ethyl vinyl ether (151 mL, 1.57 mol, 1.1 equiv.) in CH₂Cl₂ (3 L, 0.48 M) at 23° C. was added solid pyridinium p-toluenesulfonate (35.9 g, 143 mmol, 0.1 equiv.). After 1 hour, the reaction mixture was diluted with Et₂O (1 L) and washed with brine (2 L). The organic layer was dried over Na₂SO₄ and concentrated in vacuo to give a mixture of diastereomeric alkynes (201 g, 1.41 mol, 99%) as a clear amber oil.

R_{f}=0.40 (silica gel, 1:1 hexanes:EtOAc); ¹H NMR (400 MHz, CDCl₃): δ 4.96 (q, J=5.5 Hz, 1H), 4.85 (q, J=5.5 Hz, 1H), 4.50 (q, J=6.7 Hz, 1H), 4.35 (q, J=6.7 Hz, 1H), 3.75 (m, 1H), 3.62 (m, 1H), 3.53 (m, 2H), 2.40 (s, 1H), 2.39 (s, 1H), 1.46 (d, J=3.1 Hz, 3H), 1.44 (d, J=3.1 Hz, 3H), 1.35 (d, J=2.7 Hz, 3H), 1.34 (d, J=2.7 Hz, 3H), 1.21 (t, J=7.0 Hz, 6H); ¹³C NMR (100 MHz, CDCl₃): δ 98.5, 97.5, 84.5, 83.6, 72.4, 72.0, 61.1, 60.5, 60.0, 59.9, 22.3, 21.9, 20.0, 19.9, 15.2, 14.9; HRMS (EC-CI) calc. for C₈H₁₃O₂ [M+H]⁺: 141.0916. obs. 141.0918.

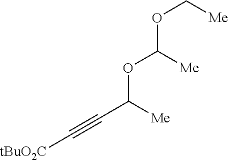 [SMILES: CCOC(C)OC(C)C#CC(=O)OC(C)(C)C]

### tert-butyl 4-(1-ethoxyethoxy)pent-2-ynoate (12)

To a stirred solution of diasteromeric alkynes (110 g, 774 mmol, 1.0 equiv.) in THF (4.5 L, 0.17 M) at −78° C. was added a 2.0 M solution of n-butyllithium in hexanes (404 mL, 808 mmol, 1.05 equiv.). After 15 minutes, neat di-tert-butyl dicarbonate (186 mL, 808 mmol, 1.05 equiv.) was added over 10 minutes. Upon complete addition the reaction mixture was allowed to warm to 23° C. The reaction mixture was diluted with Et₂O (1.5 L) and washed with H₂O (3 L) and brine (3 L). The organic layer was dried over MgSO₄ and concentrated in vacuo to give a mixture of diastereomeric esters (12) (180 g, 743 mmol, 96%) as an amber oil.

R_{f}=0.21 (silica gel, 20:1 hexanes:EtOAc); ¹H NMR (400 MHz, CDCl₃): δ 4.91 (q, J=5.1 Hz, 1H), 4.82 (q, J=5.1 Hz, 1H), 4.56 (q, J=6.8 Hz, 1H), 4.40 (q, J=6.8 Hz, 1H), 3.73 (m, 1H), 3.62 (m, 1H), 3.56 (m, 1H), 3.50 (m, 1H), 1.49 (s, 18H), 1.46 (d, J=1.7 Hz, 6H), 1.34 (d, J=1.4 Hz, 6H) 1.12 (t, J=8.5 Hz, 6H); ¹³C NMR (100 MHz, C₆D₆): δ 152.6, 152.5, 99.3, 98.3, 86.1, 85.2, 82.9, 82.7, 78.3, 77.9, 61.0, 60.4, 60.3, 60.2, 27.8 (2 signals), 21.8, 21.5, 20.1, 20.0, 15.5, 15.3; IR (film, υ cm⁻¹): 1710, 1274, 1160; HRMS (ESI) calc. for C₁₃H₂₂NaO₄ [M+Na]⁺: 265.14103. obs. 265.14100.

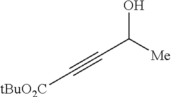 [SMILES: CC(O)C#CC(=O)OC(C)(C)C]

### tert-butyl 4-hydroxypent-2-ynoate

To a stirred solution of diasteromeric esters (12) (117 g, 483 mmol, 1.0 equiv.) in EtOH (4.8 L, 0.1 M) at 78° C. was added solid pyridinium p-toluenesulfonate (12.1 g, 48.3 mmol, 0.1 equiv.). After 2 hours, the reaction mixture was allowed to cool to 23° C. The reaction mixture was diluted with Et₂O (2.4 L) and washed with brine (4 L). The organic layer was dried over MgSO₄ and concentrated in vacuo to give alcohol X (73.1 g, 429 mmol, 89%) as an amber oil.

R_{f}=0.30 (silica gel, 3:1 hexanes:EtOAc); ¹H NMR (400 MHz, CDCl₃): δ 4.62 (m, 1H), 2.13 (s, 1H), 1.51 (m, 12H); ¹³C NMR (100 MHz, CDCl₃): δ 152.8, 86.8, 82.9, 77.5, 57.8, 27.8, 23.1; IR (film, υ cm⁻¹): 3400, 1709; HRMS (EC-CI) calc. for C₉H₁₅O₃ [M+H]⁺: 171.1021. obs. 171.1019.

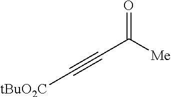 [SMILES: CC(=O)C#CC(=O)OC(C)(C)C]

### tert-butyl 4-oxopent-2-ynoate (8)

To a stirred solution of alcohol (73.0 g, 429 mmol, 1.0 equiv.) in Me₂CO (1.2 L, 0.43 M) at 0° C. was slowly added ice-cold 1.53 M (67.0 g CrO₃, 58.0 mL conc. H₂SO₄ and 160 mL H₂O) Jones reagent (280 mL, 429 mmol, 1.0 equiv.) over 15 minutes. After 30 minutes, i-PrOH (40 mL) was added to neutralize any excess Jones reagent and the reaction mixture was diluted with CH₂Cl₂ (1 L). The organic layer was decanted and washed with H₂O (1 L), sat. aq. NaHCO₃ (1 L) and brine (1 L). The organic layer was dried over Na₂SO₄ and concentrated in vacuo to give keto ester 8 (57.5 g, 342 mmol, 80%) as a clear amber oil.

R_{f}=0.40 (silica gel, 10:1 hexanes:EtOAc); ¹H NMR (400 MHz, CDCl₃): δ 2.41 (s, 3H), 1.52 (s, 9H); ¹³C NMR (100 MHz, CDCl₃): δ 182.8, 151.0, 85.4, 79.2, 79.0, 32.3, 27.9; IR (film, υ cm⁻¹): 1716, 1689; HRMS (EC-CI) calc. for C₉H₁₃O₃ [M+H]⁺: 169.0865. obs. 169.0866.

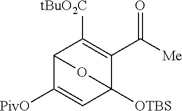

### tert-butyl 3-acetyl-4-((tert-butyldimethlsilyl)oxy)-6-(pivaloyloxy)-7-oxabicyclo[2.2.1]hepta-2,5-diene-2-carboxylate (9)

To a stirred solution of furan (7) (70.4 g, 236 mmol, 1.0 equiv.) in THF (212 mL, 1.1 M) at 0° C. was added keto ester (8) (39.7 g, 236 mmol, 1.0 equiv.). Upon complete addition the reaction mixture was allowed to warm to 23° C. After 1 hour, the reaction mixture was concentrated in vacuo to give bicycle (9) (110 g, 236 mmol, yield taken after subsequent step) in >20:1 regioselectivity as a viscous burgundy oil.

R_{f}=0.35 (silica gel, 10:1 hexanes:EtOAc); ¹H NMR (400 MHz, CDCl₃): δ 6.38 (s, 1H), 5.24 (s, 1H), 2.43 (s, 3H), 1.47 (s, 9H), 1.25 (s, 9H), 0.90 (s, 9H), 0.20 (s, 3H), 0.18 (s, 3H); ¹³C NMR (100 MHz, CDCl₃): δ 199.3, 174.3, 167.7, 163.7, 161.2, 146.3, 118.5, 113.9, 82.3, 78.2, 39.2, 30.7, 27.9, 26.8, 25.4, 17.7, −3.5, −3.7; IR (film, υ cm⁻¹): 1769, 1712; HRMS (EC-CI) calc. for C₂₄H₃₈O₇Si [M+Na]⁺: 489.22790. obs. 489.22801.

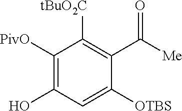

### tert-butyl 2-acetyl-3-((tert-butyldimethlsilyl)oxy)-5-hydroxy-6-(pivaloyloxy)benzoate (13)

To a stirred solution of bicycle (9) (110 g, 236 mmol, 1.0 equiv.) in THF (471 mL, 0.5 at 0° C. was slowly added a 4.0 M solution of hydrochloric acid in dioxane (47.1 mL, 47.1 mmol, 0.2 equiv.) over 5 minutes. Upon complete addition the reaction mixture was allowed to warm to 23° C. After 2 hours, the reaction mixture was concentrated in vacuo to give an amber oil. The crude material was purified via silica gel column chromatography (20:1 hexanes:EtOAc) to give pure phenol 13 (82.9 g, 178 mmol, 75% over 2-steps) as a clear light-yellow oil.

R_{f}=0.38 (silica gel, 10:1 hexanes:EtOAc); ¹H NMR (400 MHz, CDCl₃): δ 10.91 (s, 1H), 6.71 (s, 1H), 2.48 (s, 3H), 1.54 (s, 9H), 1.38 (s, 9H), 0.94 (s, 9H), 0.18 (s, 9H); ¹³C NMR (100 MHz, CDCl₃): δ 202.3, 176.3, 168.4, 148.7, 142.5, 139.7, 131.9, 119.9, 111.0, 85.7, 39.2, 32.5, 27.8, 27.2, 25.5, 18.0, −4.4; IR (film, υ cm⁻¹): 1763, 1716, 1673; HRMS (EC-CI) calc. for C₂₄H₃₈O₇Si [M+Na]⁺: 489.22790. obs. 489.22813.

B. General Procedure for Methoxymethyl Ether Protection

To a stirred solution of phenol (1.0 equiv.) and N,N-diisopropylethylamine (1.5 equiv.) in CH₂Cl₂ (0.2 M) at 0° C. was added and a 2.1 M solution of methoxymethyl chloride in PhMe/MeOAc (1.5 equiv.). After 1 hour, the reaction mixture was diluted with 0.1 M aq. HCl and extracted with CH₂Cl₂ (3×). The combined organic layers were dried over Na₂SO₄ and concentrated in vacuo to give crude methoxymethyl ether. The crude material was purified via silica gel column chromatograph to give pure methoxymethyl ether.

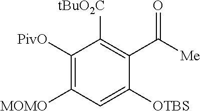

### tert-butyl 2-acetyl-3-((tert-butyldimethlsilyl)oxy)-5-(methoxymethoxy)-6-(pivaloyloxy)benzoate (14)

Following the general procedure for methoxymethyl ether protection, phenol (13) was transformed into methoxymethyl ether (14). The crude material was purified via silica gel column chromatography (10:1 hexanes:EtOAc) to give pure methoxymethyl ether (14) (61.4 g, 120 mmol, 68%) as a white solid (m.p. 60-62° C.). R_{f}=0.61 (silica gel, 3:1 hexanes:EtOAc); ¹H NMR (400 MHz, CDCl₃): δ 6.76 (s, 1H), 5.10 (s, 2H), 3.42 (s, 3H), 2.54 (s, 3H), 1.49 (s, 9H), 1.34 (s, 9H), 0.97 (s, 9H), 0.21 (s, 9H); ¹³C NMR (100 MHz, CDCl₃): δ 200.9, 175.7, 163.5, 150.9, 150.4, 132.8, 128.1, 125.7, 108.6, 94.6, 82.5, 55.9, 38.9, 31.7, 27.7, 27.1, 25.6, 18.1, −4.4; IR (film, υ cm⁻¹): 1761, 1733, 1703; HRMS (ESI) calc. for C₂₆H₄₂NaO₈Si [M+Na]⁺: 533.25412. obs. 533.25387.

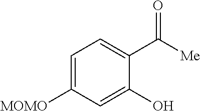

### 1-(2-hydroxy-4-(methoxymethoxy)phenyl)ethan-1-one (24)

Following the general procedure for methoxymethyl ether protection, 2′,4′-dihydroxyacetophenone (23) was transformed into methoxymethyl ether (24). The crude material was purified via silica gel column chromatography (10:1 hexanes:EtOAc) to give pure methoxymethyl ether (24) (8.84 g, 45.1 mmol, 69%) as a clear oil.

R_{f}=0.45 (silica gel, 5:1 hexanes:EtOAc); ¹H NMR (400 MHz, CDCl₃): δ 12.62 (s, 1H), 7.66 (d, J=8.9 Hz, 1H), 6.60 (d, J=2.4 Hz, 1H), 6.55 (dd, J=8.9, 2.4 Hz, 1H), 5.21 (s, 2H), 3.48 (s, 3H), 2.57 (s, 3H); ¹³C NMR (100 MHz, CDCl₃): δ 202.7, 164.7, 163.5, 132.4, 114.6, 108.1, 103.6, 93.9, 56.3, 26.1; IR (film, υ cm⁻¹): 3406, 1635, 1244, 991; HRMS (EC-CI) calc. for C₁₀H₁₃O₄ [M+H]⁺: 197.0814. obs. 197.0814.

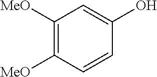 [SMILES: COc1ccc(O)cc1OC]

### 3,4-dimethoxyphenol

To a stirred solution of 3,4-dimethoxybenzaldehyde (26) (30.0 g, 181 mmol, 1.0 equiv.) in CH₂Cl₂ (361 mL, 0.5 M) at 23° C. was added 30% aq. H₂O₂ (46.1 mL, 451 mmol, 2.5 equiv.) and formic acid (27.7 mL, 722 mmol, 4.0 equiv.). The reaction mixture was stirred at 40° C. for 42.5 hours. The reaction mixture was then cooled to 23° C. and the organic layer was separated. The aqueous layer was extracted with CH₂Cl₂ (3×50 mL) and the combined organic layers were dried over Na₂SO₄ and concentrated in vacuo to about 361 mL (0.5 M). 5 M aq. NaOH (251 mL, 1.26 mol, 10 equiv.) was then slowly added and the reaction mixture was stirred at 23° C. for 20 minutes. The organic layer was separated and the aqueous layer was washed with CH₂Cl₂ (3×100 mL). The aqueous layer was acidified to pH=1.0 with conc. HCl and extracted with CH₂Cl₂ (3×100 mL). The combined organic layers were dried over Na₂SO₄ and concentrated in vacuo to give pure 3,4-dimethoxyphenol (19.05 g, 124 mmol, 68%) as an amber solid (m.p. 58-60° C.).

R_{f}=0.43 (silica gel, 1:1 hexanes:EtOAc); ¹H NMR (400 MHz, CDCl₃): δ 6.71 (d, J=8.4 Hz, 1H), 6.46 (d, J=2.7 Hz, 1H), 6.35 (dd, J=8.4, 2.7 Hz, 1H), 5.93 (bs, 1H), 3.79 (s, 3H), 3.76 (s, 3H); ¹³C NMR (100 MHz, CDCl₃): δ 150.2, 149.7, 142.8, 112.5, 105.9, 100.6, 56.5, 55.6; IR (film, υ cm⁻¹): 3382, 1513, 1223; HRMS (EC-CI) calc. for C₈H₁₁O₃ [M+H]⁺: 155.0708. obs. 155.0700.

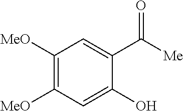 [SMILES: COc1cc(O)c(C(C)=O)cc1OC]

### 1-(2-hydroxy-4,5-dimethoxyphenyl)ethan-1-one (27)

To a stirred solution of 3,4-dimethoxyphenol (3.0 g, 19.5 mmol, 1 equiv.) in acetic anhydride (9.75 mL, 103 mmol, 5.3 equiv.) at 0° C. was added neat boron trifluoride diethyl etherate (4.8 mL, 38.9 mmol, 2 equiv.). The reaction mixture was stirred at 90° C. for 1 hour and then allowed to sit at 23° C. for 16 hours. The precipitate was collected and recrystallized from EtOH to give pure hydroxyacetophenone (27) (3.38 g, 17.7 mmol, 89%) as white needles (m.p. 104-105° C.).

R_{f}=0.58 (silica gel, 1:1 hexanes:EtOAc); ¹H NMR (400 MHz, CDCl₃): δ 12.65 (s, 1H), 7.05 (s, 1H), 6.46 (s, 1H), 3.91 (s, 3H), 3.87 (s, 3H), 2.56 (s, 3H); ¹³C NMR (100 MHz, CDCl₃): δ 202.0, 160.0, 156.7, 141.8, 111.6, 111.5, 100.5, 56.6, 56.1, 26.3; IR (film, υ cm⁻¹): 1632, 1511, 1265, 1160, 1063; HRMS (EC-CI) calc. for C₁₀H₁₃O₄ [M+H]⁺: 197.0814. obs. 197.0810.

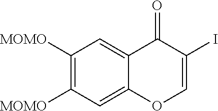

### 3-iodo-6,7-bis(methoxymethoxy)-4H-chromen-4-one (28)

Following the general procedure for methoxymethyl ether protection, catechol was transformed into iodochromone (28). In this case, 3.0 equivalents of N,N-diisopropylethylamine and 3.0 equivalents fo methoxymethyl chloride were used. The crude material was purified via silica gel column chromatography (2:1 hexanes:EtOAc) to give pure methoxymethyl ether (28) (417 mg, 1.06 mmol, 71%) as a white solid (m.p. 105-106° C.).

R_{f}=0.29 (silica gel, 2:1 hexanes:EtOAc); ¹H NMR (400 MHz, CDCl₃): δ 8.19 (s, 1H), 7.79 (s, 1H), 7.17 (s, 1H), 5.31 (s, 2H), 5.27 (s, 2H), 3.50 (s, 3H), 3.48 (s, 3H); ¹³C NMR (100 MHz, CDCl₃): δ 172.2, 157.0, 152.7, 152.4, 145.5, 116.0, 110.8, 103.5, 95.4, 95.1, 86.3, 56.5, 56.3; IR (film, υ cm⁻¹): 1617, 1453, 1284, 1152, 1041; HRMS (ESI) calc. for C₁₃H₁₃INaO₆ [M+Na]⁺: 414.96490. obs. 414.96555.

C. General Procedure for Enaminone Formation

To a stirred solution of hydroxyacetophenone (1.0 equiv.) in dimethoxyethane (0.5 M) at 85° C. was added N,N-dimethylformamide dimethyl acetal (3.0 equiv.). After 4 hours, the reaction mixture was cooled to 23° C. and then concentrated in vacuo to give crude enaminone of sufficient purity for subsequent reactions.

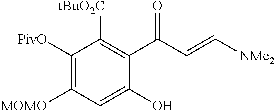

### tert-butyl (E)-2-(3-(dimethylamino)acryloyl)-3-hydroxy-5-(methoxymethoxy)-6-(pivaloyloxy)benzoate (15)

Following the general procedure for enaminone formation, acetophenone (14) was transformed into enaminone (15) (yield taken after subsequent step), an orange solid (m.p. 118-119° C.) of sufficient purity for subsequent reactions.

R_{f}=0.26 (silica gel, 1:1 hexanes:EtOAc); ¹H NMR (400 MHz, CDCl₃): δ 12.43 (bs, 1H), 7.77 (d, J=12 Hz, 1H), 6.70 (s, 1H), 5.49 (d, J=12 Hz, 1H), 5.13 (s, 2H), 3.41 (s, 3H), 3.15 (s, 3H), 2.84 (s, 3H), 1.47 (s, 9H), 1.34 (s, 9H); ¹³C NMR (100 MHz, CDCl₃): δ 189.4, 175.8, 165.6, 159.3, 154.4, 151.6, 130.1, 128.5, 113.7, 104.0, 95.2, 94.0, 82.4, 56.0, 45.1, 38.7, 37.1, 27.6, 27.0; IR (film, υ cm⁻¹): 1751, 1716, 1632, 1111; HRMS (ESI) calc. for C₂₃H₃₃NNaO₈ [M+Na]⁺: 474.20984. obs. 474.21058.

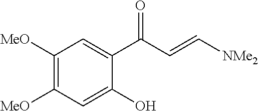 [SMILES: COc1cc(O)c(C(=O)/C=C/N(C)C)cc1OC]

### (E)-3-(dimethylamino)-1-(2-hydroxy-4,5-(dimethoxyphenyl)prop-2-en-1-one

Following the general procedure for enaminone formation, acetophenone (27) was transformed into enaminone (yield taken after subsequent step), a yellow solid (m.p. 157-158° C.) of sufficient purity for subsequent reactions.

R_{f}=0.18 (silica gel, 1:1 hexanes:EtOAc); ¹H NMR (600 MHz, CDCl₃): δ 14.25 (bs, 1H), 7.84 (d, J=12 Hz, 1H), 7.10 (s, 1H), 6.44 (s, 1H), 5.60 (d, J=12 Hz, 1H), 3.88 (s, 3H), 3.85 (s, 3H), 3.16 (bs, 3H), 2.96 (bs, 3H); ¹³C NMR (150 MHz, CDCl₃): δ 190.3, 160.1, 155.0, 154.1, 141.2, 111.7, 111.1, 100.8, 89.7, 57.1, 55.9, 45.3, 37.3; IR (film, υ cm⁻¹): 1630, 1543, 1376, 1228, 1113; HRMS (ESI) calc. for C₁₃H₁₈NO₄ [M+H]⁺: 252.12303. obs. 252.12258.

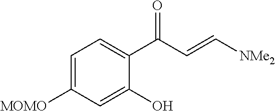

### (E)-3-(dimethylamino)-1-(2-hydroxy-4-(methoxymethoxy)phenyl)prop-2-en-1-one

Following the general procedure for enaminone formation, acetophenone (24) was transformed into enaminone (yield taken after subsequent step), a yellow solid (m.p. 95-96° C.) of sufficient purity for subsequent reactions.

R_{f}=0.25 (silica gel, 1:1 hexanes:EtOAc); ¹H NMR (400 MHz, CDCl₃): δ 7.85 (d, J=12 Hz, 1H), 7.62 (d, J=8.9 Hz, 1H), 6.58 (d, J=2.4 Hz, 1H), 6.48 (dd, J=8.9, 2.4 Hz, 1H), 5.69 (d, J=12 Hz, 1H), 5.19 (s, 2H), 3.47 (s, 3H), 3.18 (bs, 3H), 2.96 (bs, 3H); ¹³C NMR (100 MHz, CDCl₃): δ 190.4, 165.0, 161.6, 154.1, 129.6, 114.8, 106.9, 103.8, 93.9, 89.6, 56.1, 45.2, 37.2; IR (film, υ cm⁻¹): 1627, 1535, 1235, 1108; HRMS (ESI) calc. for C₁₃H₁₇NNaO₄ [M+Na]⁺: 274.10498. obs. 274.10491.

D. General Procedure for Iodochromone Formation

To a stirred solution of crude enaminone (1.0 equiv.) in CHCl₃ (0.1 M) at 23° C. was added solid iodine (2.0 equiv.). After 1 hour, the reaction mixture was diluted with sat. aq. Na₂S₂O₃ and extracted with CH₂Cl₂. The organic layer was dried over Na₂SO₄ and concentrated in vacuo to give crude enaminone. The crude material was purified via silica gel column chromatography to give pure iodochromone.

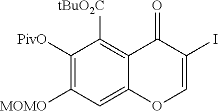

### tert-butyl 3-iodo-7-(methoxymethoxy)-4-oxo-6-(pivaloyloxy)-4H-chromene-5-carboxylate (18)

Following the general procedure for iodochromone formation, enaminone (15) was transformed into iodochromone (18). The crude material was purified via silica gel column chromatography (1:1 hexanes:EtOAc) to give pure iodochromone (18) (9.65 g, 18.1 mmol, 60% over 2-steps) as a white solid (m.p. 189-190° C.).

R_{f}=0.32 (silica gel, 3:1 hexanes:EtOAc); ¹H NMR (400 MHz, CDCl₃): δ 8.19 (s, 1H), 7.17 (s, 1H), 5.23, (s, 2H), 3.25 (s, 3H), 1.64 (s, 9H), 1.37 (s, 9H); ¹³C NMR (100 MHz, CDCl₃): δ 175.4, 170.9, 163.2, 156.8, 154.9, 153.3, 136.5, 128.3, 112.8, 103.5, 94.7, 86.7, 83.3, 56.6, 39.2, 28.2, 27.2; IR (film, υ cm⁻¹): 1764, 1731, 1650; HRMS (ESI) calc. for C₂₁H₂₅INaO₈ [M+Na]⁺: 555.04863. obs. 555.04881.

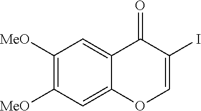 [SMILES: COc1cc2occ(I)c(=O)c2cc1OC]

### 3-iodo-6,7-dimethoxy-4H-chromen-4-one

Following the general procedure for iodochromone formation, enaminone ((E)-3-(dimethylamino)-1-(2-hydroxy-4,5-(dimethoxyphenyl)prop-2-en-1-one) was transformed into the iodochromone. The crude material was purified via silica gel column chromatography (2:1 hexanes:EtOAc) to give pure iodochromone (7.01 g, 21.1 mmol, 35% over 2-steps) as a white solid (m.p. 170-172° C.).

R_{f}=0.32 (silica gel, 2:1 hexanes:EtOAc); ¹H NMR (400 MHz, CDCl₃): δ 8.24 (s, 1H), 7.55 (s, 1H), 6.86 (s, 1H), 3.99 (s, 3H), 3.98 (s, 3H); ¹³C NMR (100 MHz, CDCl₃): δ 172.2, 156.7, 154.5, 152.1, 147.9, 115.0, 104.8, 99.3, 86.4, 56.4, 56.3; IR (film, υ cm⁻¹): 1615, 1505, 1471, 1289, 1226; HRMS (ESI) calc. for C₁₁H₉INaO₄ [M+Na]⁺: 354.94377. obs. 354.94418.

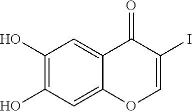 [SMILES: O=c1c(I)coc2cc(O)c(O)cc12]

### 6,7-dihydroxy-3-iodo-4H-chromen-4-one

To a stirred solution of iodochromone (3-iodo-6,7-dimethoxy-4H-chromen-4-one) (500 mg, 1.51 mmol, 1.0 equiv.) in CH₂Cl₂ (15.1 mL, 0.1 M) at 0° C. was slowly added neat boron tribromide (0.86 mL, 9.03 mmol, 6.0 equiv.). The reaction mixture was stirred at 23° C. for 1.5 hours. The reaction mixture was then carefully quenched with 1.25 M methanolic HCl (1.2 mL, 2.08 mmol, 1.0 equiv.) at 0° C. and stirred for 5 minutes. The reaction mixture was purged with N₂ and concentrated in vacuo to give the iodochromone (458 mg, 1.51 mmol, 99%) as a grey solid (m.p. 215° C. (decomp.)) of sufficient purity for subsequent reactions.

R_{f}=0.72 (silica gel, 20:1 EtOAc:AcOH); ¹H NMR (400 MHz, CD₃OD): δ 8.49 (s, 1H), 7.40 (s, 1H), 6.89 (s, 1H); ¹³C NMR (100 MHz, CD₃OD): δ 174.8, 159.6, 154.5, 153.4, 146.6, 115.6, 108.9, 103.5, 85.4; IR (KBr, υ cm⁻¹): 3218, 1616, 1471, 1308; HRMS (EC-CI) calc. for C₉H₆O₄ [M+H]⁺: 304.9311. obs. 304.9308.

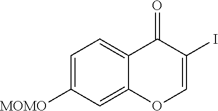

### 3-iodo-7-(methoxymethoxy)-4H-chromen-4-one (25)

Following the general procedure for iodochromone formation, enaminone ((E)-3-(dimethylamino)-1-(2-hydroxy-4-(methoxymethoxy)phenyl)prop-2-en-1-one) was transformed into iodochromone (25). The crude material was purified via silica gel column chromatography (3:1 hexanes:EtOAc) to give pure iodochromone (25) (11.69 g, 35.2 mmol, 78% over 2-steps) as a white solid (m.p. 101-102° C.).

R_{f}=0.28 (silica gel, 5:1 hexanes:EtOAc); ¹H NMR (400 MHz, CDCl₃): δ 8.23 (s, 1H), 8.17 (d, J=8.6 Hz, 1H), 7.10 (dd, J=8.9, 2.4 Hz, 1H), 7.08 (d, J=2.1 Hz, 1H), 5.27 (s, 2H), 3.50 (s, 3H); ¹³C NMR (100 MHz, CDCl₃): δ 172.4, 161.6, 157.4, 157.2, 127.8, 116.2, 116.1, 102.8, 94.2, 86.9, 56.4; IR (film, υ cm⁻¹): 1646, 1624, 1149; HRMS (ESI) calc. for C₁₁H₉INaO₄ [M+Na]⁺: 354.94377. obs. 354.94436.

E. General Procedure for Propargyl Alcohol Formation

To a stirred solution of iodochromone (1.0 equiv.), bis(triphenylphosphine) palladium (II) dichloride (0.02 equiv.) and copper (I) iodide (0.1 equiv.) in freeze pump thawed THF (0.1 M) at 23° C. was added 3-butyn-2-ol (11) (4.0 equiv.). Diisopropylamine (3.0 equiv.) was then added. After 1 hour, the reaction mixture was diluted with pH=7.0 phosphate buffer and extracted with CH₂Cl₂. The organic layer was dried over Na₂SO₄ and concentrated in vacuo to give crude propargyl alcohol. The crude material was purified via silica gel column chromatography to give pure propargyl alcohol.

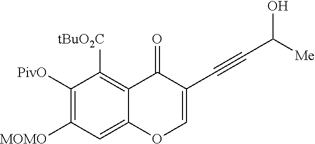

### tert-butyl 3-(3-hydroxybut-1-yn-1-yl)-7-(methoxymethoxy)-4-oxo-6-(pivaloyloxy)-4H-chromene-5-carboxylate

Following the general procedure for propargyl alcohol formation, iodochromone (18) was transformed into the propargyl alcohol. The crude material was purified via silica gel column chromatography (1:1 hexanes:EtOAc) to give pure propargyl alcohol (1.71 g, 3.60 mmol, 64%) as a tan solid (m.p. 132-134° C.).

R_{f}=0.21 (silica gel, 1:1 hexanes:EtOAc); ¹H NMR (400 MHz, CDCl₃): δ 8.03 (s, 1H), 7.14 (s, 1H), 5.21 (s, 2H), 4.75 (q, J=6.7 Hz, 1H), 3.43 (s, 3H), 3.20 (bs, 1H), 1.63 (s, 9H), 1.51 (d, J=6.7 Hz, 3H); ¹³C NMR (100 MHz, CDCl₃): δ 175.5, 173.3, 163.3, 157.5, 154.6, 153.2, 136.3, 128.1, 114.5, 110.5, 103.8, 97.5, 94.6, 83.2, 73.8, 58.6, 56.6, 39.2, 28.2, 27.2, 23.8; IR (film, υ cm⁻¹): 3435, 1763, 1735, 1731, 1461; HRMS (ESI) calc. for C₂₅H₃₀NaO₉ [M+Na]⁺: 497.1782. obs. 497.1785.

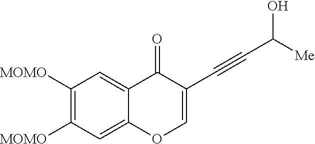

### 3-(3-hydroxybut-1-yn-1-yl)-6,7-bis(methoxymethoxy)-4H-chromen-4-one

Following the general procedure for propargyl alcohol formation, iodochromone (28) was transformed into propargyl alcohol. The crude material was purified via silica gel column chromatography (1:1 to 1:2 hexanes:EtOAc) to give pure propargyl alcohol (970 mg, 2.90 mmol, 81%) as an amber oil.

R_{f}=0.12 (silica gel, 1:1 hexanes:EtOAc); ¹H NMR (400 MHz, CDCl₃): δ 8.06 (s, 1H), 7.82 (s, 1H), 7.20 (s, 1H), 5.33 (s, 2H), 5.30 (s, 2H), 4.79 (q, J=6.7 Hz, 1H), 3.52 (s, 3H), 3.51 (s, 3H), 3.30 (bs, 1H), 1.54 (d, J=6.7 Hz, 3H); ¹³C NMR (100 MHz, CDCl₃): δ 174.7, 157.6, 152.7, 152.3, 145.5, 117.8, 110.4, 109.9, 103.9, 97.2, 95.5, 95.1, 74.2, 58.6, 56.6, 56.5, 24.0; IR (film, υ cm⁻¹): 3397, 1621, 1494, 1460, 1266, 1227, 986; HRMS (ESI) calc. for C₁₇H₁₈NaO₇ [M+Na]⁺: 357.09447. obs. 357.09487.

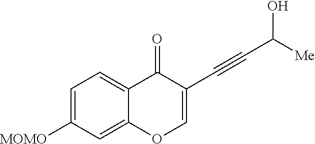

### 3-(3-hydroxybut-1-yn-1-yl)-7-(methoxymethoxy)-4H-chromen-4-one

Following the general procedure for propargyl alcohol formation, iodochromone (25) was transformed into propargyl alcohol. The crude material was purified via silica gel column chromatography (1:1 hexanes:EtOAc) to give pure propargyl alcohol (696 mg, 2.54 mmol, 84%) as an amber oil.

R_{f}=0.28 (silica gel, 1:1 hexanes:EtOAc); ¹H NMR (400 MHz, CDCl₃): δ 8.16 (dd, J=7.9, 1.0 Hz, 1H), 8.09 (s, 1H), 7.10 (d, J=2.4 Hz, 1H), 7.09 (d, J=1.0 Hz, 1H), 5.27 (s, 2H), 4.79 (q, J=6.8 Hz, 1H), 3.50 (s, 3H), 2.43 (bs, 1H), 1.56 (d, J=6.8 Hz, 3H); ¹³C NMR (100 MHz, CDCl₃): δ 175.0, 161.7, 157.8, 157.3, 127.5, 117.8, 115.9, 110.6, 103.1, 97.6, 94.3, 73.9, 58.4, 56.4, 23.9; IR (film, υ cm⁻¹): 3392, 1624, 1249, 1077; HRMS (ESI) calc. for C₁₅H₁₄NaO₅ [M+Na]⁺: 297.07334. obs. 297.07349.

F. General Procedure for Ynone Formation

To a stirred solution of propargyl alcohol (1.0 equiv.) and activated 4.0 Å molecular sieves (50% by weight) in CH₂Cl₂ (0.1 M) at 23° C. was added solid pyridinium dichromate (5.0 equiv.). After 5 hours, the reaction mixture was filtered through a pad of Celite and concentrated in vacuo to give crude ynone. The crude material was purified via silica gel column chromatography to give pure ynone.

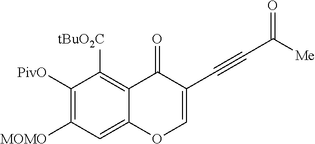

### tert-butyl 7-(methoxymethoxy)-4-oxo-3-(3-oxobut-1-yn-1-yl)-6-(pivaloyloxy)-4H-chromene-5-carboxylate (19)

Following the general procedure for ynone formation, propargyl alcohol (tert-butyl 3-(3-hydroxybut-1-yn-1-yl)-7-(methoxymethoxy)-4-oxo-6-(pivaloyloxy)-4H-chromene-5-carboxylate) was transformed into ynone (19). The crude material was purified via silica gel column chromatography (1:1 hexanes:EtOAc) to give pure ynone (19) (2.79 g, 5.90 mmol, 56%) as a white solid (m.p. 182-183° C.).

R_{f}=0.41 (silica gel, 1:1 hexanes:EtOAc); ¹H NMR (400 MHz, CDCl₃): δ 8.20 (s, 1H), 7.21 (s, 1H), 5.24 (s, 2H), 3.44 (s, 3H), 2.46 (s, 3H), 1.64 (s, 9H), 1.37 (s, 9H); ¹³C NMR (100 MHz, CDCl₃): δ 184.2, 175.4, 172.1, 163.1, 160.4, 154.6, 153.7, 136.8, 128.3, 114.6, 108.7, 104.0, 94.7, 93.5, 83.5, 81.0, 56.7, 39.2, 32.7, 28.2, 27.2; IR (film, υ cm⁻¹): 1762, 1734, 1672, 1620, 1459, 1264, 1246, 1155, 1091; HRMS (ESI) calc. for C₂₅H₂₈NaO₉ [M+Na]⁺: 495.1626. obs. 495.1632.

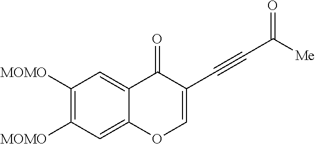

### 6,7-bis(methoxymethoxy)-3-(3-oxbut-1-yn-1-yl)-4H-chromen-4-one (21)

Following the general procedure for ynone formation, propargyl alcohol (3-(3-hydroxybut-1-yn-1-yl)-6,7-bis(methoxymethoxy)-4H-chromen-4-one) was transformed into ynone (21). The crude material was purified via silica gel column chromatography (5:2:1 CH₂Cl₂:EtOAc:hexanes) to give pure ynone (21) (540 mg, 1.63 mmol, 56%) as a white solid (m.p. 119-120° C.).

R_{f}=0.51 (silica gel, 5:2:1 CH₂Cl₂:EtOAc:hexanes); ¹H NMR (400 MHz, CDCl₃): 8.23 (s, 1H), 7.86 (s, 1H), 7.26 (s, 1H), 5.35 (s, 2H), 5.32 (s, 2H), 3.54 (s, 3H), 3.53 (s, 3H), 2.49 (s, 3H); ¹³C NMR (100 MHz, CDCl₃): δ 184.3, 173.5, 160.7, 153.0, 152.2, 146.0, 117.8, 110.4, 108.1, 104.1, 95.5, 95.2, 93.4, 81.8, 56.7, 56.5, 32.7; IR (film, υ cm⁻¹): 1668, 1640, 1615, 1271, 970; HRMS (ESI) calc. for C₁₇H₁₇O₇ [M+H]⁺: 333.09688. obs. 333.09704.

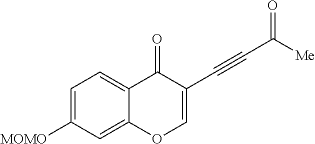

### 7-(methoxymethoxy)-3-(3-oxbut-1-yn-1-yl)-4H-chromen-4-one (22)

Following the general procedure for ynone formation, propargyl alcohol (3-(3-hydroxybut-1-yn-1-yl)-7-(methoxymethoxy)-4H-chromen-4-one) was transformed into ynone (22). The crude material was purified via silica gel column chromatography (3:1 to 2:1 hexanes:EtOAc) to give pure ynone (22) (410 mg, 1.51 mmol, 64%) as a white solid (m.p. 139-141° C.).

R_{f}=0.65 (silica gel, 1:1 hexanes:EtOAc); ¹H NMR (400 MHz, CDCl₃): δ 8.24 (s, 1H), 8.17 (d, J=8.6 Hz, 1H), 7.13 (dd, J=8.6, 2.4 Hz, 1H), 7.11 (d, J=2.1 Hz, 1H), 5.28 (s, 2H), 3.50 (s, 3H), 2.49 (s, 3H); ¹³C NMR (100 MHz, CDCl₃): δ 183.9, 173.5, 162.0, 160.9, 157.1, 127.3, 117.6, 116.3, 108.3, 103.3, 94.2, 93.2, 81.4, 56.3, 32.5; IR (film, υ cm⁻¹): 1669, 1632, 1255, 1158; HRMS (ESI) calc. for C₁₅H₁₂NaO₅ [M+Na]⁺: 295.05769. obs. 295.05778.

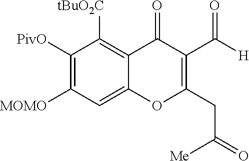

### tert-butyl 3-formyl-7-(methoxymethoxy)-4-oxo-2-(2-oxopropyl)-6-(pivaloyloxy)-4H-chromene-5-carboxylate (20)

To a stirred solution of ynone (19) (100 mg, 0.212 mmol, 1.0 equiv.) and H₂O (3.81 mL, 212 mmol, 1000 equiv.) in MeCN (21.2 mL, 0.01 M) at 23° C. was added triethylamine (0.3 mL, 2.12 mmol, 10 equiv.). After 1 hour, the reaction mixture was diluted with EtOAC (20 mL), dried over Na₂SO₄ and concentrated in vacuo to give aldehyde (20) (104 mg, 0.212 mmol, 99%) as an amber solid (m.p. 178-179° C. (decomp.)) of sufficient purity for subsequent reactions.

R_{f}=0.23 (silica gel, 1:1 hexanes:EtOAc); ¹H NMR (400 MHz, CDCl₃) δ 10.42 (s, 1H), 7.20 (s, 1H), 5.23 (s, 2H), 4.26 (bs, 2H), 3.45 (s, 3H), 2.38 (s, 3H), 1.64 (s, 9H), 1.39 (s, 9H); ¹³C NMR (125 MHz, CDCl₃) δ 200.0, 190.7, 175.4, 175.0, 168.5, 163.3, 154.5, 153.9, 136.7, 128.2, 117.4, 115.5, 104.1, 94.8, 83.4, 56.6, 47.5, 39.2, 30.4, 28.2, 27.2; IR (film, υ cm⁻¹) 3420, 1762, 1730, 1653, 1595, 1458, 1265, 1157, 1095; HRMS (ESI) calc. for C₂₅H₃₀NaO₁₀ [M+Na]⁺: 513.17312. obs. 513.17312.

G. General Procedure A for Ynone Dimerization

To a stirred solution of ynone (1.0 equiv.) (intended left side of protected vinaxanthone) and H₂O (1000 equiv.) in MeCN (0.01 M) at 23° C. was added triethylamine (10 equiv.). After 1 hour, the reaction mixture was diluted with EtOAc, dried over Na₂SO₄ and concentrated in vacuo to give an amber oil. The crude aldehyde was diluted to 0.1 M with MeCN before ynone (1.0 equiv.) (intended right side of protected vinaxanthone) and triethylamine (2 equiv.) were added. The reaction mixture was stirred at 23° C. for 16 hours. The reaction mixture was then concentrated to give crude protected vinaxanthone. The crude material was purified via silica gel column chromatography to give pure protected vinaxanthone.

H. General Procedure B for Ynone Dimerization

To a stirred solution of ynone (1.0 equiv.) in MeCN (0.1 M) at 23° C. was added a 1.0 M solution of H₂O in MeCN (0.5 equiv.) and triethylamine (10 equiv.). After 16 hours, the reaction mixture was concentrated in vacuo to give crude protected vinaxanthone. The crude material was purified via silica gel column chromatography to give pure protected vinaxanthone.

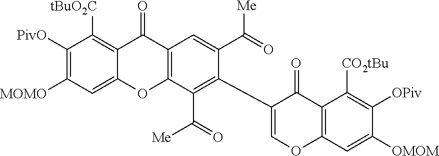

### tert-butyl 5,7-diacetyl-6-(5-(tert-butoxycarbonyl)-7-(methoxymethoxy)-4-oxo-6-(pivaloyloxy)-4H-chromen-3-yl)-3-(methoxymethoxy)-9-oxo-2-(pivaloyloxy)-9H-xanthene-1-carboxylate

Following general procedure B for ynone dimerization, ynone (19) was transformed into the protected vinaxanthone. The crude material was purified via silica gel column chromatography (5:2:1 CH₂Cl₂:EtOAc:hexanes) to give pure protected vinaxanthone (85 mg, 0.090 mmol, 87%) as a white-tan solid (m.p. 224-225° C.).

R_{f}=0.68 (silica gel, 5:2:1 CH₂Cl₂:EtOAc:hexanes); ¹H NMR (400 MHz, CDCl₃) δ 8.62 (bs, 1H), 7.84 (bs, 1H), 7.22 (s, 1H), 7.18 (s, 1H), 5.27 (s, 2H), 5.26 (s, 2H), 3.47 (s, 3H), 3.46 (s, 3H), 2.65 (bs, 3H), 2.41 (bs, 3H), 1.68 (s, 9H), 1.58 (s, 9H), 1.39 (s, 9H), 1.37 (s, 9H); ¹³C NMR (125 MHz, CDCl₃) δ 201.3, 198.8, 175.4 (2 signals), 173.3 (2 signals), 163.4, 163.3, 155.1, 154.6, 154.5, 154.0, 153.5, 152.6, 136.4 (2 signals), 135.9, 133.9, 132.3, 128.9, 128.2, 126.8, 121.2, 120.7, 115.0, 112.7, 103.9, 103.6, 94.7, 94.6, 83.3, 82.8, 56.7, 56.5, 39.2, 39.1, 32.5, 29.6, 28.1, 28.0, 27.2, 27.1; IR (film, υ cm⁻¹) 1763, 1735 1460, 1264, 1157; HRMS (ESI) calc. for C₅₀H₅₆NaO₁₈ [M+Na]⁺: 967.33589. obs. 967.33632.

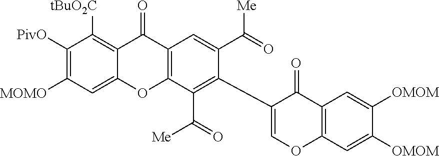

### tert-butyl 5,7-diacetyl-6-(6,7-bis(methoxymethoxy)-4-oxo-4H-chromen-3-yl)-3-(methoxymethoxy)-9-oxo-2-(pivaloyloxy)-9H-xanthene-1-carboxylate (X)

Following general procedure A for ynone dimerization, ynone (19) and ynone (21) were transformed into the protected vinaxanthone. The crude material was purified via silica gel column chromatography (5:2:1 CH₂Cl₂:EtOAc:hexanes) to give pure protected vinaxanthone (37 mg, 0.046 mmol, 23%) as a yellow solid (m.p. 116-118° C.). A side-product protected vinaxanthone (ynone (19) homodimer) (31 mg, 0.047 mmol, 46% with respect to ynone (19)) and another side-product protected vinaxanthone (ynone (21) homodimer) (23 mg, 0.024 mmol, 24% with respect to ynone (21)) were also isolated.

R_{f}=0.51 (silica gel, 5:2:1 CH₂Cl₂:EtOAc:hexanes); ¹H NMR (400 MHz, CDCl₃) δ 8.67 (bs, 1H), 7.98 (s, 1H), 7.84 (bs, 1H), 7.26 (s, 1H), 7.22 (s, 1H), 5.39 (s, 2H), 5.35 (s, 2H), 5.26 (s, 2H), 3.57 (s, 3H), 3.56 (s, 3H), 3.47 (s, 3H), 2.67 (bs, 3H), 2.42 (bs, 3H), 1.58 (s, 9H), 1.37 (s, 9H); ¹³C NMR (125 MHz, CDCl₃) δ 201.8, 199.0, 175.5, 174.5, 173.5, 163.4, 155.2, 154.2, 153.9, 153.6, 153.1, 152.3, 145.1, 136.4, 134.1, 131.8, 128.2, 126.9, 121.4, 120.6, 115.8, 115.1, 111.4, 110.5, 103.9, 103.8, 95.7, 95.2, 94.7, 82.8, 56.7, 56.6, 56.5, 39.2, 32.5, 28.9, 28.2, 27.3; IR (film, υ cm⁻¹) 1654, 1459, 1268, 1156, 1092; HRMS (ESI) calc. for C₄₂H₄₄NaO₁₆ [M+Na]⁺: 827.25220. obs. 827.25320.

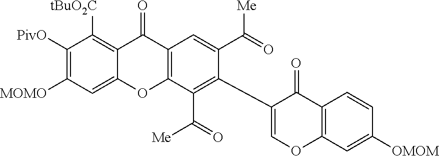

### tert-butyl 5,7-diacetyl-3-(methoxymethoxy)-6-(7-(methoxymethoxy)-4-oxo-4H-chromen-3-yl)-9-oxo-2-(pivaloyloxy)-9H-xanthene-1-carboxylate

Following general procedure A for ynone dimerization, ynone (19) and ynone (22) were transformed into the protected vinaxanthone. The crude material was purified via silica gel column chromatography (5:2:1 CH₂Cl₂:EtOAc:hexanes) to give pure protected vinaxanthone (86 mg, 0.115 mmol, 56%) as a pale off-white solid (m.p. 138-139° C.). A side-product protected vinaxanthone (ynone (19) homodimer) (22 mg, 0.040 mmol, 39% with respect to ynone (19)) and another side-product protected vinaxanthone (ynone (22) homodimer) (10 mg, 10 mol, 10% with respect to ynone (22)) were also isolated.

R_{f}=0.65 (silica gel, 5:2:1 CH₂Cl₂:EtOAc:hexanes); ¹H NMR (500 MHz, CDCl₃) δ 8.64 (bs, 1H), 8.24 (d, J=8.8 Hz, 1H), 7.84 (bs, 1H), 7.21 (s, 1H), 7.09 (d, J=2.3 Hz, 1H), 7.06 (dd, J=8.8, 2.3 Hz, 1H), 5.28 (s, 2H), 5.24 (s, 2H), 3.50 (s, 3H), 3.45 (s, 3H), 2.65 (bs, 3H), 2.41 (bs, 3H), 1.56 (s, 9H), 1.36 (s, 9H); ¹³C NMR (125 MHz, CDCl₃) δ 201.6, 198.8, 175.4, 174.7, 173.4, 163.3, 163.1, 157.3, 155.1, 153.9, 153.5, 153.1, 136.4, 136.0, 134.1, 132.1, 128.4, 128.1, 126.9, 121.3, 121.0, 116.2, 115.3, 115.0, 103.9, 103.1, 94.7, 94.4, 82.7, 56.5 (2 signals), 39.1, 32.4, 28.8, 28.1, 27.2; IR (film, υ cm⁻¹) 1620, 1460, 1262, 1158, 1096; HRMS (ESI) calc. for C₄₀H₄₀NaO₁₄ [M+Na]⁺: 767.23103. obs. 767.23148.

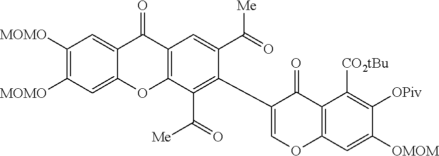

### tert-butyl 3-(2,4-diacetyl-6,7-bis(methoxymethoxy)-9-oxo-9H-xanthen-3-yl)-7-(methoxymethoxy)-4-oxo-6-(pivaloyloxy)-4H-chromene-5-carboxylate

Following general procedure A for ynone dimerization, ynone (21) and ynone (19) were transformed into the protected vinaxanthone. The crude material was purified via silica gel column chromatography (5:2:1 CH₂Cl₂:EtOAc:hexanes) to give pure protected vinaxanthone (107 mg, 0.140 mmol, 55%) as a yellow solid (m.p. 152-154° C.). A side-product protected vinaxanthone (ynone (19) homodimer) (29 mg, 0.031 mmol, 24% with respect to ynone (19)) was also isolated.

R_{f}=0.49 (silica gel, 5:2:1 CH₂Cl₂:EtOAc:hexanes); ¹H NMR (400 MHz, CDCl₃) δ 8.68 (s, 1H), 7.83 (s, 1H), 7.82 (s, 1H), 7.26 (s, 1H), 7.19 (s, 1H), 5.37 (s, 2H), 5.31 (q, J=4.5 Hz, 2H)*, 5.27 (q, J=3.4 Hz, 2H)*, 3.54 (s, 3H), 3.53 (s, 3H), 3.47 (s, 3H), 2.64 (s, 3H), 2.45 (s, 3H), 1.69 (s, 9H), 1.39 (s, 9H); ¹³C NMR (150 MHz, CDCl₃) δ 201.3, 199.3, 175.5, 174.6, 173.4, 163.5, 154.7, 154.6, 153.9, 152.9, 152.7, 145.6, 136.3, 135.9, 133.9, 133.3, 129.0, 127.4, 122.1, 120.7, 118.1, 112.8, 110.6, 104.1, 103.9, 103.8, 103.7, 95.6, 95.1, 94.8, 83.3, 56.7, 56.5, 39.2, 32.4, 28.9, 28.2, 27.3; IR (film, υ cm⁻¹) 1458, 1155, 1090; HRMS (ESI) calc. for C₄₂H₄₄NaO₁₆ [M+Na]⁺: 827.25220. obs. 827.25350. *Non-equivalent methylene protons.

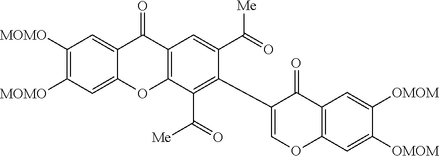

### 1,1′-(3-(6,7-bis(methoxymethoxy)-4-oxo-4H-chromen-3-yl)-6,7-bis(methoxymethoxy)-9-oxo-9H-xanthene-2,4-diyl)bis(ethan-1-one)

Following general procedure B for ynone dimerization, ynone (21) was transformed into the protected vinaxanthone. The crude material was purified via silica gel column chromatography (5:2:1 CH₂Cl₂:EtOAc:hexanes) to give pure protected vinaxanthone (52 mg, 0.078 mmol, 52%) as a yellow solid (m.p. 144-146° C.).

R_{f}=0.24 (silica gel, 5:2:1 CH₂Cl₂:EtOAc:hexanes); ¹H NMR (400 MHz, CDCl₃) 8.72 (s, 1H), 7.98 (s, 1H), 7.83 (s, 1H), 7.81 (s, 1H), 7.26 (s, 1H), 7.23 (s, 1H), 5.38 (d, J=1.0 Hz, 2H)*, 5.36 (d, J=5.1 Hz, 2H)*, 5.32 (d, J=4.8 Hz, 2H)*, 3.56 (s, 3H), 3.54 (s, 3H), 3.54 (s, 3H), 2.66 (s, 3H), 2.46 (s, 3H); ¹³C NMR (125 MHz, CDCl₃) δ 201.6, 199.2, 174.7, 174.5, 154.2, 153.8, 153.1, 152.9 (2 signals), 152.3, 145.6, 145.1, 135.8, 134.1, 132.8, 127.4, 121.2, 120.6, 118.1, 115.8, 111.4, 110.6, 104.1, 103.8, 95.7, 95.6, 95.2, 95.1, 56.7, 56.5, (3 signals), 32.4, 28.9; IR (film, υ cm⁻¹) 1618, 1497, 1458, 1269, 1154; HRMS (ESI) calc. for C₃₄H₃₂NaO₁₄ [M+Na]⁺: 687.16840. obs. 687.16970. *Non-equivalent methylene protons.

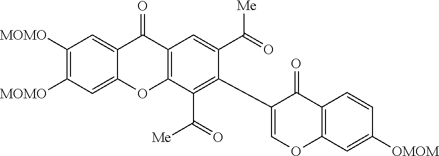

### 1,1′-(6,7-bis(methoxymethoxy)-3-(7-(methoxymethoxy)-4-oxo-4H-chromen-3-yl)-9-oxo-9H-xanthene-2,4-diyl)bis(ethan-1-one)

Following general procedure A for ynone dimerization, ynone (21) and ynone (22) were transformed into the protected vinaxanthone. The crude material was purified via silica gel column chromatography (5:2:1 CH₂Cl₂:EtOAc:hexanes) to give pure protected vinaxanthone (71 mg, 0.117 mmol, 46%) as a yellow solid (m.p. 210-212° C.). A side-product protected vinaxanthone (ynone (21) homodimer) (19 mg, 0.035 mmol, 27% with respect to ynone (21)) was also isolated.

R_{f}=0.35 (silica gel, 5:2:1 CH₂Cl₂:EtOAc:hexanes); ¹H NMR (400 MHz, CDCl₃) δ 8.72 (s, 1H), 8.28 (d, J=8.9 Hz, 1H), 7.82 (s, 1H), 7.81 (s, 1H), 7.26 (s, 1H), 7.11 (s, 1H), 7.08 (d, J=1.7 Hz, 1H), 5.37 (s, 2H), 5.31 (d, J=4.5 Hz, 2H)*, 3.54 (s, 3H), 3.53 (s, 3H), 3.52 (s, 3H), 2.65 (s, 3H), 2.46 (s, 3H); ¹³C NMR (125 MHz, CDCl₃) δ 201.5, 199.2, 174.8, 174.6, 163.2, 157.4, 153.9, 153.3, 152.9 (2 signals), 145.6, 136.0, 134.1, 133.1, 128.5, 127.4, 121.1 (2 signals), 118.2, 116.3, 115.3, 110.6, 104.1, 103.2, 95.6, 95.1, 94.4, 56.5 (3 signals), 32.4, 28.9; IR (film, υ cm⁻¹) 1642, 1621, 1456, 1262, 1155; HRMS (ESI) calc. for C₃₂H₂₈NaO₁₂ [M+Na]⁺: 627.14730. obs. 627.14770. *Non-equivalent methylene protons.

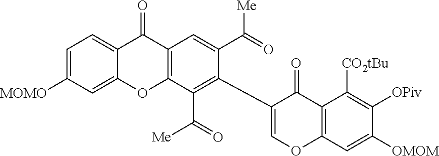

### tert-butyl 3-(2,4-diacetyl-6-(methoxymethoxy)-9-oxo-9H-xanthen-3-yl)-7-(methoxymethoxy)-4-oxo-6-(pivaloyloxy)-4H-chromene-5-carboxylate

Following general procedure A for ynone dimerization, ynone (22) and ynone (19) were transformed into the protected vinaxanthone. The crude material was purified via silica gel column chromatography (5:2:1 CH₂Cl₂:EtOAc:hexanes) to give pure protected vinaxanthone (117 mg, 0.157 mmol, 46%) as a pale yellow solid (m.p. 148-149° C.). A side-product protected vinaxanthone (ynone (19) homodimer) (72 mg, 0.076 mmol, 44% with respect to ynone (19)) and another side-product protected vinaxanthone (ynone (22) homodimer) (23 mg, 0.042 mmol, 25% with respect to ynone (22)) were also isolated.

R_{f}=0.51 (silica gel, 5:2:1 CH₂Cl₂:EtOAc:hexanes); ¹H NMR (400 MHz, CDCl₃) δ 8.68 (s, 1H), 8.13 (d, J=8.7 Hz, 1H), 7.82 (s, 1H), 7.19 (s, 1H), 7.11 (d, J=2.4 Hz, 1H), 7.08 (dd, J=8.7, 2.4 Hz, 1H), 5.29 (s, 2H), 5.27 (d, J=3.8 Hz, 2H)*, 3.51 (s, 3H), 3.47 (s, 3H), 2.64 (s, 3H), 2.45 (s, 3H), 1.69 (s, 9H), 1.39 (s, 9H); ¹³C NMR (125 MHz, CDCl₃) 201.2, 199.1, 175.5, 174.9, 173.3, 163.5, 161.9, 157.9, 154.7, 154.6, 154.1, 152.7, 136.3, 135.9, 133.9, 133.1, 129.0, 127.7, 127.4, 121.6, 120.7, 118.3, 115.9, 112.7, 103.7, 103.4, 94.8, 94.3, 83.3, 56.7, 56.4, 39.2, 32.4, 28.9, 28.2, 27.3; IR (film, υ cm⁻¹) 1615, 1463, 1252, 1156, 1091; HRMS (ESI) calc. for C₄₀H₄₀NaO₁₄ [M+Na]⁺: 767.23103. obs. 767.23034. *Non-equivalent methylene protons.

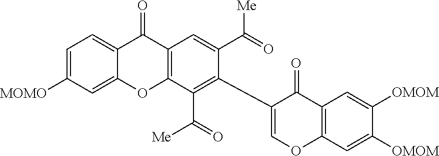

### 1,1′-(3-(6,7-bis(methoxymethoxy)-4-oxo-4H-chromen-3-yl)-6-(methoxymethoxy)-9-oxo-9H-xanthene-2,4-diyl)bis(ethan-1-one)

Following general procedure A for ynone dimerization, ynone (22) and ynone (21) were transformed into the protected vinaxanthone. The crude material was purified via silica gel column chromatography (5:2:1 CH₂Cl₂:EtOAc:hexanes) to give pure protected vinaxanthone (94 mg, 0.155 mmol, 45%) as a yellow solid (m.p. 84-85° C.). A side-product protected vinaxanthone (ynone (21) homodimer) (3 mg, 5.15 mol, 52% with respect to ynone (21)) and protected vinaxanthone (ynone (22) homodimer) (24 mg, 0.044 mmol, 3% with respect to ynone (22)) were also isolated.

R_{f}=0.33 (silica gel, 5:2:1 CH₂Cl₂:EtOAc:hexanes); ¹H NMR (400 MHz, CDCl₃) δ 8.73 (s, 1H), 8.13 (d, J=8.9 Hz, 1H), 7.99 (s, 1H), 7.81 (s, 1H), 7.23 (s, 1H), 7.11 (d, J=2.4 Hz, 1H), 7.08 (dd, J=8.9, 2.4, 1H), 5.38 (s, 2H), 5.35 (s, 2H), 5.28 (s, 2H), 3.56 (s, 3H), 3.57 (s, 3H), 3.51 (s, 3H), 2.66 (s, 3H), 2.46 (s, 3H); ¹³C NMR (125 MHz, CDCl₃) δ 201.6, 199.1, 175.1, 174.5, 161.9. 158.0, 154.2, 154.0, 153.2, 152.3, 145.1, 135.8, 134.1, 132.6, 127.8, 127.4, 121.7, 120.6, 118.4, 115.9 (2 signals), 111.4, 103.8, 103.4, 95.7, 95.2, 94.3, 56.7, 56.5, 56.5, 32.4, 28.9; IR (film, υ cm⁻¹) 1619, 1440, 1270, 1254, 1155; HRMS (ESI) calc. for C₃₂H₂₈NaO₁₂ [M+Na]⁺: 627.14730. obs. 627.14850.

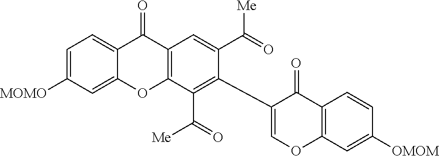

### 1,1′-(6-(methoxymethoxy)-3-(7-(methoxymethoxy)-4-oxo-4H-chromen-3-yl)-9-oxo-9H-xanthene-2,4-diyl)bis(ethan-1-one)

Following general procedure B for ynone dimerization, ynone (22) was transformed into the protected vinaxanthone. The crude material was purified via silica gel column chromatography (5:2:1 CH₂Cl₂:EtOAc:hexanes) to give pure protected vinaxanthone (12 mg, 0.022 mmol, 24%) as a pale yellow solid (m.p. 215-216° C.).

R_{f}=0.50 (silica gel, 5:2:1 CH₂Cl₂:EtOAc:hexanes); ¹H NMR (400 MHz, CDCl₃): 8.73 (s, 1H), 8.29 (d, J=9.2 Hz, 1H), 8.13 (d, J=8.9 Hz, 1H), 7.82 (s, 1H), 7.12 (d, J=2.1 Hz, 1H), 7.11 (dd, J=9.2, 2.1 Hz, 1H), 7.10 (d, J=2.4 Hz, 1H), 7.09 (dd, J=8.9, 2.4 Hz, 1H), 5.31 (s, 2H), 5.29 (s, 2H), 3.52 (s, 3H), 3.51 (s, 3H), 2.66 (s, 3H), 2.47 (s, 3H); ¹³C NMR (100 MHz, CDCl₃): δ 210.5, 199.1, 175.0, 174.7, 163.1, 161.9, 157.9, 157.3, 154.0, 153.2, 135.8, 134.1, 132.9, 128.4, 127.7, 127.4, 121.6, 121.0, 118.3, 116.2, 115.8, 115.3, 103.3, 103.2, 94.4, 94.3, 56.5, 56.4, 32.4, 28.9; IR (film, υ cm⁻¹): 1684, 1636, 1483, 1153; HRMS (ESI) calc. for C₃₀H₂₄NaO₁₀ [M+Na]⁺: 567.12617. obs. 567.12611.

I. General Procedure A for Protected Vinaxanthone Deprotection

To a stirred solution of protected vinaxanthone (1.0 equiv.) in CH₂Cl₂ at 0° C. was added a 1.0 M solution of boron trichloride in CH₂Cl₂ (2.0 equiv. per protecting group). The reaction mixture was stirred at 23° C. for 1 hour. The reaction mixture was then diluted with EtOAc and washed with brine (5×). The organic layer was dried over Na₂SO₄ and concentrated in vacuo to give crude vinaxanthone. Trituration with pentane:MeOH (ratio varies depending on substrate solubility) gave pure vinaxanthone.

J. General Procedure B for Protected Vinaxanthone Deprotection

A solution of protected vinaxanthone (1.0 equiv.) in 1.25 M methanolic HCl (10 equiv. per protected group) was stirred at 65° C. for 8 hrs. The reaction was followed by aliquot 1H NMR. The reaction mixture was then purged with N₂ and concentrated in vacuo to give crude vinaxanthone. Trituration with pentane:MeOH (ratio varies depending on substrate solubility) gave pure vinaxanthone.

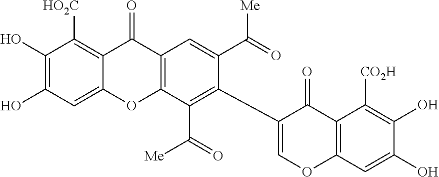 [SMILES: CC(=O)c1cc2c(=O)c3c(C(=O)O)c(O)c(O)cc3oc2c(C(C)=O)c1-c1coc2cc(O)c(O)c(C(=O)O)c2c1=O]

### 5,7-diacetyl-6-(5-carboxy-6,7-dihydroxy-4-oxo-4H-chromen-3-yl)-2,3-dihydroxy-9-oxo-9H-xanthene-1-carboxylic acid (vinaxanthone) (2)

Following general procedure A for protected vinaxanthone deprotection, protected vinaxanthone was transformed into pure vinaxanthone (2) (155 mg, 0.269 mmol, 98%), a yellow solid (m.p. 280° C. (decomp.)).

R_{f}=0.05 (silica gel, 20:1 EtOAc:AcOH); ¹H NMR (400 MHz, (CD₃)₂SO) δ 12.89 (bs, 1H), 12.72 (bs, 1H), 11.69 (bs, 1H), 11.44 (bs, 1H), 9.42 (bs, 2H), 8.53 (s, 1H), 8.18 (s, 1H), 6.96 (s, 1H), 6.94 (s, 1H), 2.55 (s, 3H), 2.53 (s, 3H); ¹³C NMR (125 MHz, (CD₃)₂SO) δ 201.1, 199.1, 172.9, 172.6, 167.4, 167.4, 154.1, 152.7, 152.5, 152.1, 150.7, 150.3, 141.7, 141.0, 136.2, 133.4, 132.6, 126.3, 120.8, 120.5, 119.8, 119.6, 112.4, 110.0, 102.4, 102.3, 32.1, 29.1; IR (KBr, υ cm⁻¹) 3236, 1683, 1653, 1472, 1288; HRMS (ESI) calc. for C₂₈H₁₅O₁₄ [M−H]⁻: 575.04673. obs. 575.04679.

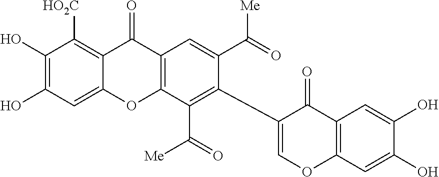 [SMILES: CC(=O)c1cc2c(=O)c3c(C(=O)O)c(O)c(O)cc3oc2c(C(C)=O)c1-c1coc2cc(O)c(O)cc2c1=O]

### 5,7-diacetyl-6-(6,7-dihydroxy-4-oxo-4H-chromen-3-yl)-2,3-dihydroxy-9-oxo-9H-xanthene-1-carboxylic acid (2a)

Following general procedure A for protected vinaxanthone deprotection, protected vinaxanthone was transformed into pure vinaxanthone (2a) (9 mg, 0.017 mmol, 97%), a tan solid (m.p. 248-250° C. (decomp.)).

R_{f}=0.14 (silica gel, 20:1 EtOAc:AcOH); ¹H NMR (400 MHz, (CD₃)₂SO) δ 12.73 (bs, 1H), 11.47 (bs, 1H), 10.87 (bs, 1H), 9.98 (bs, 1H), 9.44 (bs, 1), 8.57 (s, 1H), 8.17 (s, 1H), 7.48 (s, 1H), 6.96 (s, 1H), 6.95 (s, 1H), 2.54 (s, 3H), 2.50 (s, 3H); ¹³C NMR (125 MHz, (CD₃)₂SO) δ 201.2, 199.2, 173.4, 172.9, 167.4, 154.4, 152.7, 152.6, 152.5, 150.8, 150.7, 144.5, 144.7, 136.1, 133.6, 132.4, 126.3, 120.9, 119.8, 119.6, 113.5, 112.5, 108.7, 103.1, 102.3, 32.1, 29.1; IR (KBr, υ cm⁻¹) 3219, 1470, 1196, 803; HRMS (ESI) calc. for C₂₇H₁₅O₁₂ [M−H]⁻: 531.05690. obs. 531.05700.

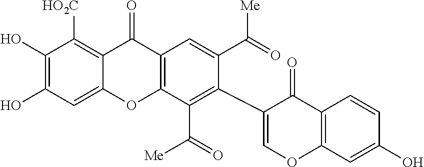 [SMILES: CC(=O)c1cc2c(=O)c3c(C(=O)O)c(O)c(O)cc3oc2c(C(C)=O)c1-c1coc2cc(O)ccc2c1=O]

### 5,7-diacetyl-2,3-dihydroxy-6-(7-hydroxy-4-oxo-4H-chromen-3-yl)-9-oxo-9H-xanthene-1-carboxylic acid (2b)

Following general procedure A for protected vinaxanthone deprotection, protected vinaxanthone was transformed into pure vinaxanthone (2b) (8 mg, 0.015 mmol, 96%), a tan solid (m.p. 254-255° C. (decomp.)).

R_{f}=0.31 (silica gel, 20:1 EtOAc:AcOH); ¹H NMR (400 MHz, (CD₃)₂SO) δ 12.70 (bs, 1H), 11.42 (bs, 1H), 11.15 (bs, 1H), 9.42 (bs, 1H), 8.57 (s, 1H), 8.17 (s, 1H), 8.10 (d, J=8.9 Hz, 1H), 6.98 (d, J=8.9 Hz, 1H), 6.96 (s, 1H), 6.92 (s, 1H), 2.56 (s, 3H), 2.54 (s, 3H); ¹³C NMR (125 MHz, (CD₃)₂SO) δ 201.1, 199.2, 173.6, 172.8, 167.3, 164.6, 157.2, 152.7, 152.6, 152.5, 150.7, 141.7, 136.5, 133.6, 132.8, 128.1, 126.2, 120.8, 120.3, 119.6, 114.9, 113.8, 112.4, 102.5, 102.3, 32.1, 29.2; IR (KBr, υ cm⁻¹) 3381, 1618, 1466, 1274; HRMS (ESI) calc. for C₂₇H₁₅O₁₁ [M−H]⁻: 515.06198. obs. 515.06245.

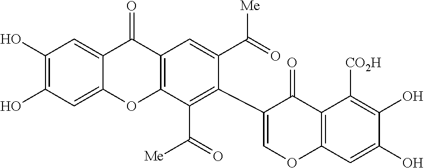 [SMILES: CC(=O)c1cc2c(=O)c3cc(O)c(O)cc3oc2c(C(C)=O)c1-c1coc2cc(O)c(O)c(C(=O)O)c2c1=O]

### 3-(2,4-diacetyl-6,7-dihydroxy-9-oxo-9H-xanthen-3-yl)-6,7-dihydroxy-4-oxo-4H-chromene-5-carboxylic acid (2c)

Following general procedure A for protected vinaxanthone deprotection, protected vinaxanthone was transformed into pure vinaxanthone (2c) (5 mg, 8.83 μmol, 89%), a tan solid (m.p. 225-226° C.).

R_{f}=0.13 (silica gel, 20:1 EtOAc:AcOH); ¹H NMR (400 MHz, (CD₃)₂SO) δ 11.71 (bs, 1H), 10.59 (bs, 1), 9.92 (bs, 1H), 9.44 (bs, 1H), 8.55 (s, 1H), 8.15 (s, 1H), 7.28 (s, 1H), 6.96 (s, 1H), 6.94 (s, 1H), 2.55 (s, 3H), 2.53 (s, 3H); ¹³C NMR (125 MHz, (CD₃)₂SO) δ 201.0, 199.0, 173.5, 172.6, 167.3, 154.0, 152.9, 152.8, 152.1, 151.0, 150.2, 144.9, 140.9, 135.9, 133.2, 132.9, 126.4, 120.6, 120.5, 119.7, 115.7, 110.0, 107.9, 102.9, 102.4, 32.2, 29.1; IR (KBr, υ cm⁻¹) 3393, 1624, 1577, 1466, 1290; HRMS (ESI) calc. for C₂₇H₁₅O₁₂ [M−H]⁻: 531.05690. obs. 531.05690.

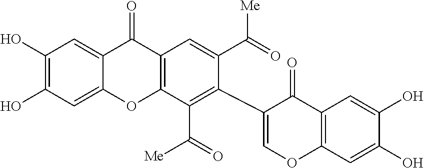 [SMILES: CC(=O)c1cc2c(=O)c3cc(O)c(O)cc3oc2c(C(C)=O)c1-c1coc2cc(O)c(O)cc2c1=O]

### 1,1′-(3-(6,7-dihydroxy-4-oxo-4H-chromen-3-yl)-6,7-dihydroxy-9-oxo-9H-xanthene-2,4-diyl)bis(ethan-1-one) (2d)

Following general procedure B for protected vinaxanthone deprotection, protected vinaxanthone was transformed into pure vinaxanthone (2d) (48 mg, 0.098 mmol, 97%), a magenta solid (m.p. 290° C. (decomp.)).

R_{f}=0.24 (silica gel, 20:1 EtOAc:AcOH); ¹H NMR (400 MHz, (CD₃)₂SO) δ 10.83 (bs, 1H), 10.55 (bs, 1H), 9.93 (bs, 2H), 8.58 (s, 1H), 8.12 (s, 1H), 7.49 (s, 1H), 7.28 (s, 1H), 6.94 (s, 1H), 6.93 (s, 1H), 2.55 (s, 3H), 2.53 (s, 3H); ¹³C NMR (125 MHz, (CD₃)₂SO) δ 201.1, 199.0, 173.6, 173.3, 154.3, 152.8 (2 signals), 152.4, 151.0, 150.6, 144.9, 144.5, 139.8, 135.8, 133.5, 132.7, 162.3, 120.7, 119.7, 115.7, 113.4, 108.6, 107.9, 102.9, 32.2, 29.1; IR (KBr, υ cm⁻¹) 3382, 1617, 1473, 1292; HRMS (ESI) calc. for C₂₆H₁₅O₁₀ [M−H]⁻: 487.06707. obs. 487.06709.

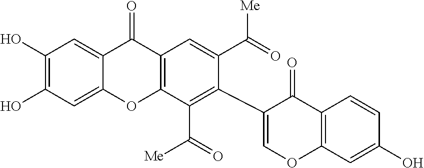 [SMILES: CC(=O)c1cc2c(=O)c3cc(O)c(O)cc3oc2c(C(C)=O)c1-c1coc2cc(O)ccc2c1=O]

### 1,1′-(6,7-dihydroxy-3-(7-hydroxy-4-oxo-4H-chromen-3-yl)-9-oxo-9H-xanthene-2,4-diyl)bis(ethan-1-one) (2e)

Following general procedure B for protected vinaxanthone deprotection, protected vinaxanthone was transformed into pure vinaxanthone (2e) (5 mg, 10.58 mol, 91%), a magenta solid (m.p. 218-220° C. (decomp.)).

R_{f}=0.09 (silica gel, 10:10:1 hexanes:EtOAc:AcOH); ¹H NMR (400 MHz, (CD₃)₂SO) δ 11.17 (bs, 1H), 10.59 (bs, 1H), 9.91 (bs, 1H), 8.58 (s, 1H), 8.14 (s, 1H), 8.09 (d, J=8.6 Hz, 1H), 7.29 (s, 1H), 6.98 (d, J=8.6 Hz, 1H), 6.94 (s, 1H), 6.91 (s, 1H), 2.55 (s, 3H), 2.54 (s, 3H); ¹³C NMR (125 MHz, (CD₃)₂SO) δ 201.0, 199.0, 173.5, 164.5, 157.2, 152.9, 152.8, 152.6, 151.1, 144.9, 136.2, 133.5, 133.2, 128.0, 126.3, 120.6, 120.3, 115.7, 114.9, 113.8, 107.9, 102.9, 102.5, 100.0, 32.2, 29.1; IR (KBr, υ cm⁻¹) 3406, 1617, 1560, 1466, 1273; HRMS (ESI) calc. for C₂₆H₁₅O₉ [M−H]⁻: 471.07216. obs. 471.07279.

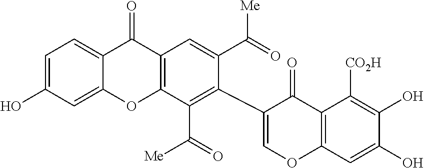 [SMILES: CC(=O)c1cc2c(=O)c3ccc(O)cc3oc2c(C(C)=O)c1-c1coc2cc(O)c(O)c(C(=O)O)c2c1=O]

### 3-(2,4-diacetyl-6-hydroxy-9-oxo-9H-xanthen-3-yl)-6,7-dihydroxy-4-oxo-4H-chromene-5-carboxylic acid (2f)

Following general procedure A for protected vinaxanthone deprotection, protected vinaxanthone was transformed into pure vinaxanthone (2f) (20 mg, 0.039 mmol, 96%), a yellow solid (m.p. 208-210° C. (decomp.)).

R_{f}=0.09 (silica gel, 20:1 EtOAc:AcOH); 1H NMR (400 MHz, (CD₃)₂SO) δ 11.72 (bs, 1H), 10.96 (bs, 1H), 9.42 (bs, 1H), 8.58 (s, 1H), 8.19 (s, 1H), 7.91 (d, J=8.9 Hz, 1H), 6.96 (dd, J=8.9, 2.4 Hz, 1H), 6.93 (s, 1H), 6.91 (d, J=2.4 Hz, 1H), 2.57 (s, 3H), 2.55 (s, 3H); ¹³C NMR (125 MHz, (CD₃)₂SO) δ 201.0, 198.9, 173.8, 172.5, 167.3, 163.0, 157.6, 154.0, 153.2, 152.2, 150.2, 140.9, 135.6, 133.3, 132.6, 127.2, 126.6, 121.6, 120.5, 119.8, 115.7, 115.4, 110.0, 102.4, 102.2, 32.2, 29.0; IR (KBr, a cm⁻¹) 3385, 1624, 1459, 1290, 1101; HRMS (ESI) calc. for C₂₇H₁₅O [M−H]⁻: 515.061989. obs. 515.06236.

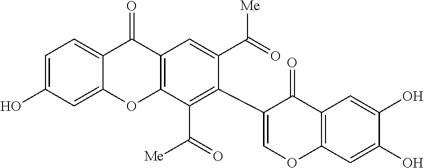 [SMILES: CC(=O)c1cc2c(=O)c3ccc(O)cc3oc2c(C(C)=O)c1-c1coc2cc(O)c(O)cc2c1=O]

### 1,1′-(3-(6,7-dihydroxy-4-oxo-4H-chromen-3-yl)-6-hydroxy-9-oxo-9H-xanthene-2,4-diyl)bis(ethan-1-one) (2g)

Following general procedure B for protected vinaxanthone deprotection, protected vinaxanthone was transformed into pure vinaxanthone (2g) (13 mg, 0.028 mmol, 98%), a magenta solid (m.p. 208-210° C. (decomp.)).

R_{f}=0.06 (silica gel, 10:10:1 hexanes:EtOAc:AcOH); ¹H NMR (400 MHz, (CD₃)₂SO) δ 10.97 bs, 1H), 10.86 (bs, 1H), 9.98 (bs, 1H), 8.62 (s, 1H), 8.19 (s, 1H), 7.91 (d, J=8.9 Hz, 1H), 7.48 (s, 1H), 6.96 (d, J=9.2 Hz, 1H), 6.95 (s, 1H), 6.91 (s, 1H), 2.57 (s, 3H), 2.55 (s, 3H); ¹³C NMR (125 MHz, (CD₃)₂SO) δ 201.2, 199.0, 173.9, 173.3, 163.1, 157.7, 154.4, 153.2, 152.5, 150.7, 144.5, 135.6, 133.6, 132.4, 127.3, 126.6, 121.7, 119.9, 115.8, 115.4, 113.5, 108.7, 103.1, 102.3, 32.3, 29.1; IR (KBr, υ cm⁻¹) 3299, 1624, 1470, 1295; HRMS (ESI) calc. for C₂₆H₁₅O₉ [M−H]⁻: 471.07216. obs. 471.07231.

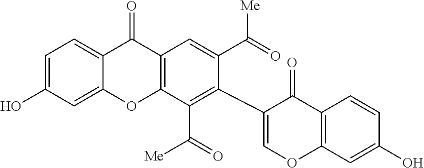 [SMILES: CC(=O)c1cc2c(=O)c3ccc(O)cc3oc2c(C(C)=O)c1-c1coc2cc(O)ccc2c1=O]

### 1,1′-(6-hydroxy-3-(7-hydroxy-4-oxo-4H-chromen-3-yl)-9-oxo-9H-xanthene-2,4-diyl)bis(ethan-1-one) (2h)

Following general procedure B for protected vinaxanthone deprotection, protected vinaxanthone was transformed into pure vinaxanthone (2h) (8 mg, 0.018 mmol, 99%), a tan solid (m.p. 340° C. (decomp.)).

R_{f}=0.16 (silica gel, 10:10:1 hexanes:EtOAc:AcOH); ¹H NMR (500 MHz, (CD₃)₂SO): δ 11.14 (s, 1H), 10.93 (s, 1H), 8.61 (s, 1H), 8.19 (s, 1H), 8.10 (d, J=7.2 Hz, 1H), 7.91 (d, J=7.2 Hz, 1H), 6.91 (s, 1H), 6.91 (s, 1H), 6.98 (ddd, J=11, 7.2, 2.0 Hz, 2H), 2.57 (s, 3H), 2.57 (s, 3H); ¹³C NMR (125 MHz, (CD₃)₂SO): δ 201.0, 199.0, 173.9, 173.6, 164.5, 163.1, 157.7, 157.2, 153.3, 152.8, 136.0, 133.6, 132.9, 128.1, 127.3, 126.6, 121.6, 120.5, 115.8, 115.5, 114.9, 113.8, 102.5, 102.3, 32.3, 29.1; IR (KBr, υ cm⁻¹): 3351, 1619, 1468, 1002; HRMS (ESI) calc. for C₂₆H₁₆NaO₈ [M+Na]: 479.07374. obs. 479.07433.

All of the compounds, formulations, and methods disclosed and claimed herein can be made and executed without undue experimentation in light of the present disclosure. While the compounds, formulations, and methods of this invention have been described in terms of preferred embodiments, it will be apparent to those of skill in the art that variations may be applied to the compounds, formulations, and methods, as well as in the steps or in the sequence of steps of the method described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references to the extent that they provide exemplary procedural or other details supplementary to those set forth herein, are specifically incorporated herein by reference.
Axelrod, et al., Angew. Chem. Int. Ed., 52(12):3421-3424, 2013.
Baier Leach, et al., Biotechnol. Bioeng., 82:578-589, 2003.
Gros, et al., Biomaterials, 31:6719-6729, 2010.
Handbook of Pharmaceutical Salts: Properties, and Use, Stahl and Wermuth Eds.), Verlag Helvetica Chimica Acta, 2002.
Kaneko, et al., Nature Medicine, 12:1380-1389, 2006.
Madigan, et al., Respir. Physiol. Neurobiol., 169:183-199, 2009.
March's Advanced Organic Chemistry: Reactions, Mechanisms, and Structure, 2007.
Omoto, et al., PLoS One, 7:e47716, 2012
Struve, et al., Glia, 52:16-24, 2005.
Tatsuta, et al., Chem. Lett., 30:10-11, 2007.
Wilson and Danishefsky, Acc. Chem. Res., 39:539-549, 2006.

1. A method of preparing a compound of the formula: 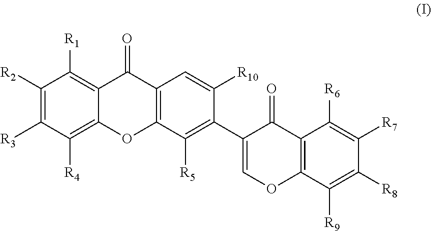
wherein:
R₁, R₂, R₃, R₄, R₆, R₇, R₈, and R₉ are each independently hydrogen, amino, carboxy, halo, hydroxy, mercapto, or
alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅,
wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and
R₅ and R₁₀ are each independently hydrogen, acyl_{(C≦12)}, or substituted acyl_{(C≦12)};
or a salt thereof;
comprising reacting in a reaction mixture a compound of the formula: 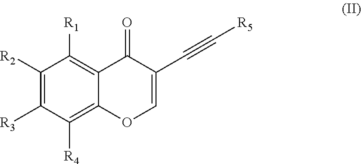
wherein:
R₁, R₂, R₃, and R₄ are each independently hydrogen, amino, carboxy, halo, hydroxy, mercapto, or
alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅,
wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and
R₅ is hydrogen, acyl_{(C≦12)}, or substituted acyl_{(C≦12)};
with water in a first solvent.

2. The method of claim 1, wherein the compound of formula I is further defined as: 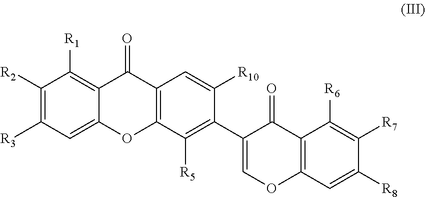
wherein:
R₁, R₂, R₃, R₆, R₇, and R₈ are each independently hydrogen, amino, carboxy, halo, hydroxy, mercapto, or
alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅,
wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and
R₅ and R₁₀ are each independently hydrogen, acyl_{(C≦12)}, or substituted acyl_{(C≦12)};
or a salt thereof.

3. The method of claim 1, wherein the compound of formula I is further defined as: 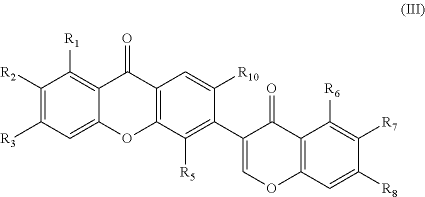
wherein:
R₁, R₂, R₃, R₆, R₇, and R₈ are each independently hydrogen, carboxy, hydroxy, or
alkoxy_{(C≦12)}, acyl_{(C≦12)}, substituted alkoxy_{(C≦12)}, substituted acyl_{(C≦12)}, —OX₁, or —C(O)OX₅,
wherein: X₁ is a hydroxy protecting group and X₅ is a carboxy protecting group; and
R₅ and R₁₀ are each independently hydrogen, acyl_{(C≦12)}, or substituted acyl_{(C≦12)};
or a salt thereof.

4. The method of claim 1, wherein the compound of formula I is further defined as: 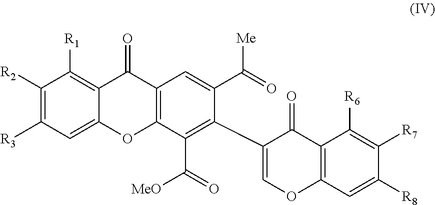
wherein:
R₁, R₂, R₃, R₆, R₇, and R₈ are each independently hydrogen, carboxy, hydroxy, or
alkoxy_{(C≦12)}, acyl_{(C≦12)}, substituted alkoxy_{(C≦12)}, substituted acyl_{(C≦12)}, —OX₁, or —C(O)OX₅,
wherein: X₁ is a hydroxy protecting group and X₅ is a carboxy protecting group;
or a salt thereof.

5. The method of claim 1, wherein R₁ is hydrogen.

6. The method of claim 1, wherein R₁ is carboxy.

7. The method of claim 1, wherein R₁ is hydroxy.

8. The method of claim 1, wherein R₁ is —OX₁.

9. The method of claim 8, wherein X₁ is pivaloyl or methoxymethyl.

10. The method of claim 1, wherein R₁ is —C(O)OX₅.

11. The method of claim 10, wherein X₅ is t-butyl.

12. The method of claim 1, wherein R₂ is hydrogen.

13. The method of claim 1, wherein R₂ is carboxy.

14. The method of claim 1, wherein R₂ is hydroxy.

15. The method of claim 1, wherein R₂ is —OX₁.

16. The method of claim 15, wherein X₁ is pivaloyl or methoxymethyl.

17. The method of claim 1, wherein R₂ is —C(O)OX₅.

18. The method of claim 17, wherein X₅ is t-butyl.

19. The method of claim 1, wherein R₃ is hydrogen.

20. The method of claim 1, wherein R₃ is carboxy.

21. The method of claim 1, wherein R₃ is hydroxy.

22. The method of claim 1, wherein R₃ is —OX₁.

23. The method of claim 22, wherein X₁ is pivaloyl or methoxymethyl.

24. The method of claim 1, wherein R₃ is —C(O)OX₅.

25. The method of claim 24, wherein X₅ is t-butyl.

26. The method of claim 1, wherein R₆ is hydrogen.

27. The method of claim 1, wherein R₆ is carboxy.

28. The method of claim 1, wherein R₆ is hydroxy.

29. The method of claim 1, wherein R₆ is —OX₁.

30. The method of claim 29, wherein X₁ is pivaloyl or methoxymethyl.

31. The method of claim 1, wherein R₆ is —C(O)OX₅.

32. The method of claim 31, wherein X₅ is t-butyl.

33. The method of claim 1, wherein R₇ is hydrogen.

34. The method of claim 1, wherein R₇ is carboxy.

35. The method of claim 1, wherein R₇ is hydroxy.

36. The method of claim 1, wherein R₇ is —OX₁.

37. The method of claim 36, wherein X₁ is pivaloyl or methoxymethyl.

38. The method of claim 1, wherein R₇ is —C(O)OX₅.

39. The method of claim 38, wherein X₅ is t-butyl.

40. The method of claim 1, wherein R₈ is hydrogen.

41. The method of claim 1, wherein R₈ is carboxy.

42. The method of claim 1, wherein R₈ is hydroxy.

43. The method of claim 1, wherein R₈ is —OX₁.

44. The method of claim 43, wherein X₁ is pivaloyl or methoxymethyl.

45. The method of claim 1, wherein R₈ is —C(O)OX₅.

46. The method of claim 45, wherein X₅ is methyl or t-butyl.

47. The method of claim 1, wherein R₄ is hydrogen.

48. The method of claim 1, wherein R₉ is hydrogen.

49. The method of claim 1, wherein R₅ is acyl_{(C≦12)} or substituted acyl_{(C≦12)}.

50. The method of claim 49, wherein R₅ is —C(O)Me.

51. The method of claim 1, wherein R₁₀ is acyl_{(C≦12)} or substituted acyl_{(C≦12)}.

52. The method of claim 51, wherein R₁₀ is —C(O)Me or —C(O)OMe.

53. The method of claim 1, wherein R₁₁ is acyl_{(C≦12)} or substituted acyl_{(C≦12)}.

54. The method of claim 53, wherein R₁₁ is —C(O)Me or —C(O)OMe.

55. The method of claim 1, wherein R₁₂ is acyl_{(C≦12)} or substituted acyl_{(C≦12)}.

56. The method of claim 55, wherein R₁₂ is —C(O)Me or —C(O)OMe.

57. The method of claim 1, wherein the compound of formula I is further defined as: 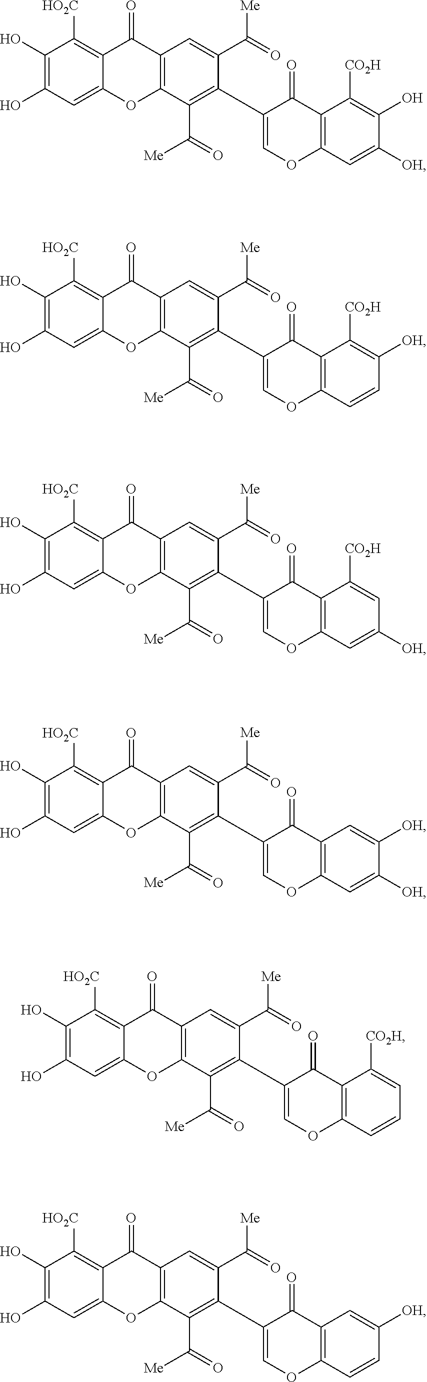 [SMILES: CC(=O)c1cc2c(=O)c3c(C(=O)O)c(O)c(O)cc3oc2c(C(C)=O)c1-c1coc2cc(O)c(O)c(C(=O)O)c2c1=O ; CC(=O)c1cc2c(=O)c3c(C(=O)O)c(O)c(O)cc3oc2c(C(C)=O)c1-c1coc2ccc(O)c(C(=O)O)c2c1=O ; CC(=O)c1cc2c(=O)c3c(C(=O)O)c(O)c(O)cc3oc2c(C(C)=O)c1-c1coc2cc(O)cc(C(=O)O)c2c1=O ; CC(=O)c1cc2c(=O)c3c(C(=O)O)c(O)c(O)cc3oc2c(C(C)=O)c1-c1coc2cc(O)c(O)cc2c1=O ; CC(=O)c1cc2c(=O)c3c(C(=O)O)c(O)c(O)cc3oc2c(C(C)=O)c1-c1coc2cccc(C(=O)O)c2c1=O ; CC(=O)c1cc2c(=O)c3c(C(=O)O)c(O)c(O)cc3oc2c(C(C)=O)c1-c1coc2ccc(O)cc2c1=O] 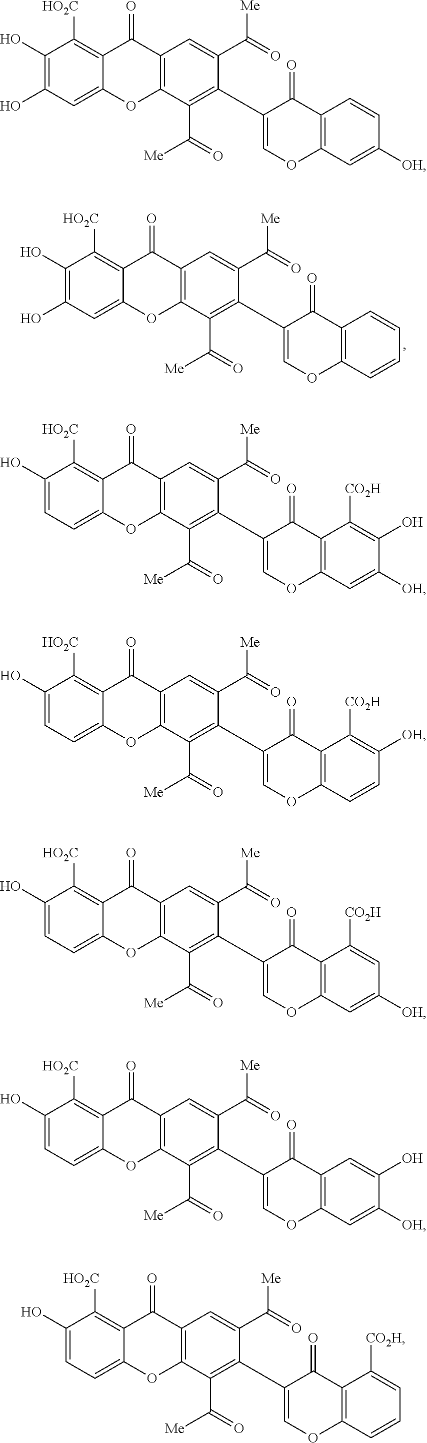 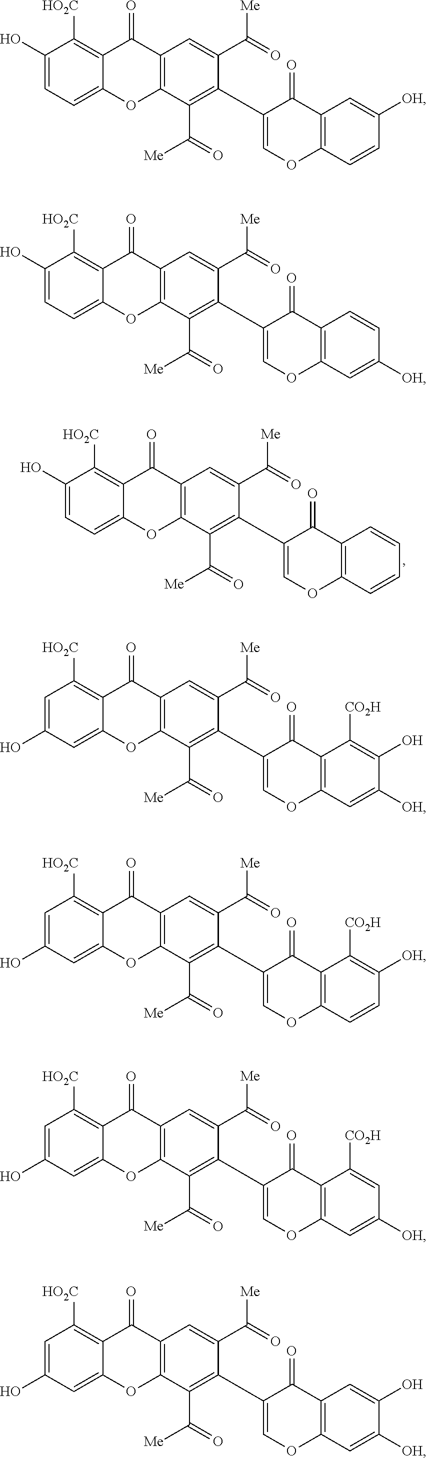 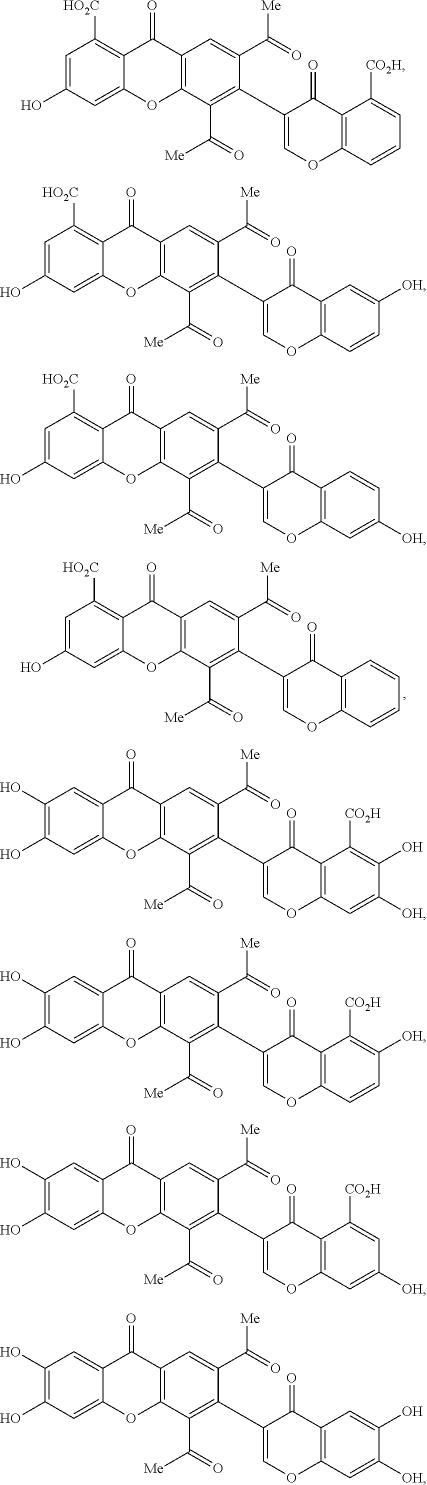 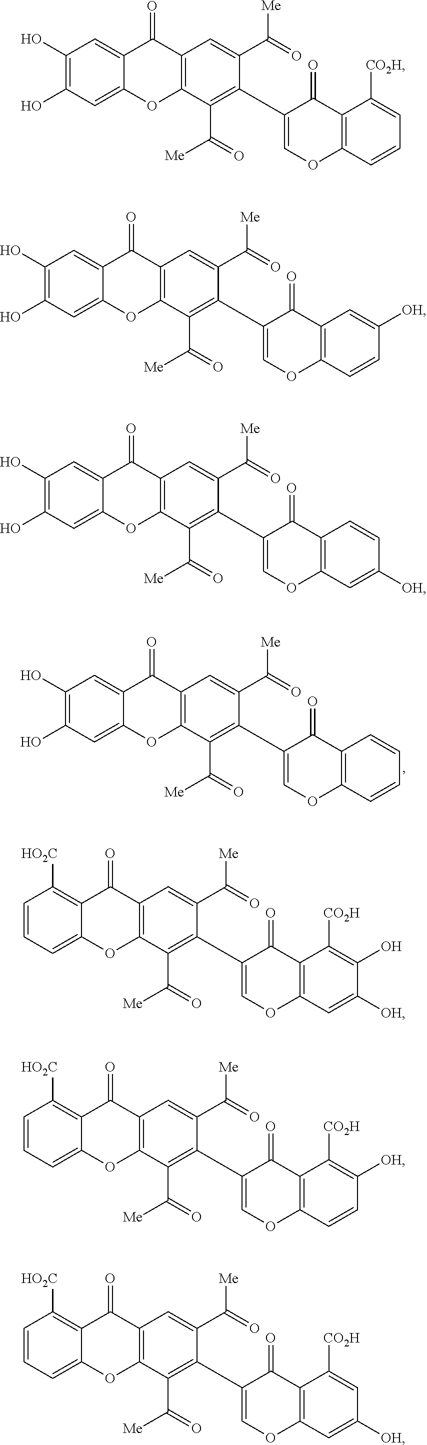 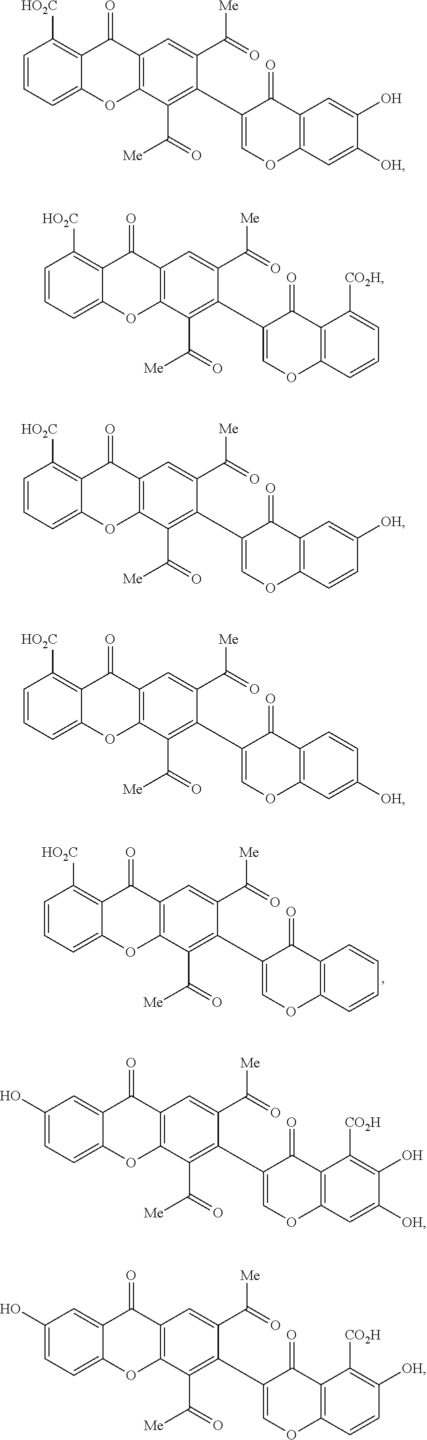 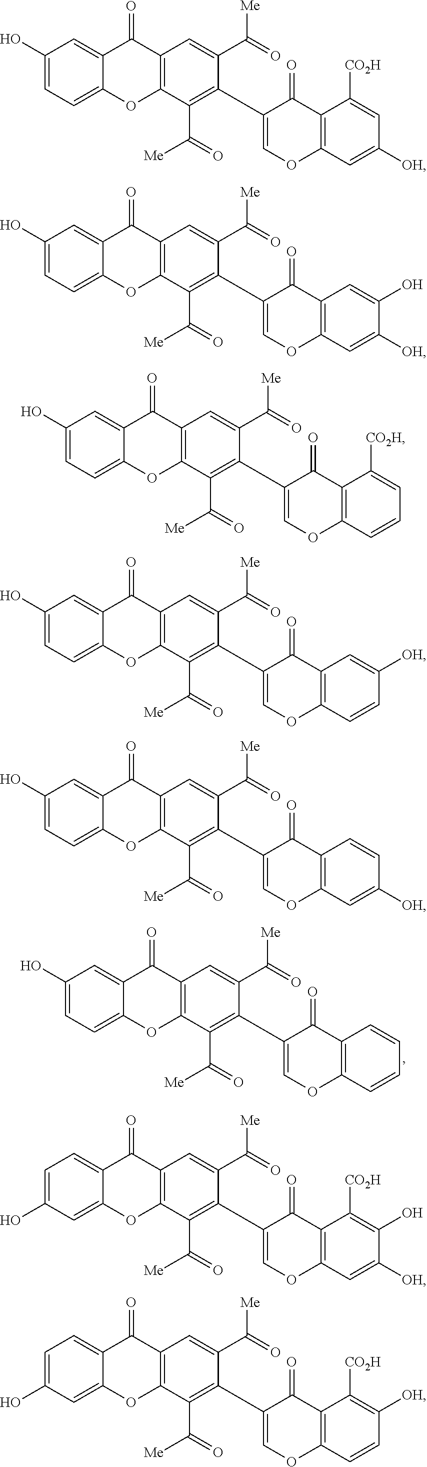 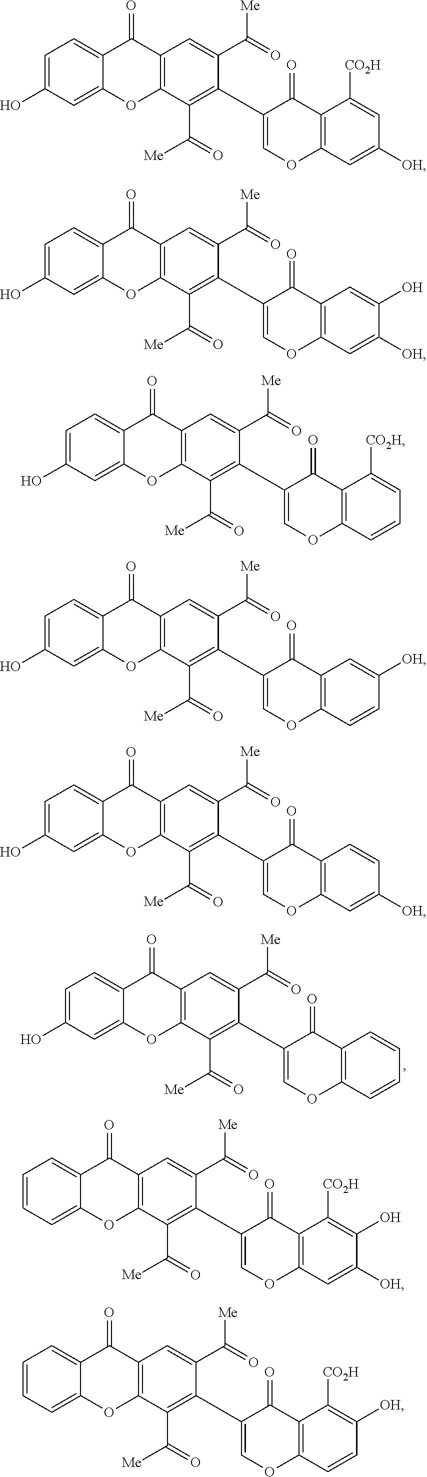 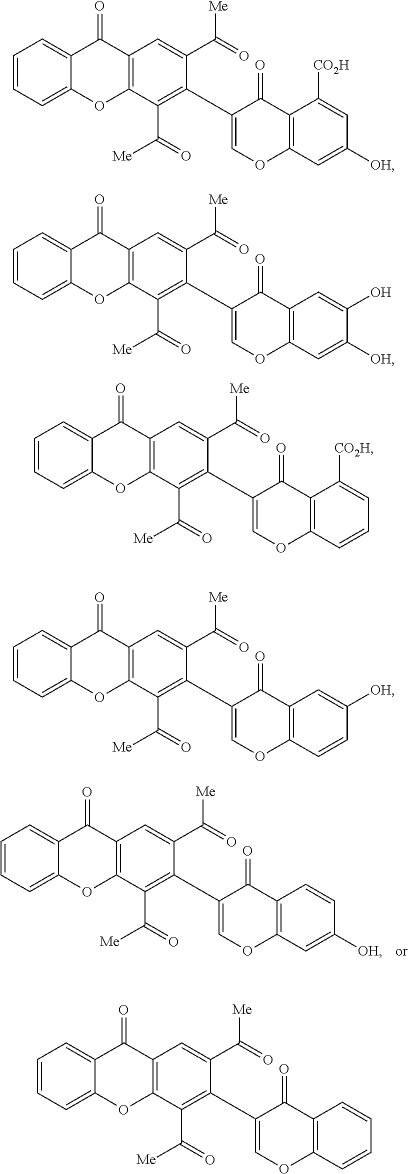
or a salt or tautomer thereof.

58. The method of claim 1, wherein the reaction further comprises a first base.

59. The method of claim 58, wherein the first base is a nitrogenous base.

60. The method of claim 58, wherein the first base is an tertiary amine_{(C≦18)}.

61. The method of claim 58, wherein the first base is a trialkylamine_{(C≦18)}.

62. The method of claim 58, wherein the first base is triethylamine.

63. The method of claim 1, wherein the first solvent is an organic solvent.

64. The method of claim 63, wherein the first solvent is a substituted alkane_{(C≦8)} or amide_{(C≦8)}.

65. The method of claim 63, wherein the first solvent is acetonitrile.

66. The method of claim 1, wherein the reaction comprises adding from about 0.01 equivalents to about 5.0 equivalents of water relative to the compound of formula II.

67. The method of claim 66, wherein the reaction comprises adding from about 0.1 equivalents to about 3.0 equivalents of water.

68. The method of claim 66, wherein the reaction comprises adding about 0.5 equivalents of water.

69. The method of claim 1, wherein the reaction comprises adding from about 1 equivalent to about 20.0 equivalents of the first base relative to the compound of formula II.

70. The method of claim 69, wherein the reaction comprises adding from about 5.0 equivalents to about 15.0 equivalents of the first base.

71. The method of claim 69, wherein the reaction comprises adding about 10 equivalents of the first base.

72. The method of claim 1, wherein the reaction comprises performing the reaction at a first temperature from about 0° C. to about 80° C.

73. The method of claim 72, wherein the first temperature is from about 0° C. to about 40° C.

74. The method of claim 72, wherein the first temperature is about 23° C.

75. The method of claim 73, wherein the first temperature is about room temperature.

76. The method of claim 1, wherein the reaction comprises performing the reaction for a first time period from about 10 minutes to about 36 hours.

77. The method of claim 76, wherein the first time period is about 10 hours to about 24 hours.

78. The method of claim 76, wherein the first time period is about 16 hours.

79. The method of claim 1, wherein the reaction further comprises mixing the reaction mixture.

80. The method of claim 1, wherein the reaction comprises adding from about 100 equivalents to about 2500 equivalents of water relative to the compound of formula II.

81. The method of claim 80, wherein the reaction comprises adding from about 500 equivalents to about 1500 equivalents of water.

82. The method of claim 80, wherein the reaction comprises adding about 1000 equivalents of water.

83. The method of claim 1, wherein the reaction comprises performing the reaction for a first time period from about 10 minutes to about 6 hours.

84. The method of claim 83, wherein the first time period is about 30 minutes to about 4 hours.

85. The method of claim 83, wherein the first time period is about 1 hour.

86. The method of claim 1, wherein the method further comprises removing the solvent in vacao.

87. The method of claim 1, wherein the method further comprises drying the reaction using sodium sulfate.

88. The method of claim 1, wherein the method further comprises adding after a first time period a compound of the formula: 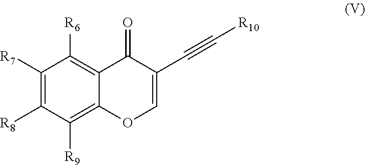
wherein:
R₆, R₇, R₈, and R₉ are each independently hydrogen, amino, carboxy, halo, hydroxy, mercapto, or
alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅,
wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and
R₁₀ is hydrogen, acyl_{(C≦12)}, or substituted acyl_{(C≦12)};
to a second solvent and reacting for a second time period.

89. The method of claim 88, wherein the method further comprises adding a second base.

90. The method of claim 89, wherein the base is a nitrogenous base.

91. The method of claim 89, wherein the base is an tertiary amine_{(C≦18)}.

92. The method of claim 89, wherein the base is a trialkylamine_{(C≦18)}.

93. The method of claim 89, wherein the base is triethylamine.

94. The method of claim 88, wherein the reaction comprises adding from about 0.1 equivalents to about 3.0 equivalents of the compound of formula V relative to the compound of formula II.

95. The method of claim 94, wherein the reaction comprises adding from about 0.5 equivalents to about 2.0 equivalents of the compound of formula V.

96. The method of claim 94, wherein the reaction comprises adding about 1.0 equivalents of the compound of formula V.

97. The method of claim 88, wherein the reaction comprises adding from about 0.1 equivalents to about 3.0 equivalents of the second base relative to the compound of formula II.

98. The method of claim 97, wherein the reaction comprises adding from about 0.5 equivalents to about 2.0 equivalents of the second base.

99. The method of claim 97, wherein the reaction comprises adding about 1.0 equivalents of the second base.

100. The method of claim 88, wherein the second solvent is an organic solvent.

101. The method of claim 100, wherein the second solvent is a substituted alkane_{(C≦8)} or amide_{(C≦8)}.

102. The method of claim 100, wherein the second solvent is acetonitrile.

103. The method of claim 88, wherein the reaction comprises performing the reaction at a second temperature from about 0° C. to about 80° C.

104. The method of claim 103, wherein the second temperature is from about 0° C. to about 40° C.

105. The method of claim 103, wherein the second temperature is about 23° C.

106. The method of claim 104, wherein the second temperature is about room temperature.

107. The method of claim 88, wherein the reaction comprises performing the reaction for a second time period from about 10 minutes to about 36 hours.

108. The method of claim 107, wherein the second time period is about 10 hours to about 24 hours.

109. The method of claim 107, wherein the second time period is about 16 hours.

110. The method of claim 88, wherein the reaction further comprises mixing the compound of formula II, the compound of formula V, and the second base in the second solvent.

111. The method of claim 1, wherein the reaction has a yield of greater than 25%.

112. The method of claim 111, wherein the yield is greater than 50%.

113. The method of claim 111, wherein the yield is greater than 70%.

114. A method of preparing a compound of the formula: 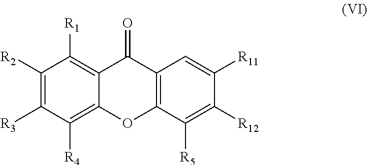
wherein:
R₁, R₂, R₃, and R₄ are each independently hydrogen, amino, carboxy, halo, hydroxy, mercapto, or
alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅,
wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group;
R₅ is hydrogen, acyl_{(C≦12)}, or substituted acyl_{(C≦12)}; and
R₁₁ and R₁₂ are each independently alkyl_{(C≦12)}, aryl_{(C≦12)}, acyl_{(C≦12)}, or a substituted version of any of these groups;
or a salt thereof;
comprising reacting a compound of the formula: 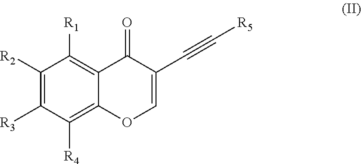
wherein:
R₁, R₂, R₃, and R₄ are each independently hydrogen, amino, carboxy, halo, hydroxy, mercapto, or
alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅,
wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and
R₅ is hydrogen, acyl_{(C≦12)}, or substituted acyl_{(C≦12)};
with a compound of the formula: 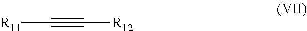
wherein:
R₁₁ and R₁₂ are each independently alkyl_{(C≦12)}, aryl_{(C≦12)}, acyl_{(C≦12)}, or a substituted version of any of these groups;
in the presence of a base and water in a solvent.

115. A method of preparing a compound of the formula: 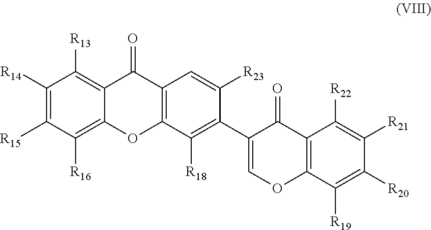
wherein:
R₁₃, R₁₄, R₁₅, R₁₆, R₁₉, R₂₀, R₂₁, and R₂₂ are each independently hydrogen, amino, carboxy, halo, hydroxy, mercapto, or
alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅,
wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and
R₁₈ and R₂₃ are each independently acyl_{(C≦18)} or substituted acyl_{(C≦18)};
or a salt thereof;
comprising
A) reacting a compound of the formula: 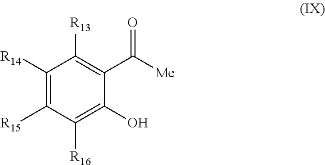
wherein: R₁₃, R₁₄, R₁₅, and R₁₆ are as defined above; with Me₂NCH(OMe)₂ in the presence of a solvent to form a compound of the formula: 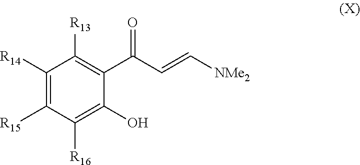
wherein: R₁₃, R₁₄, R₁₅, and R₁₆ are as defined above;
B) reacting the compound of formula X with iodide in a solvent to form a compound of the formula: 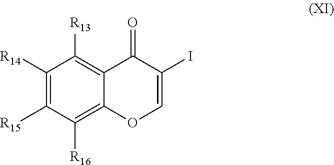
wherein: R₁₃, R₁₄, R₁₅, and R₁₆ are as defined above;
C) reacting the compound of formula X with a compound of the formula: 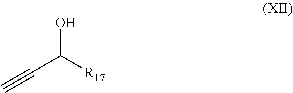
wherein:
R₁₇ is hydrogen, alkyl_{(C≦17)}, cycloalkyl_{(C≦17)}, alkenyl_{(C≦17)}, alkynyl_{(C≦17)}, aryl_{(C≦17)}, aralkyl_{(C≦17)}, heteroaryl_{(C≦17)}, heteroaralkyl_{(C≦17)}, heterocycloalkyl_{(C≦17)}, or a substituted version of any of these groups;
in the presence of a transition metal catalyst and a base in a solvent to form a compound of the formula: 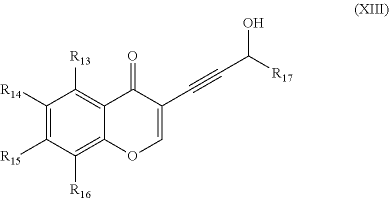
wherein: R₁₃, R₁₄, R₁₅, R₁₆, and R₁₇ are as defined above;
D) reacting the compound of formula XIII with an oxidizing agent in a solvent to form a compound of the formula: 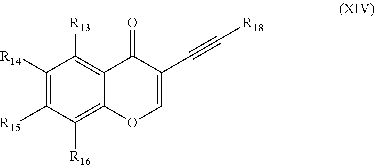
wherein:
R₁₃, R₁₄, R₁₅, and R₁₆ are as defined above; and
R₁₈ is acyl_{(C≦18)} or substituted acyl_{(C≦18)}; and
E) reacting the compound of formula XIV with a base and water in a solvent to form the compound of formula VIII wherein: R₁₃ and R₁₉, R₁₄ and R₂₀, R₁₅ and R₂₁, R₁₆ and R₂₂, and R₁₈ and R₂₃ are the same and as defined above; or
F) reacting the compound of formula XIV with a compound of the formula: 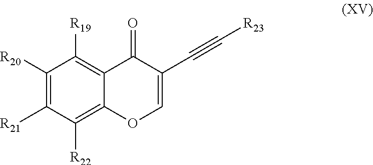
wherein: R₁₉, R₂₀, R₂₁, R₂₂, and R₂₃ are as defined above; in the presence of a base and water in a solvent to form the compound of formula VIII.

116. The method of claim 115, wherein R₁₃ is hydrogen, carboxy, hydroxy, or alkyl_{(C≦12)}, acyl_{(C≦12)}, or a substituted version of any of these groups, or —OX₁ or —C(O)OX₅, wherein: X₁ is a hydroxy protecting group and X₅ is a carboxy protecting group.

117. The method of claim 115, wherein R₁₄ is hydrogen, carboxy, hydroxy, or alkyl_{(C≦12)}, acyl_{(C≦12)}, or a substituted version of any of these groups, or —OX₁ or —C(O)OX₅, wherein: X₁ is a hydroxy protecting group and X₅ is a carboxy protecting group.

118. The method of claim 115, wherein R₁₅ is hydrogen, carboxy, hydroxy, or alkyl_{(C≦12)}, acyl_{(C≦12)}, or a substituted version of any of these groups, or —OX₁ or —C(O)OX₅, wherein: X₁ is a hydroxy protecting group and X₅ is a carboxy protecting group.

119. The method of claim 115, wherein R₁₆ is hydrogen, carboxy, hydroxy, or alkyl_{(C≦12)}, acyl_{(C≦12)}, or a substituted version of any of these groups, or —OX₁ or —C(O)OX₅, wherein: X₁ is a hydroxy protecting group and X₅ is a carboxy protecting group.

120. The method of claim 115, wherein R₁₉ is hydrogen, carboxy, hydroxy, or alkyl_{(C≦12)}, acyl_{(C≦12)}, or a substituted version of any of these groups, or —OX₁ or —C(O)OX₅, wherein: X₁ is a hydroxy protecting group and X₅ is a carboxy protecting group.

121. The method of claim 115, wherein R₂₀ is hydrogen, carboxy, hydroxy, or alkyl_{(C≦12)}, acyl_{(C≦12)}, or a substituted version of any of these groups, or —OX₁ or —C(O)OX₅, wherein: X₁ is a hydroxy protecting group and X₅ is a carboxy protecting group.

122. The method of claim 115, wherein R₂₁ is hydrogen, carboxy, hydroxy, or alkyl_{(C≦12)}, acyl_{(C≦12)}, or a substituted version of any of these groups, or —OX₁ or —C(O)OX₅, wherein: X₁ is a hydroxy protecting group and X₅ is a carboxy protecting group.

123. The method of claim 115, wherein R₂₂ is hydrogen, carboxy, hydroxy, alkyl_{(C≦12)}, acyl_{(C≦12)}, substituted alkyl_{(C≦12)}, substituted acyl_{(C≦12)}, or —OX₁ or —C(O)OX₅, wherein:
X₁ is a hydroxy protecting group and X₅ is a carboxy protecting group.

124. The method of claim 115, wherein R₁₇ is hydrogen, alkyl_{(C≦17)}, cycloalkyl_{(C≦17)}, aryl_{(C≦17)}, aralkyl_{(C≦17)}, heteroaryl_{(C≦17)}, heteroaralkyl_{(C≦17)}, or a substituted version of any of these groups.

125. The method of claim 124, wherein R₁₇ is hydrogen, alkyl_{(C≦17)}, cycloalkyl_{(C≦17)}, aryl_{(C≦17)}, substituted alkyl_{(C≦17)}, substituted cycloalkyl_{(C≦17)}, or substituted aryl_{(C≦17)}.

126. The method of claim 115, wherein the reaction of step A) comprises adding from about 1.0 equivalents to about 10.0 equivalents of Me₂NCH(OMe)₂ relative to the compound of formula IX.

127. The method of claim 126, wherein the reaction of step A) comprises adding from about 2.0 equivalents to about 8.0 equivalents of Me₂NCH(OMe)₂.

128. The method of claim 126, wherein the reaction of step A) comprises adding about 5.0 equivalents of Me₂NCH(OMe)₂.

129. The method of claim 115, wherein the solvent of step A) is a substituted alkane_{(C≦8)}.

130. The method of claim 115, wherein the solvent of step A) is dimethoxyethane.

131. The method of claim 115, wherein the reaction of step A) comprises performing the reaction at a temperature from about 50° C. to about 120° C.

132. The method of claim 131, wherein the temperature is from about 60° C. to about 100° C.

133. The method of claim 131, wherein the temperature is about 85° C.

134. The method of claim 115, wherein the reaction of step A) comprises performing the reaction for a time period from about 1 hour to about 12 hours.

135. The method of claim 134, wherein the time period is about 2 hours to about 6 hours.

136. The method of claim 134, wherein the time period is about 4 hours.

137. The method of claim 115, wherein the reaction of step A) further comprises mixing the compound of formula IX and Me₂NCH(OMe)₂ in the solvent.

138. The method of claim 115, wherein the reaction of step B) comprises adding from about 0.5 equivalents to about 5.0 equivalents of I₂ relative to the compound of formula X.

139. The method of claim 138, wherein the reaction of step B) comprises adding from about 1.0 equivalent to about 3.0 equivalents of I₂.

140. The method of claim 138, wherein the reaction of step B) comprises adding about 2.0 equivalents of I₂.

141. The method of claim 115, wherein the solvent of step B) is a substituted alkane_{(C≦8)}.

142. The method of claim 115, wherein the solvent of step B) is chloroform.

143. The method of claim 115, wherein the reaction of step B) comprises performing the reaction at a temperature from about 0° C. to about 50° C.

144. The method of claim 143, wherein the temperature is from about 15° C. to about 30° C.

145. The method of claim 143, wherein the temperature is about 23° C.

146. The method of claim 143, wherein the temperature is room temperature.

147. The method of claim 115, wherein the reaction of step B) comprises performing the reaction for a time period from about 15 minutes to about 4 hours.

148. The method of claim 147, wherein the time period is about 30 minutes to about 2 hours.

149. The method of claim 147, wherein the time period is about 1 hour.

150. The method of claim 115, wherein the reaction of step B) further comprises mixing the compound of formula X and I₂ in the solvent.

151. The method of claim 115, wherein the transition metal catalyst of step C) is a palladium catalyst.

152. The method of claim 151, wherein the transition metal catalyst is a palladium(II) catalyst.

153. The method of claim 151, wherein the transition metal catalyst is bis(triphenylphosphine) palladium(II) dichloride.

154. The method of claim 115, wherein the reaction of step C) comprises adding from about 0.001 equivalents to about 1.0 equivalent of the transition metal catalyst relative to the compound of formula XI.

155. The method of claim 154, wherein the reaction of step C) comprises adding from about 0.01 equivalent to about 0.5 equivalents of the transition metal catalyst.

156. The method of claim 154, wherein the reaction of step C) comprises adding about 0.02 equivalents of the transition metal catalyst.

157. The method of claim 115, wherein the transition metal catalyst of step C) further comprises a second metal salt.

158. The method of claim 157, wherein the second metal salt is a copper salt.

159. The method of claim 157, wherein the second metal salt is a copper(I) salt.

160. The method of claim 157, wherein the second metal salt is copper(I) iodide.

161. The method of claim 115, wherein the reaction of step C) comprises adding from about 0.001 equivalents to about 2.0 equivalents of the second metal salt relative to the compound of formula XI.

162. The method of claim 161, wherein the reaction of step C) comprises adding from about 0.01 equivalent to about 0.5 equivalents of the second metal salt.

163. The method of claim 161, wherein the reaction of step C) comprises adding about 0.1 equivalents of the second metal salt.

164. The method of claim 115, wherein the base of step C) is a nitrogenous base.

165. The method of claim 164, wherein the base is a trialkylamine_{(C≦18)}.

166. The method of claim 164, wherein the base is diisopropylamine.

167. The method of claim 115, wherein the reaction of step C) comprises adding from about 1.0 equivalent to about 10.0 equivalents of the base relative to the compound of formula XI.

168. The method of claim 167, wherein the reaction of step C) comprises adding from about 2.0 equivalents to about 5.0 equivalents of the base.

169. The method of claim 167, wherein the reaction of step C) comprises adding about 3.0 equivalents of the base.

170. The method of claim 115, wherein the reaction of step C) comprises adding from about 1.0 equivalent to about 10.0 equivalents of the compound of formula XII relative to the compound of formula XI.

171. The method of claim 170, wherein the reaction of step C) comprises adding from about 2.0 equivalents to about 6.0 equivalents of the compound of formula XII.

172. The method of claim 170, wherein the reaction of step C) comprises adding about 4.0 equivalents of the compound of formula XII.

173. The method of claim 115, wherein the solvent of step C) is an ether_{(C≦8)} or substituted ether_{(C≦8)}.

174. The method of claim 115, wherein the solvent of step C) is tetrahydrofuran.

175. The method of claim 115, wherein the reaction of step C) comprises performing the reaction at a temperature from about 0° C. to about 50° C.

176. The method of claim 175, wherein the temperature is from about 15° C. to about 30° C.

177. The method of claim 175, wherein the temperature is about 23° C.

178. The method of claim 175, wherein the temperature is room temperature.

179. The method of claim 115, wherein the reaction of step C) comprises performing the reaction for a time period from about 15 minutes to about 4 hours.

180. The method of claim 179, wherein the time period is about 30 minutes to about 2 hours.

181. The method of claim 179, wherein the time period is about 1 hour.

182. The method of claim 115, wherein the reaction further comprises mixing the compound of formula XI, the compound of formula XII, the base, the transition metal catalyst, and the second metal salt in the solvent.

183. The method of claim 115, wherein the oxidizing agent of step D) is a chromic compound.

184. The method of claim 183, wherein the oxidizing agent is pyridinium dichromate.

185. The method of claim 115, wherein the reaction of step D) comprises adding from about 1.0 equivalent to about 10.0 equivalents of the oxidizing agent relative to the compound of formula X.

186. The method of claim 185, wherein the reaction of step D) comprises adding from about 2.0 equivalents to about 8.0 equivalents of the oxidizing agent.

187. The method of claim 185, wherein the reaction of step D) comprises adding about 5.0 equivalents of the oxidizing agent.

188. The method of claim 115, wherein the solvent of step D) is a substituted alkane_{(C≦8)}.

189. The method of claim 115, wherein the solvent of step D) is dichloromethane.

190. The method of claim 115, wherein the reaction of step D) comprises performing the reaction at a temperature from about 0° C. to about 50° C.

191. The method of claim 190, wherein the temperature is from about 15° C. to about 30° C.

192. The method of claim 190, wherein the temperature is about 23° C.

193. The method of claim 190, wherein the temperature is room temperature.

194. The method of claim 115, wherein the reaction of step D) comprises performing the reaction for a time period from about 1 hour to about 10 hours.

195. The method of claim 194, wherein the time period is about 2 hours to about 8 hours.

196. The method of claim 194, wherein the time period is about 5 hour.

197. The method of claim 115, wherein the reaction of step D) further comprises adding 4.0 Å molecular sieves.

198. The method of claim 115, wherein the reaction of step D) further comprises mixing the compound of formula XIII, the oxidizing agent, and the molecular sieves in the solvent.

199. The method according to any one of claims 1, 114, and 115, wherein one or more steps of the reaction further comprises a deprotection step to remove one or more protecting groups.

200. The method of claim 199, wherein one or more steps of the reaction further comprises a purification step.

201. The method of claim 200, wherein the purification step comprises purifying the reaction such that the desired compound comprises greater than 90% of the total mass.

202. The method of claim 201, wherein the purification step comprises purifying the reaction such that the compound comprises greater than 95% of the total mass.

203. The method of claim 200, wherein the purification step comprises purifying the reaction via extraction or chromatography.

204. The method of claim 203, wherein the chromatography is column chromatography.

205. The method of claim 204, wherein the column chromatography is silica gel or alumina column chromatography.

206. A composition for use in treating a disease or disorder comprising modulating the activity of a G-coupled protein receptor wherein the composition comprises a compound of the formula: 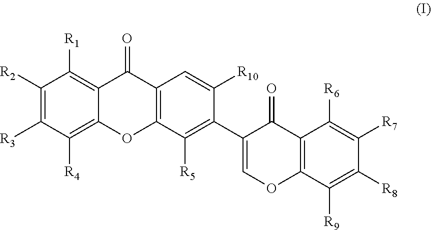
wherein:
R₁, R₂, R₃, R₄, R₆, R₇, R₈, and R₉ are each independently hydrogen, amino, carboxy, halo, hydroxy, mercapto, or
alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅,
wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and
R₅ and R₁₀ are each independently hydrogen, acyl_{(C≦12)}, or substituted acyl_{(C≦12)}; or
a compound of the formula: 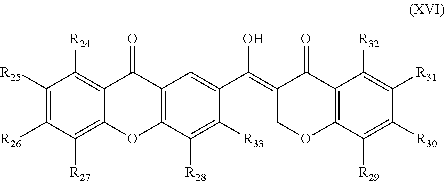
wherein:
R₂₄, R₂₅, R₂₆, R₂₇, R₂₉, R₃₀, R₃₁, and R₃₂ are each independently hydrogen, amino, carboxy, halo, hydroxy, mercapto, or
alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅,
wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group;
R₂₈ is hydrogen, acyl_{(C≦12)}, or substituted acyl_{(C≦12)}; and
R₃₃ is hydrogen, alkyl_{(C≦12)}, substituted alkyl_{(C≦12)}, acyl_{(C≦12)}, or substituted acyl_{(C≦12)};
or a pharmaceutically acceptable salt or tautomer thereof.

207. The composition of claim 206, wherein the G-coupled protein receptor is a succinate receptor.

208. The composition of claim 207, wherein the succinate receptor is G-coupled protein receptor succinate receptor 1.

209. The composition of claim 206, wherein the disease or disorder is excessive angiogenesis of the retina or cornea.

210. The composition of claim 206, wherein the disease or disorder is retinopathy.

211. The composition of claim 210, wherein the retinopathy is caused by excessive angiogenesis of the retina and cornea.

212. The composition of claim 206, wherein the disease or disorder is an infection.

213. The composition of claim 212, wherein treating the infection comprises activating a dendritic cell.

214. The composition of claim 206, wherein the disease or disorder is cancer.

215. The composition of claim 214, wherein the cancer is a carcinoma, sarcoma, lymphoma, leukemia, melanoma, mesothelioma, multiple myeloma, or seminoma.

216. The composition of claim 214, wherein the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

217. The composition of claim 206, wherein the compound is administered orally, intravenously, topically, intraocularly, or locally.

218. The composition of claim 206, wherein the composition further comprises a second therapeutic agent.

219. The composition of claim 218, wherein the second therapeutic agent is succinic acid or a salt thereof, a chemotherapeutic, surgery, an immunotherapy, a genetic therapy, an antibiotic, or an anti-viral agent.

220. A composition for use in treating a disease or disorder associate with inflammation or vascular proliferation comprising a compound of the formula: 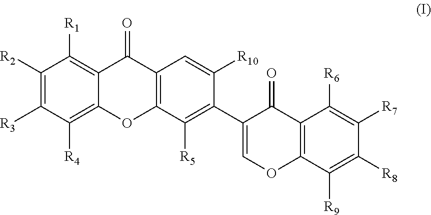
wherein:
R₁, R₂, R₃, R₄, R₆, R₇, R₈, and R₉ are each independently hydrogen, amino, carboxy, halo, hydroxy, mercapto, or
alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅,
wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and
R₅ and R₁₀ are each independently hydrogen, acyl_{(C≦12)}, or substituted acyl_{(C≦12)}; or
a compound of the formula: 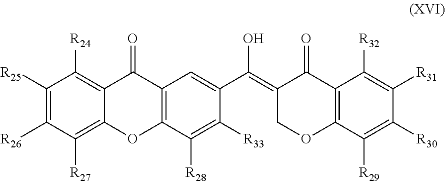
wherein:
R₂₄, R₂₅, R₂₆, R₂₇, R₂₉, R₃₀, R₃₁, and R₃₂ are each independently hydrogen, amino, carboxy, halo, hydroxy, mercapto, or
alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅,
wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group;
R₂₈ is hydrogen, acyl_{(C≦12)}, or substituted acyl_{(C≦12)}; and
R₃₃ is hydrogen, alkyl_{(C≦12)}, substituted alkyl_{(C≦12)}, acyl_{(C≦12)}, or substituted acyl_{(C≦12)};
or a pharmaceutically acceptable salt or tautomer thereof.

221. The composition of claim 220, wherein the disease or disorder is a cardiovascular disease or disorder, a dermatological disease or disorder, a metabolic disease or disorder, cancer, a gastrointestinal or liver disease or disorder, a hematological disease or disorder, a reproductive disease or disorder, an endocrinal disease or disorder, an inflammatory disease or disorder, a muscle-skeleton disease or disorder, a neurological disease or disorder, a urological disease or disorder, a respiratory disease or disorder, and an ophthalmological disease or disorder.

222. The composition of claim 220, wherein the disease or disorder is cancer, diabetic retinopathy, or an infection.

223. The composition of claim 220, wherein the disease or disorder is associated with dysregulation of a G-coupled protein receptor.

224. The composition of claim 223, wherein the G-coupled protein receptor is a succinate receptor.

225. The composition of claim 223, wherein the G-coupled protein receptor is G-coupled protein receptor succinate receptor 1.

226. The composition of claim 223, wherein the compound acts as an agonist of G-coupled protein receptor succinate receptor 1.

227. The composition of claim 223, wherein the compound acts as an antagonist of G-coupled protein receptor succinate receptor 1.

228. The composition of claim 220, wherein the composition further comprises a second therapeutic agent.

229. The composition of claim 228, wherein the second therapeutic agent is succinic acid or a salt thereof, a chemotherapeutic, surgery, an immunotherapy, a genetic therapy, an antibiotic, or an anti-viral agent.

230. A composition for use in promoting nerve regeneration comprising succinic acid or a salt thereof and a compound of the formula: 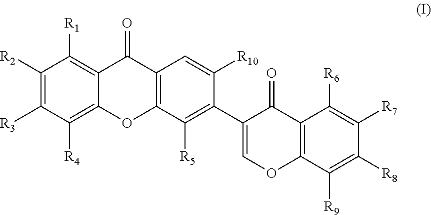
wherein:
R₁, R₂, R₃, R₄, R₆, R₇, R₈, and R₉ are each independently hydrogen, amino, carboxy, halo, hydroxy, mercapto, or
alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅,
wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group; and
R₅ and R₁₀ are each independently hydrogen, acyl_{(C≦12)}, or substituted acyl_{(C≦12)}; or
a compound of the formula: 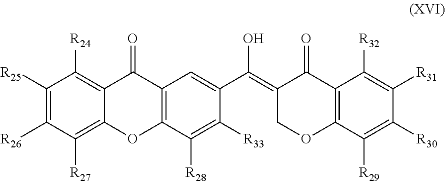
wherein:
R₂₄, R₂₅, R₂₆, R₂₇, R₂₉, R₃₀, R₃₁, and R₃₂ are each independently hydrogen, amino, carboxy, halo, hydroxy, mercapto, or
alkyl_{(C≦12)}, aryl_{(C≦12)}, acyl_{(C≦12)}, alkoxy_{(C≦12)}, or a substituted version of any of these groups, or —OX₁, —NX₂X₃, —SX₄, or —C(O)OX₅,
wherein: X₁ is a hydroxy protecting group, X₂ and X₃ are each independently hydrogen or a monovalent amino protecting group, X₂ and X₃ are taken together and are a divalent protecting group, X₄ is a thiol protecting group, and X₅ is a carboxy protecting group;
R₂₈ is hydrogen, acyl_{(C≦12)}, or substituted acyl_{(C≦12)}; and
R₃₃ is hydrogen, alkyl_{(C≦12)}, substituted alkyl_{(C≦12)}, acyl_{(C≦12)}, or substituted acyl_{(C≦12)};
or a pharmaceutically acceptable salt or tautomer thereof.

231. The composition of claim 230, wherein the composition comprised contacting a nerve of the central nervous system, the peripheral nervous system or both with the compound.

232. The composition of claim 230, wherein the succinate salt is sodium succinate.

233. The composition of claim 230, wherein the composition leads to axonal regeneration.

234. The composition of claim 230, wherein the composition leads to axonal myelination.

235. The composition of claim 230, wherein the composition promotes angiogenesis.

236. The composition of claim 230, wherein the composition promotes cellular survival.

237. The composition of claim 230, wherein the composition comprises modulating the activity of G-coupled protein receptor succinate receptor 1.

238. The composition of claim 230, wherein promoting neural regeneration modulates the effects of a disease or disorder.

239. The composition of claim 238, wherein the neural regeneration mitigates the effects of a spinal cord injury.

240. The composition of claim 238, wherein the neural regeneration mitigates the effects of a disease or disorder.

241. The composition of claim 240, wherein the disease or disorder is a neurological disease or disorder.

242. The composition of claim 241, wherein the neurological disease or disorder is Alzheimer's disease or Parkinson's disease.

243. The composition of claim 230, wherein the composition further comprises a second therapeutic agent.

244. A compound of the formula: 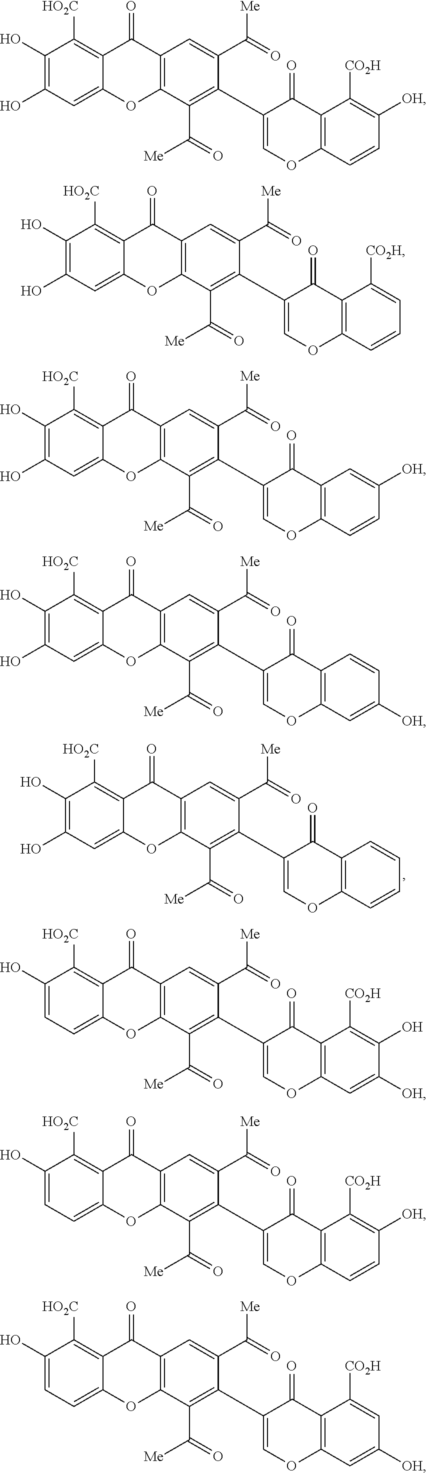 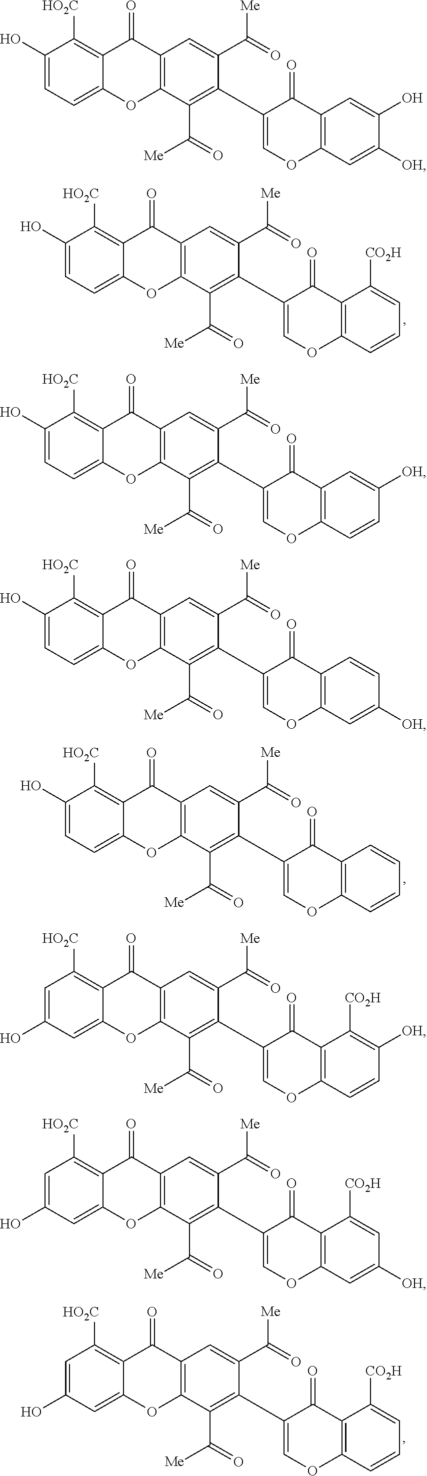 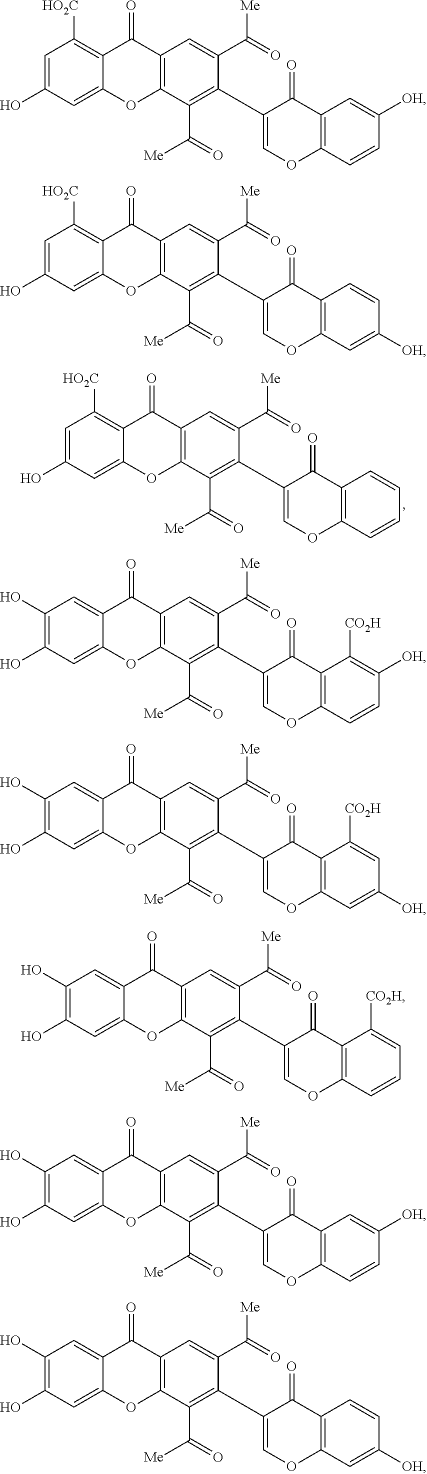 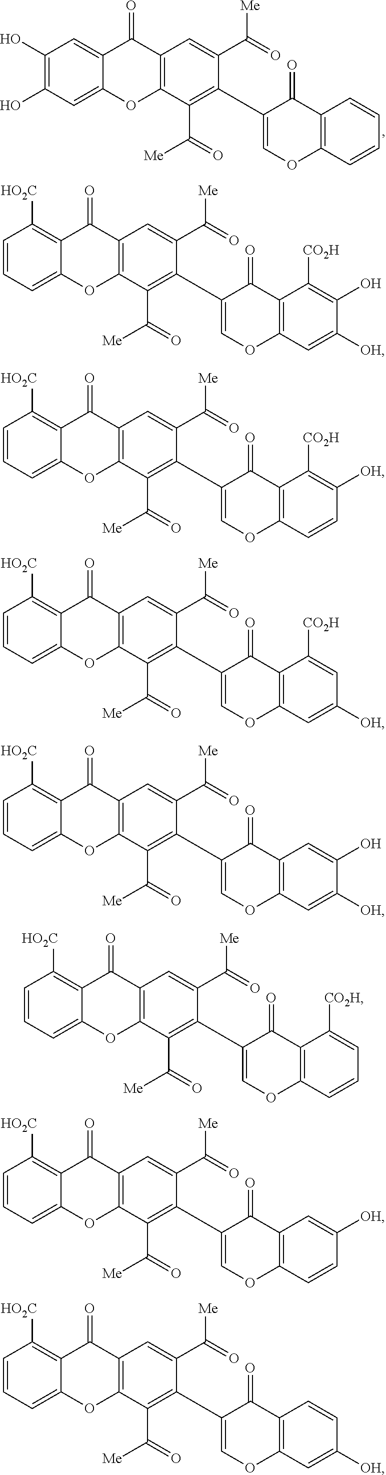 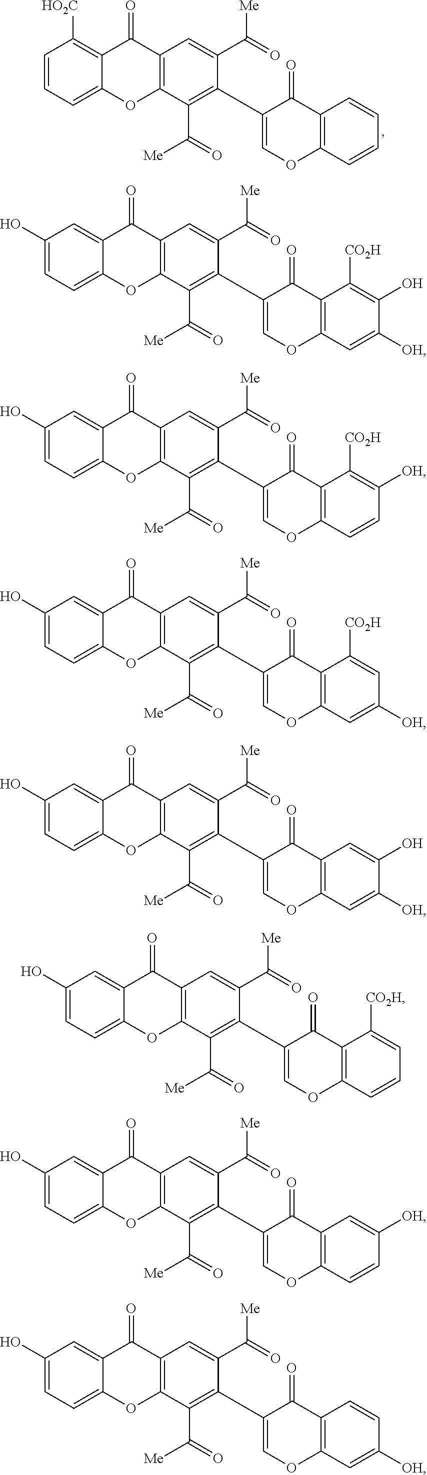 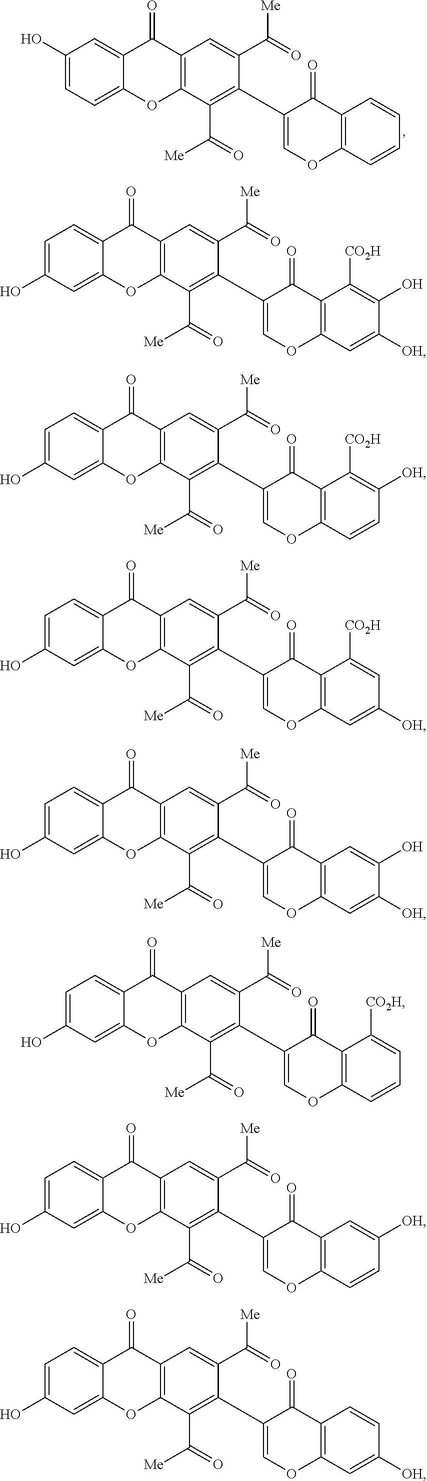 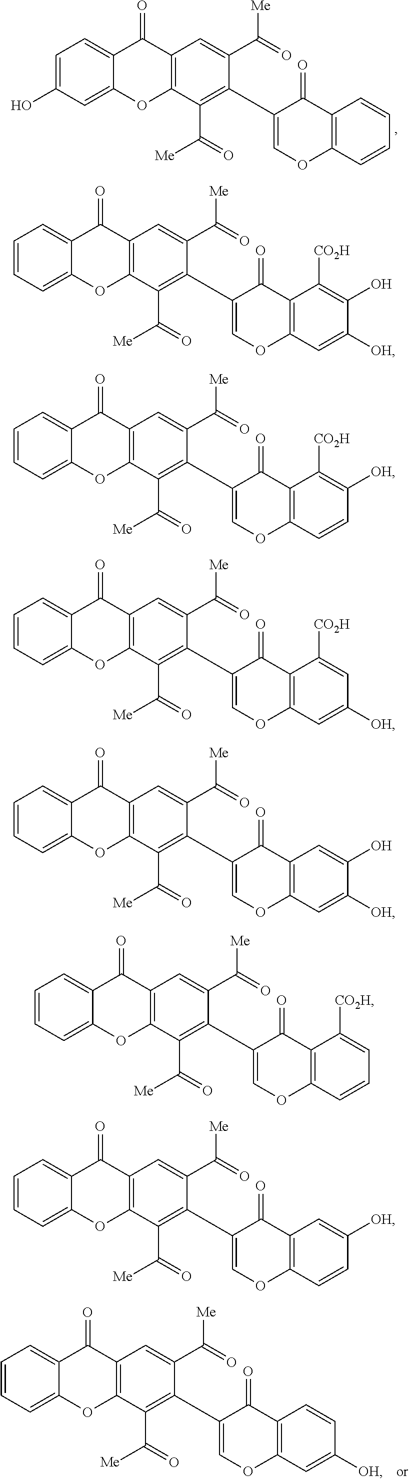 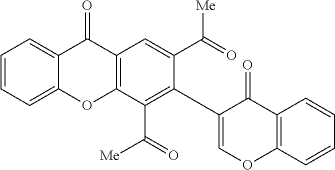 [SMILES: CC(=O)c1cc2c(=O)c3ccccc3oc2c(C(C)=O)c1-c1coc2ccccc2c1=O]
or a pharmaceutically acceptable salt or tautomer thereof.

245. A pharmaceutical composition comprising a compound of claim 244 and a pharmaceutically acceptable excipient.

246. The composition of claim 245, wherein the composition is formulated for administration locally, orally, systemically, intravenously, topically, or intraocularly.

247. The composition of claim 245, wherein the composition is formulated in a fixed dose form
